(19) United States
(12) Patent Application Publication
(10) Pub. No.: US 2024/0207312 A1
(43) Pub. Date: Jun. 27, 2024

(54) CHIMERIC ANTIGEN RECEPTOR (CAR)-T CELLS

(71) Applicant: Imperial College Innovations Limited, London (GB)

(72) Inventors: Xiaoning Xu, London (GB); Weiwei Ma, London (GB); Yuxin Qian, London (GB)

(73) Assignee: Imperial College Innovations Limited, London (GB)

(21) Appl. No.: 18/287,542

(22) PCT Filed: Apr. 21, 2022
(86) PCT No.: PCT/GB2022/051002

§ 371 Date: Oct. 19, 2023

(30) Foreign Application Priority Data
Apr. 21, 2021 (GB) 2105682.5

(51) Int. Cl. A61K 35/17; A61K 39/00; A61P 35/02; C07K 16/24; C07K 16/28; C12N 5/0783; C12N 15/86

(52) U.S. Cl. CPC A61K 35/17; A61K 39/4611; A61K 39/4631; A61K 39/464411; A61K 39/46444; A61P 35/02; C07K 16/246; C07K 16/2833; C12N 5/0636; C12N 15/86; A61K 2239/13; A61K 2239/48

(57) ABSTRACT

The present invention relates to chimeric antigen receptor (CAR)-T cells, and particularly, although not exclusively, to their use in immunotherapy, and for treating, preventing or ameliorating cancer, such as T-cell lymphomas, various microbial infections, such as HIV and TB, and also autoimmune disease. The invention is especially concerned with the use of CAR-engineered mucosal-associated invariant T (MAIT) cells, and to novel methods for stimulating, isolating, and expanding highly purified MAIT cells, which can then be engineered into such CAR-MAIT cells. The invention is also concerned with methods for expansion of MAIT cells in vitro.

The present invention relates to chimeric antigen receptor (CAR)-T cells, and particularly, although not exclusively, to their use in immunotherapy, and for treating, preventing or ameliorating cancer, such as T-cell lymphomas, various microbial infections, such as HIV and TB, and also autoimmune disease. The invention is especially concerned with the use of CAR-engineered mucosal-associated invariant T (MAIT) cells, and to novel methods for stimulating, isolating, and expanding highly purified MAIT cells, which can then be engineered into such CAR-MAIT cells. The invention is also concerned with methods for expansion of MAIT cells in vitro. The invention extends to genetic constructs per se, and to their use in generating the CAR-MAIT cells, and to transduced CAR-MAIT cells per se. The invention also extends to various medical uses of the constructs and transduced CAR-MAIT cells, and to pharmaceutical compositions comprising these constructs and CAR-MAIT cells.

T-cell lymphoma are a heterogeneous group of clinically aggressive diseases, including peripheral T-cell lymphoma (PTCL), such as adult T-cell leukaemia/lymphoma (ATL) caused by human T-lymphotropic virus type I (HTLV-1), and cutaneous T-cell lymphoma (CTCL), such as Sezary Syndrome (SS). T-cell malignancies are more difficult to treat than B-cell malignancies. ATL and SS represent a rare and often aggressive type of T-cell lymphoma and there have not been enough patients enrolled 20 in randomized trials to establish treatment standards. As a result, common first-line therapies used are the same as those used to treat other types of T-cell lymphomas. For example, currently licensed drugs for the treatment of T cell malignancies include chemotherapeutic agents, biological response modifiers (e.g. interferon, bexarotene and HDAC inhibitors), monoclonal antibodies (alemtuzumab, mogamulizumab, 25 brentuximab), haematopoietic stem cell transplantation (HSCT), and extra corporeal photopheresis (ECP). However, no single treatment regimen is known to be superior to others in its overall response rate or duration of response. Although allogeneic (HSCT) has been the only potential curative regimen, a significant number of patients may not be fit for HSCT because of advanced age and comorbidities in addition to HSCT-associated mortality. There is, therefore, a need for more effective treatment, as the median survival with current treatment for ATL is only 8 months (Katsuya et al., 2015).

The recent FDA approved chimeric antigen receptor (CAR)-based T-cell therapy (CAR-T) has been regarded as one of the most significant breakthroughs in the treatment of B-cell malignancies by targeting CD19 antigen achieving nearly 100% remission (Park et al., 2018). Despite the effectiveness of CAR-T treatment for B-cell malignancies, however, a similar approach for targeting T cell-derived malignancies has not been well-established. Like most B-cell malignancies containing identical rearrangements of the immunoglobulin gene (i.e. clonal) and expressing pan-B-cell markers, such as CD19, the majority (>95%) of ATL and CTCL is derived from a dominant T cell clone expressing a defined T-cell receptor (TCR) gene (i.e. a clonal TCR-Vbeta chain) and the pan-T helper cell marker CD4. Therefore, targeting pan-T cell markers, such as CD4 or the TCR-Vb chain, by monoclonal antibodies (mAb) for the treatment of T lymphoma have been tested but have resulted in only a partial regression in small clinical trials.

Current CAR-T therapy is mainly based on conventional αβ T cells. However, the antigen recognition of αβ T cells is severely limited by MHC, which make it suitable for autologous therapy, but very difficult for allogeneic adoptive transfer. Moreover, due to the inadequate tumour infiltration of αβ T cells, conventional CAR-T therapy shows low efficacy in solid tumours, such as CTCL, but promising potential in liquid tumour therapy. However, conventional CAR-T cell therapy has some major drawbacks limiting its further application. For example, current CAR-T therapy is: (i) limited by autologous transfusion due to graft-vs-host diseases (GVHD), (ii) limited by on-target/off-tumour toxicity with cytokine release syndrome; (iii) disadvantages of autologous CAR-T, such as variability of patient's T cell function, product standardization and cost.

There is, therefore, a need to provide improved immunotherapies for T-cell malignancies, such as T-cell lymphoma, including PTCL and CTCL, and also for treating microbial infections, such as HIV and TB.

In order to treat T-cell malignancies, the inventors focused their attention on mucosal associated invariant T cells (MAIT cells), which are a subset of T cells in the immune system that display innate, effector-like qualities. MAIT cells are defined by an invariant usage of the T-cell receptor chain Vα7.2, restricted by the major histocompatibility complex (MHC)-Ib-related protein, MR1, and exhibit high 30 expression of the C-type lectin CD161 and IL18 receptor. In humans, MAIT cells are found in the blood, liver, lungs, and mucosa, defending against microbial activity and infection. The MHC class I-like protein, MR1, is responsible for presenting bacterially-produced vitamin B metabolites, such as 5-OP-RU, to MAIT cells. After the presentation of foreign antigen by MR1, MAIT cells secrete pro-inflammatory cytokines and are capable of lysing bacterially-infected cells.

Current MAIT cell expansion methods require the use of human allogenic feeder cells to support the growth of MAIT cells in vitro culture, which is difficult for a large-scale production and quality controls for adaptive immunotherapy in humans. In addition, the MAIT cells produced using these known methods contain a percentage of other cell subsets, such as conventional CD4+ T cells and CD8+ T cells, which therefore renders the resultant MAIT isolate wholly unsuitable for its use in allogeneic adoptive transfer, because those cell subsets will cause Graft verses host disease (GvHD).

As such, there is also a need for an improved method for stimulating and isolating pure MAIT cell cultures, without the need of allogeneic feeder cells, to scale up MAIT cell production therefore be used in allogeneic adoptive transfer.

As described in the Examples, the inventors have developed a novel method for stimulating and isolating highly pure cultures of MAIT cells from the human PBMCs. The inventors have also developed several novel genetic constructs and vectors (referred to herein as “CART4” and “CARTVb7.1”) each of which encode a chimeric antigen receptor (CAR), and which are then transduced into the pure MAIT cells, thereby producing CAR-MAIT cells which specifically target either the CD4 molecule (using “CART4”) or TCR-Vbeta 7.1 chain (using “CARTVb7.1”) on T-cells. This was achieved by creating the novel genetic constructs comprising the scFv of either (i) an anti-CD4 mAb (e.g. Hu5A8) or (ii) an anti-TCR-Vb 7.1 mAb (e.g. 3G5), with CD28/4-1BB/CD3zeta chain signalling moieties to form a third generation CAR. These CARs were then transduced into MAIT cells purified from peripheral blood mononuclear cells (PBMCs) to create resultant CAR-MAIT cells targeting either CD4 on a T-cell or the TCR-Vbeta 7.1 chain on a T-cell. The inventors have demonstrated that these CAR-MAIT cells surprisingly exhibited anti-T lymphoma activity comparable, and even superior, with conventional CAR-T cells.

Thus, in a first aspect of the invention, there is provided a mucosal-associated invariant T (MAIT) cell expressing a chimeric antigen receptor (CAR).

As discussed herein, MAIT cells are non-conventional and innate-like T cells expressing an invariant T-cell receptor (TCR), which are highly conserved during mammalian evolution, recognize microbial antigens presented by the MR1 protein, and are present in human blood and maintain tissue homeostasis for broad antimicrobial responsiveness. In addition, advantageously, the antigen recognition mechanism of MAIT cells is MHC-independent, which makes the MAIT cell an exciting candidate for allogeneic T cell killing therapies, such that it is not limited to autologous therapy, as in current T cell therapies, which are MHC-dependent. In other words, MAIT cells have low allogenic reactivity, and are less prone to inducing graft vs host disease (GVHD) in humans, and so represent an ideal T cell subset for allogenic CAR-T therapy. Comparatively, the CART-MAIT cells of the invention and resultant cellular therapy can be easily allogeneic transferred, and the endogenous characters of the MAIT cell make it a promising candidate to infiltrate into peripheral tissues for solid tumour treatments. Accordingly, the CAR-MAIT cells are able to effectively infiltrate into the solid tumours, which makes MAIT cell-based cellular therapy a promising therapy for solid tumours as well as liquid tumours.

Advantageously, the CAR-MAIT cells of the invention may be used to treat T-cell malignancies, such as adult T-cell leukaemia/lymphoma (ATL) caused by human T-lymphotropic virus type I (HTLV-1) and cutaneous T-cell lymphomas (CTCL), such as Sezary Syndrome. It will be appreciated that T-cell malignancies are a heterogeneous group of clinically aggressive diseases, and are significantly more difficult to treat than B-cell malignancies. Advantageously, the CAR-MAIT therapy is expected to show increased efficacy against solid tumours and be allogenic, thereby negating the requirement for autologous transfer and so positioning it as an off-the-shelf therapy.

Preferably, in one embodiment, the CAR-MAIT cell expresses a CAR which targets a CD4 antigen on a T-cell. Thus, preferably the CAR is specific for a CD4 antigen on a T-cell. It will be appreciated that the CD4 antigen is a glycoprotein found on the surface of immune cells, such as T helper cells, monocytes, macrophages and dendritic cells (T-cell surface glycoprotein CD4 [Homo sapiens] UniProt No. PO1730.1; NCBI reference sequence NP_000607.1). One embodiment of the polypeptide sequence of the CD4 antigen is represented herein as SEQ ID No: 1, as follows:

MNRGVPFRHL LLVLQLALLP AATQGKKVVL GKKGDTVELT
CTASQKKSIQ FHWKNSNQIK ILGNQGSFLT
KGPSKLNDRA DSRRSLWDQG NFPLIIKNLK IEDSDTYICE
VEDQKEEVQL LVFGLTANSD THLLQGQSLT
LTLESPPGSS PSVQCRSPRG KNIQGGKTLS VSQLELQDSG
TWTCTVLQNQ KKVEFKIDIV VLAFQKASSI
VYKKEGEQVE FSFPLAFTVE KLTGSGELWW QAERASSSKS
WITFDLKNKE VSVKRVTQDP KLQMGKKLPL
HLTLPQALPQ YAGSGNLTLA LEAKTGKLHQ EVNLVVMRAT
QLQKNLTCEV WGPTSPKLML SLKLENKEAK
VSKREKAVWV LNPEAGMWQC LLSDSGQVLL ESNIKVLPTW
STPVQPMALI VLGGVAGLLL FIGLGIFFCV
RCRHRRRQAE RMSQIKRLLS EKKTCQCPHR FQKTCSPI

Therefore, preferably the CAR is specific for a CD4 antigen which comprises an amino acid sequence substantially as set out in SEQ ID No:1, or a variant or fragment thereof.

Preferably, in another embodiment, the CAR-MAIT cell expresses a CAR which targets a T-cell receptor (TCR) beta-chain variable region (Vbeta) on a T-cell. It will be appreciated that the T-cell receptor (TCR) is a protein complex found on the surface of T cells, or T lymphocytes, that is responsible for recognizing fragments of antigen as peptides bound to major histocompatibility complex (MHC) molecules. The TCR is composed of two different protein chains. In humans, in 95% of T cells the TCR consists of an alpha (α) chain encoded by TRA, and a beta (β) chain encoded by TRB.

Table 1 below lists Vbeta regions on T-cells, with the associated encoding gene, and any one or more of these may be targeted by the CAR on the CAR-MAIT cells of the invention.

TABLE 1
Beta-chain variable regions (Vebta) on T-cells

| Vbeta | Gene |
|---|---|
| Vb₁ | TRBV₉ |
| Vb₂ | TRBV₂₀₋₁ |
| Vb₃ | TRBV₂₈ |
| Vb₄ | TRBV₂₉₋₁ |
| Vb_{5.1} | TRBV₅₋₁ |
| Vb_{5.2} | TRBV₅₋₆ |
| Vb_{5.3} | TRBV₅₋₅ |
| Vb_{7.1} | TRBV₄₋₁, TRBV₄₋₂, TRBV₄₋₃ |
| Vb_{7.2} | TRBV₄₋₃ |
| Vb₈ | TRBV₁₂₋₃, TRBV₁₂₋₄ |
| Vb₉ | TRBV₃₋₁ |
| Vb₁₁ | TRBV₂₅₋₁ |
| Vb₁₂ | TRBV₁₀₋₃ |
| Vb_{13.1} | TRBV₆₋₅, TRBV₆₋₆, TRBV₆₋₉ |
| Vb_{13.2} | TRBV₆₋₂ |
| Vb_{13.6} | TRBV₆₋₆ |
| Vb₁₄ | TRBV₂₇ |
| Vb₁₆ | TRBV₁₄ |
| Vb₁₇ | TRBV₁₉ |
| Vb₁₈ | TRBV₁₈ |
| Vb₂₀ | TRBV₃₀ |
| Vb_{21.3} | TRBV₁₁₋₂ |
| Vb₂₂ | TRBV₂ |
| Vb₂₃ | TRBV₁₃ |

The CAR-MAIT cell may express a CAR which targets a plurality of T-cell receptor (TCR) beta-chain variable regions (Vbeta) on a T-cell. Preferably, the plurality of Vbeta regions may be selected from a group of Vbeta regions shown in Table 1. For example, the CAR-MAIT cell may express a CAR which targets at least two, or at least three or at least four T-cell receptor (TCR) beta-chain variable regions (Vbeta) on a T-cell, preferably as listed in Table 1. The CAR-MAIT cell may express a CAR which targets at least five, or at least six or at least seven T-cell receptor (TCR) beta-chain variable regions (Vbeta) on a T-cell, preferably as listed in Table 1. The plurality of TCR V beta regions may be the same or different V beta regions.

The following Vb families are believed to be frequently associated with T-cell lymphoma, i.e. Vb 1, Vb 2, Vb 3, Vb 5.1, Vb 7.1, Vb 8, Vb 12, Vb 13.1, Vb 17, and Vb 20. Therefore, in a preferred embodiment, the CAR-MAIT cell expresses a CAR which targets one or more TCR Vbeta region on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20. Preferably, the CAR-MAIT cell expresses a CAR which targets at least two or three TCR Vbeta regions on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20.

Thus, preferably the CAR is specific for at least one or more TCR Vbeta region on a T-cell, and most preferably the TCR-Vbeta 7.1 chain. One embodiment of the polypeptide sequence of TCR Vbeta 7.1 region (H. sapiens rearranged TCR Vbeta 7.1 UniProtKB/Swiss-Prot: AoA577.1) is represented herein as SEQ ID No: 2, as follows:
MGCRLLCCAVLCLLGAVPIDTEVTQTPKHLVMGMTNKKSLKCEQHMGHRAMYWYKOKAKKPPELMFVYSYEKLSINES VPSRESPECPNSSLLNLHLHALQPEDSALYLCASSQ

Therefore, preferably the CAR is specific for at least one or more TCR Vbeta region (and more preferably the TCR-Vbeta 7.1 chain) which comprises an amino acid sequence substantially as set out in SEQ ID No:2, or a variant or fragment thereof.

Preferably, the CAR-MAIT cell is configured to kill target T cells directly by inducing apoptosis.

Preferably, the CAR-MAIT cell comprises one or more coding sequence, which allows for the CAR-MAIT cells to be controllably or inducibly eliminated, for example in the case of an adverse patient reaction. The one or more coding sequence may be known to the skilled person as a so-called “suicide gene”.

In one embodiment, therefore, the one or more coding sequence may encode epidermal growth factor receptor (EGFR), or truncated epidermal growth factor receptor (tEGFR) (refs) (UniProt No. P00533; NCBI reference sequence NP_001333826.1). The expression of tEGFR can be controlled by anti-EGFR mAb (Cetuximab) for monitoring or depletion of the CAR-T cells in a patient. It will be appreciated that EGFR is known as HER1 in humans, and is a transmembrane protein that is a receptor for members of the epidermal growth factor (EGF) of extracellular protein ligands (UniProt No. P01133; NCBI reference sequence NP_001171601.1).

In another embodiment, the one or more coding sequence may encode inducible caspase-9 (iC9) (Mol. Therapy, Diaconu et al., 580, 25, 3, March 2017). iC9 is a modified human Caspase-9 (UniProt No. P55211; NCBI reference sequence NP_001220.2) fused to the human FK506 binding protein (UniProt No. P62942; NCBI reference sequence NP_000792.1) to allow conditional dimerization using a chemical inducer of dimerization (caspase inducible drug (CID), Rimiducid), which triggers apoptosis of the CAR-T cells expressing the fusion protein.

Preferably, the CAR-MAIT cell comprises a coding sequence encoding truncated epidermal growth factor receptor (tEGFR) and/or inducible caspase-9 (iC9). More preferably, the CAR-MAIT cell comprises a coding sequence encoding truncated epidermal growth factor receptor (tEGFR) and inducible caspase-9 (iC9).

It will be appreciated that the CAR-MAIT cell is produced by transducing a MAIT cell with a nucleic acid or genetic construct encoding the CAR. It is important for a highly purified culture of MAIT cells are used in the CAR transduction step to produce T-cell specific and active CAR-MAIT cells. Hence, the inventors have developed an effective method for isolating purified MAIT cells from human peripheral blood monocyte cells (PBMCs) by combining magnetic activation cell sorting (MACS) and fluorescence activated cell sorting (FACS) methods, such that resultant method yields a large amount of MAIT cells with a high expansion fold.

Preferably, therefore, the MAIT cell is isolated from human peripheral blood monocyte cells (PBMCs). Preferably, the MAIT cells is isolated from PBMCs by magnetic activated cell sorting (MACS) and/or fluorescence activated cell sorting (FACS), more preferably both MACS and FACS. The inventors believe that their MAIT isolation method is novel per se.

Thus, in a second aspect, there is provided a method of isolating a MAIT cell, the method comprising:
(i) providing peripheral blood monocyte cells (PBMCs); and
(ii) subjecting the PBMCs to magnetic activated cell sorting (MACS) and/or fluorescence activated cell sorting (FACS) to isolate MAIT cells therefrom.

Preferably, the method of the invention results in the isolation of pure ex vivo MAIT cells. In one embodiment, the method comprises subjecting the PBMCs to either MACS or FACS to isolate MAIT cells therefrom. However, preferably the method comprises subjecting the PBMCs to both MACS and FACS to isolate the MAIT cells therefrom. Preferably, the PBMCs are subjected to MACS first followed by FACS. Preferably, the method comprises isolating TCR Vα7.2+ cells from the PBMCs by MACS, and then subjecting those cells to FACS using by MR1-5-OP-RU tetramer staining, to isolate the MAIT cells.

The MACS procedure (in step (ii)) may comprise collecting the PBMCs, and then washing the cells with a binding buffer. The supernatant may be discarded, and a resultant cell pellet may be resuspended in a MACS buffer (e.g. at a concentration of 1×10⁷/100 μl). The solution may be contacted with a Phycoerythrin (PE) anti-human TCR Vα7.2 antibody (e.g. at a ratio of 1:100). The solution may be mixed, and it may then be incubated (e.g. for 30 min on ice). The cells may be washed with MACS buffer (e.g. by centrifuging 5 min at 300×g). The cells may be resuspended in MACS buffer (e.g. at a concentration of 10⁷/80 μl). The suspension may be contacted with anti-PE microbeads, and then it may be incubated (e.g. for 20 min on ice). The cells may be washed (e.g. 10 times volume of MACS buffer). The solution may be centrifuged (e.g. at 300×g for 5 min). The cells may be resuspended (e.g. in 1 ml MACS buffer). An MS column may be prewashed with a MACS buffer and assembled on the magnet. The cells may be applied to the column and MACS carried out. The column may then be washed one or more times (e.g. each time with MACS buffer). The column may be removed from the magnet, and bound cells may be eluted from the column (e.g. in MACS buffer).

The FACS procedure (in step (ii)) may comprise collecting magnet-separated cells and then centrifuging (e.g. at 300×g for 5 min). The cells may be resuspended (e.g. at a concentration of 10⁷/100 μl with FACS buffer). The solution may be contacted with BV421-labeled human 5-OP-RU MR1 tetramer (e.g. at a ratio of 1:500) and APC-H7-conjugated anti-human CD3 (e.g. at a ratio of 1:200). The solution may then be incubated (e.g. for 20 min on ice). The cells may be washed (e.g. with 10 times volume of FACS buffer). The solution may be centrifuged (e.g. at 300×g for 5 min). The cells may be resuspended (e.g. in 2 ml FACS buffer). A FACS sorter (e.g. the BD Prodigy Sorter) may then be loaded with a cell sample and FACS carried out.

Before the isolated MAIT cells are transduced with the nucleic acid encoding the CAR, preferably the MAIT cells are activated in a subsequent step after step (ii) in the method of the second aspect. Hence, the method further comprises activating the isolated MAIT cells with an anti-CD3 antibody, preferably in vitro. The method comprises activating the isolated MAIT cells with an anti-CD28 antibody, preferably in vitro. Preferably, the isolated MAIT cells are activated with both anti-CD3 and anti-CD28 antibodies, either substantially simultaneously or sequentially. Sequential activation may comprise contacting the MAIT cells with the anti-CD3 first followed by the anti-CD28 antibody, or with the anti-CD28 antibody first and then the anti-CD3 antibody. Contacting with the antibody may be for at least one day, two days or three days.

The MAIT cell activation procedure may comprise collecting the sorted MAIT cells by centrifuge (e.g. at 300×g for 5 min). The supernatant may be discarded and the pellet may be resuspended (e.g. in R10 medium to 10⁶ cells/ml). The solution may be contacted with Dynabeads Human T-Activator CD3/CD28 (e.g. by vortex for 30 sec).

The desired volume of Dynabeads may be transferred to a tube. An equal volume of buffer may be added to the tube and it may be mixed (e.g. by vortex for 5 sec). The tube may be placed on a magnet (e.g. for 1 min) and the supernatant may be discarded. The tube may be removed from the magnet and the washed Dynabeads may resuspended (e.g. in the R10 medium). A desired volume of Dynabeads may be contacted with the cell suspension (e.g. to obtain a bead-to-cell ratio of about 1:1 with 100 IU/ml IL-2 in 24-well-plate in 37° C. incubator).

The method may then comprise transducing the isolated and now activated MAIT cells with the nucleic acid encoding the CAR.

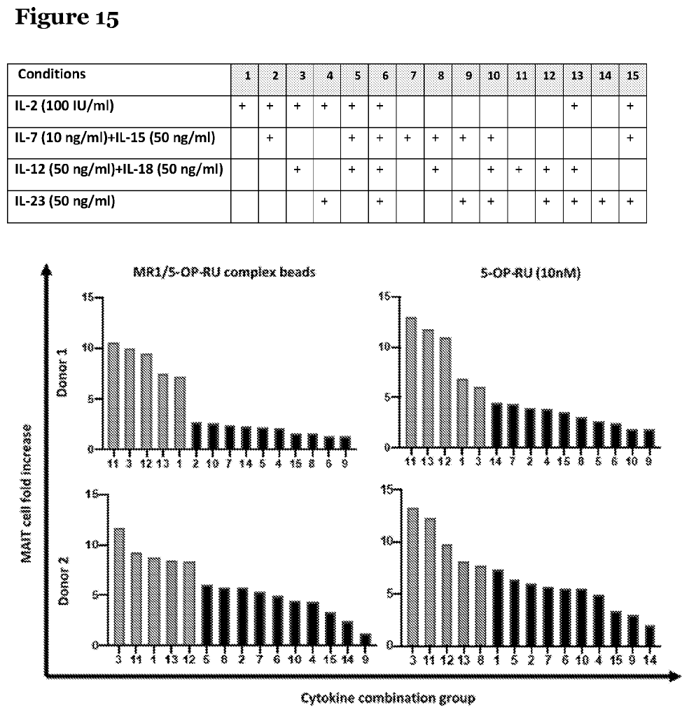

MAIT cells are a subset of innate T cells defined as CD3+TCRVα7.2+CD161+ cells which recognise the MHC class I-like molecule, MR1. Previous research has shown that MAIT cells can be expanded in vitro but requiring the presence of allogenic feeder cells. However, a problem with this method is that it is difficult for a large-scale production and quality controls. As described in Example 10 and as shown in FIG. 15, the inventors have now developed a surprisingly effective method for expansion of MAIT cells in vitro by initially stimulating the PBMCs with an antigen (5-OP-RU) loaded MR1 tetramer beads or 5-OP-RU (alone), in the presence of a combination of various cytokines for up to 6 days in vitro culture. The MAIT cells were then isolated by MACS or FACS sorting, and subsequently expanded further by anti-CD3/CD28 beads for CAR-based therapies, as discussed above.

Accordingly, in a preferred embodiment, the method may comprise stimulating the PBMCs before they are subjected to the MACS and/or FACS step (i.e. step ii). Preferably, this initial stimulating step comprises contacting the PBMCs with (a) an antigen comprising either MR1/5-OP-RU or 5-OP-RU; and/or (b) a cytokine. Preferably, the stimulating step comprises contacting the PBMCs with (a) an antigen comprising either MR1/5-OP-RU or 5-OP-RU; and (b) a cytokine. The stimulation step may comprises contacting the PBMCs with the antigen and/or cytokine for at least 1 day, 2 days or 3 days. The stimulation step may last for at least 4 days, 5 days or 6 days. Preferably, the stimulation step comprises contacting the PBMCs with the antigen and/or cytokine in an in vitro culture.

MR1/5-OP-RU and 5-OP-RU are described in WO 2015/149130 the entire contents of which are incorporated herein by reference. Accordingly, preferably the antigen comprises either MR1/5-OP-RU or 5-OP-RU, as described in WO 2015/149130 (PCT/AU2015/050148).

The cytokine may be any interleukin. However, preferably the cytokine may be one or more interleukin selected from a group consisting of IL-2, IL-7, IL-12, IL-15, IL-18 and IL-23, or any combination thereof. The concentration of the interleukin may be at least 5, 10 or 20 ng/ml, preferably at least 30, 40 or 50 ng/ml.

For example, the one or more interleukin may comprise (i) IL-2 alone (condition 1 in FIG. 15); (ii) IL-12 and IL-18 (condition 11 in FIG. 15); (iii) IL-2, IL-12, and IL-18 (condition 3 in FIG. 15); (iv) IL-12, IL-18 and IL-23 (condition 12 in FIG. 15); (v) IL-2, IL-12, IL-18 and IL-23 (condition 13 in FIG. 15), or (vi) IL-7, IL-15, IL-12 and IL-18 (condition 8 in FIG. 15).

Most preferably, the one or more interleukin may comprise a combination of IL-12, IL-18 and IL-23. Accordingly, preferably the stimulating step comprises contacting the PBMCs with (a) an antigen comprising either MR1/5-OP-RU or 5-OP-RU; and (b) a combination of IL-12, IL-18 and IL-23.

The inventors believe that they have devised a novel method for stimulating MAIT cells in a culture of PBMCs.

As such, in another aspect, there is provided a method of stimulating MAIT cells in a culture of PBMCs, the method comprising contacting a culture of PMBCs with (a) an antigen comprising MR1/5-OP-RU or 5-OP-RU; and/or (b) one or more interleukin selected from a group consisting of IL-2, IL-7, IL-12, IL-15, IL-18 and IL-23, or any combination thereof.

Preferably, the one or more interleukin is IL-12, IL-18 and/or IL-23. More preferably, the one or more interleukin is IL-12, IL-18 and IL-23.

The inventors believe that their method of producing CAR-MAIT cells is also novel per se.

Accordingly, in a third aspect, there is provided a method of producing a CAR-MAIT cell, the method comprising:
(i) providing peripheral blood monocyte cells (PBMCs);
(ii) subjecting the PBMCs to MACS and/or FACS to isolate MAIT cells therefrom;
(iii) activating the isolated MAIT cells, optionally by contacting them with an anti-CD3 and/or anti-CD28 antibody; and
(iv) transducing the activated MAIT cells with a nucleic acid encoding a CAR, to thereby produce a CAR-MAIT cell.

Steps (i), (ii) and/or (iii) of the method of the third aspect may be the same as the steps described herein in relation to the method of the second aspect, and so these method steps are interchangeable.

In addition, preferably the method comprises stimulating the PBMCs before they are subjected to the MACS and/or FACS step (i.e. step ii). Preferably, this initial stimulating step comprises contacting the PBMCs with (a) an antigen comprising either MR1/5-OP-RU or 5-OP-RU; and/or (b) a cytokine. Preferably, the stimulating step comprises contacting the PBMCs with (a) an antigen comprising either MR1/5-OP-RU or 5-OP-RU; and (b) a cytokine. The cytokine may be an interleukin as described in relation to the second aspect, preferably one or more interleukin selected from a group consisting of IL-2, IL-7, IL-12, IL-15, IL-18 and IL-23, or any combination thereof, as described above.

Most preferably, the one or more interleukin may comprise a combination of IL-12, IL-18 and IL-23. Accordingly, preferably the stimulating step comprises contacting the PBMCs with (a) an antigen comprising either MR1/5-OP-RU or 5-OP-RU; and (b) a combination of IL-12, IL-18 and IL-23.

It is preferred that the MAIT cells are activated in step (iii) before the isolated MAIT cells are transduced with the nucleic acid encoding the CAR in step (iv). The isolated MAIT cells may be activated with an anti-CD3 antibody, preferably in vitro. The isolated MAIT cells may be activated with an anti-CD28 antibody, preferably in vitro. Preferably, the isolated MAIT cells are activated with both anti-CD3 and anti-CD28 antibodies, either substantially simultaneously or sequentially, as described in relation to the method of the second aspect.

The MAIT cell transduction procedure (i.e. step (iv)) may comprise transducing the MAIT cells with a nucleic acid encoding a CAR. The transduction step may comprise viral transduction. Preferably, the MAIT cell transduction procedure comprises retrovirally transducing the MAIT cells with the nucleic acid encoding a CAR. The MAIT cell may be transduced with any nucleic acid encoding a CAR as described herein, for example according to the fifth or the vector according to the sixth aspect. The nucleic acid may encode a CAR which targets a CD4 antigen or at least one or more TCR Vbeta region on a T-cell. Preferably, the nucleic acid may encode a CAR which targets one or more TCR Vbeta region shown in Table 1 (and more preferably the TCR-Vbeta 7.1 chain) on a T-cell. The nucleic acid may encode a CAR which targets one or more TCR Vbeta region on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20.

Preferably, transduction is performed at least 34 hours, 36 hours or 48 hours after MAIT cell activation. Before transduction (e.g. about one day before), a RetroNectin coated plate may be prepared. RetroNectin (e.g. about 15 μg) may be contacted with PBS (e.g. about 1 ml) to form a solution. The method may comprise introducing solution to one well of the non-tissue culture treated 24-well-plate. The plate may be wrapped (e.g. with cling-film) and stored at about 4° C. (e.g. in a fridge over-night). On the day of gene transfer, unbound RetroNectin is removed from the well. The well may be washed (e.g. once or twice with 2 ml PBS). Preferably, the well is not allowed to dry. A retroviral supernatant may be thawed (e.g. in a 37° C. water bath). Viral supernatant is preferably transferred (e.g. about 1 ml) to each well of the RetroNectin-coated plate. The plate may be wrapped by cling-film. The plate may be centrifuged (e.g. at 1000×g at 32ºC for 2 hours). While centrifuging, activated MAIT cells may be collected. Collected cells may be resuspended (e.g. in fresh R10 medium containing 100 IU/ml IL-2 to concentration of 1×10⁶/ml). Once the centrifugation has completed, the supernatant may be discarded from the plate. Cell suspension (e.g. 1 ml) may be added to each well. The plate may be centrifuged (e.g. at 500×g for 10 min). The plate may then be incubated (e.g. in a 37ºC incubator). If required, the transduction step may be repeated to achieve higher transduction efficiency. Transduction efficiency may be detected about 48 hours after transduction by flow cytometry.

The method of the invention preferably comprises expanding the CAR-MAIT cells in a subsequent step after step (iv) in the method of the third aspect. The CAR-MAIT cell expansion step may comprise harvesting the transduced CAR-MAIT cells one or two days after transduction, preferably with a retrovirus or virus. The harvested cells may be counted (e.g. by a haemocytometer). Harvested cells may then be transferred to a well of a plate (e.g. 1×10⁷ cells to a well of a Grex6M well plate). The harvested cells may then be contacted with an interleukin. For example the interleukin may be IL-2 (e.g. about 100 IU/ml) contained in a suitable medium, such as R10 medium (e.g. 130 ml). The plate may be returned to the incubator. IL-2 may then be refreshed (e.g. to a final concentration of 100 IU/ml every three days). The CAR-MAIT cells may be harvested after 8-12 days culture. Expanded CAR-MAIT cells may be used for a phenotype test, a functional assay and/or be frozen for later use (e.g. in liquid nitrogen).

Advantageously, as described in the Examples, 11 days of culture yielded a 100-fold expansion level of CAR-MAIT cells with higher than 50% of transduction efficiency. The inventors observed that the CAR-MAIT cells surprisingly showed at least an equivalent cytotoxic potency to conventional CAR-expressing CD8+ T cells.

In a fourth aspect, there is provided a CAR-MAIT cell obtained, or obtainable, by the method of the third aspect.

As described herein, the isolated MAIT cell obtained using the method of either the second or third aspect, may be activated, and is ultimately transduced with a nucleic acid construct encoding the CAR to produce the CAR-MAIT cell of the first or fourth aspects. As discussed herein, the inventors have developed novel genetic constructs and recombinant vectors encoding a CAR, which specifically targets either the CD4 molecule (in which case the construct and vector is referred to herein as “CART4”) or one or more TCR-Vbeta region, such as the TCR-Vbeta 7.1 chain (in which case the construct and vector is referred to herein as “CARTVb7.1”) on T-cells. Any of these constructs and vectors may be used to transduce the MAIT cells in the method of the third or fourth aspects.

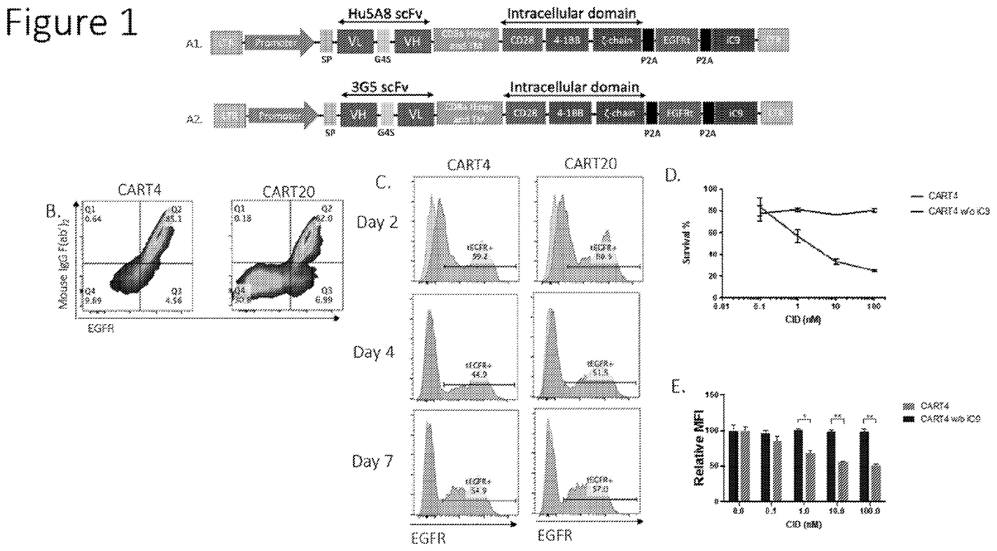

The genetic construct comprises the scFv of either (i) an anti-CD4 mAb (e.g. Hu5A8) or (ii) an anti-TCR-Vb mAb, for example an anti-TCR-Vb 7.1 mAb (e.g. 3G5), with CD28/4-1BB/CD3-zeta chain signalling moieties to form a third generation CAR. The construct encoding the CAR further comprises at least one safety switch encoded by a so-called suicide gene, such as truncated epidermal growth factor receptor (tEGFR) and/or inducible caspase-9 (iC9), and which enable the clearing of the resultant CAR-T cells as desired, and so provides an elegant monitoring system or safety mechanism when using the CAR-T cells in therapy. The expression of tEGFR can be recognised by anti-EGFR mAb (e.g. Cetuximab) for monitoring or depleting the CAR-T cells, and iC9 is a modified human Caspase-9 fused to human FK506 binding protein to allow conditional dimerization using a chemical inducer of dimerization (such as Rimiducid) which triggers apoptosis of the CAR-T cells expressing the fusion protein. The inventors believe that the CAR-encoding nucleic acid construct that they have developed is novel in its own right. Referring to FIG. 1A (1 and 2), there is shown schematic maps illustrating the functional elements included in two embodiments of a CAR-encoding construct according to the invention, i.e. “CART4” is shown in FIG. 1A(1), and “CARTVb7.1” is shown in FIG. 1A(2). It will be appreciated, however, that “CARTVb7.1” having anti-TCR-Vb 7.1 targeting the Vbeta 7.1 family is purely illustratative, and that any Vbeta region may be targeted by the CAR, for example any of the Vbetas shown in Table 1, and especially any of Vb 1, Vb 2, Vb 3, Vb 5.1, Vb 7.1, Vb 8, Vb 12, Vb 13.1, Vb 17, and Vb 20.

Hence, in a fifth aspect, there is provided a nucleic acid construct comprising a promoter operably linked to a first coding sequence, which encodes either an anti-CD4 chimeric antigen receptor (CAR) or an anti-T-cell receptor (TCR) V-beta CAR.

The promoter may be any suitable promoter, including a constitutive promoter, an activatable promoter, an inducible promoter, or a tissue-specific promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEFla), ubiquitin C promoter (UbiC), phosphoglycerokinase promoter (PGK), simian virus 40 early promoter (SV40), and chicken β-Actin promoter coupled with CMV early enhancer (CAGG). Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof. In some embodiments, the inducing condition does not induce the expression of endogenous genes in the engineered mammalian cell, and/or in the subject that receives the pharmaceutical composition. In some embodiments, the inducing 35 condition is selected from the group consisting of: inducer, irradiation (such as ionizing radiation, light), temperature (such as heat), redox state, tumor environment, and the activation state of the engineered mammalian cell.

In one embodiment, the promoter may be the PGK promoter (EMBL NO: A19297.1). In an embodiment, the PGK promoter is referred to herein as SEQ ID No:3, as follows:

[SEQ ID No: 3]
GGGTAGGGGAGGCGCTTTTCCCAAGGCAGTCTGGAGCATGCGCTTTAGC
AGCCCCGCTGGGCACTTGGCGCTACACAAGTGGCCTCTGGCCTCGCACA
CATTCCACATCCACCGGTAGGCGCCAACCGGCTCCGTTCTTTGGTGGCC
CCTTCGCGCCACCTTCTACTCCTCCCCTAGTCAGGAAGTTCCCCCCCGC
CCCGCAGCTCGCGTCGTGCAGGACGTGACAAATGGAAGTAGCACGTCTC
ACTAGTCTCGTGCAGATGGACAGCACCGCTGAGCAATGGAAGCGGGTAG
GCCTTTGGGGCAGCGGCCAATAGCAGCTTTGCTCCTTCGCTTTCTGGGC
TCAGAGGCTGGGAAGGGGTGGGTCCGGGGGCGGGCTCAGGGGGGGGCTC
AGGGGCGGGGGGGGCGCCCGAAGGTCCTCCGGAGGCCCGGCATTCTGCA
CGCTTCAAAAGCGCACGTCTGCCGCGCTGTTCTCCTCTTCCTCATTCTC
CGGGCCTTTCG

Preferably, therefore, the promoter may comprise a nucleotide sequence substantially as set out in SEQ ID No: 3, or a fragment or variant thereof.

The nucleic acid construct may comprise a nucleotide sequence encoding a signalling peptide. Advantageously, the signalling peptide is configured to lead the CAR (i.e. which is a fusion protein) to the T-cell outer membrane. Preferably, the sequence encoding the signalling peptide is disposed 3′ of the promoter. Preferably, the signalling peptide is an Igk signalling peptide. In one embodiment, the signalling peptide can have an amino acid sequence referred to herein as SEQ ID No:4, as follows:

[SEQ ID No: 4]
METDTLLLWVLLLWVPGSTGD

Preferably, therefore, the construct comprises a nucleotide sequence encoding a signalling peptide having an amino acid sequence substantially as set out in SEQ ID No:4, or a fragment or variant thereof.

In one embodiment, a nucleotide sequence encoding the signalling peptide is referred to herein as SEQ ID No:5, as follows:

[SEQ ID No: 5]
ATGGAGACAGACACACTCCTGCTATGGGTGCTGCTGCTCTGGGTTCCAG
GTTCCACAGGTGAC

Preferably, therefore, the construct comprises a nucleotide sequence substantially as set out in SEQ ID No: 5, or a fragment or variant thereof.

Preferably, the first coding sequence is disposed 3′ of the sequence encoding the signalling peptide. In a first embodiment of the nucleic acid construct, the first coding sequence encodes an anti-CD4 chimeric antigen receptor (CAR). Preferably, the CAR is specific for a CD4 antigen which comprises an amino acid sequence substantially as set out in SEQ ID No:1, or a variant or fragment thereof.

As shown in FIG. 1A(1) (“CART4”), the first coding sequence may encode a scFv region, which may comprise a VL (variable light chain) sequence and a VH (variable heavy chain) sequence. Preferably, the VL sequence is upstream (i.e. 5′) of the VH sequence. In some embodiments, however, the VH sequence may be upstream of the VL sequence.

The VL and VH sequences may, in one embodiment, be a Hu5A8 (i.e. the hybridoma clone name of an anti-CD4 monoclonal antibody) light chain variable region and heavy chain variable region for binding CD4 antigen on a T-cell.

Thus, in one embodiment, the first coding sequence (which may encode a VL sequence for binding CD4) encodes an amino acid sequence referred to herein as SEQ ID No:6, as follows:

[SEQ ID No: 6]
DIVMTQSPDSLAVSLGERVTMNCKSSQSLLYSTNQKNYLAWYQQKPGQS
PKLLIYWASTRESGVPDRFSGSGSGTDFTLTISSVQAEDVAVYYCQQYY
SYRTFGGGTKLEIK

Preferably, therefore, the first coding sequence comprises a nucleotide sequence encoding an amino acid sequence substantially as set out in SEQ ID No:6, or a fragment or variant thereof.

In one embodiment, the first coding sequence (which may encode a VL sequence for binding CD4) comprises a nucleotide sequence which is referred to herein as SEQ ID No:7, as follows:

[SEQ ID No: 7]
GACATTGTGATGACTCAGAGCCCCGACAGCCTGGCCGTCTCACTGGGCG
AAAGGGTGACCATGAATTGTAAATCTTCTCAGAGCCTGCTGTACAGTAC
AAACCAGAAAAATTACCTGGCCTGGTATCAGCAGAAACCCGGCCAGAGC
CCTAAGCTGCTGATCTATTGGGCAAGTACCCGAGAGTCAGGAGTGCCAG
ACAGATTCTCCGGGTCTGGAAGTGGCACAGACTTCACCCTGACAATTAG
CTCCGTGCAGGCCGAGGACGTGGCTGTCTACTATTGCCAGCAGTACTAT
AGCTACCGAACTTTCGGCGGGGGAACCAAACTGGAAATCAAG

Preferably, therefore, the first coding sequence comprises a nucleotide sequence substantially as set out in SEQ ID No: 7, or a fragment or variant thereof.

In one embodiment, the first coding sequence (which may encode a VH sequence for binding CD4) encodes an amino acid sequence referred to herein as SEQ ID No:8, as follows:

[SEQ ID No: 8]
QVQLQQSGPEVVKPGASVKMSCKASGYTFTSYVIHWVRQKPGQGLDWIG
YINPYNDGTDYDEKFKGKATLTSDTSTSTAYMELSSLRSEDTAVYYCAR
EKDNYATGAWFAYWGQGTLVTVSS

Preferably, therefore, the first coding sequence comprises a nucleotide sequence encoding an amino acid sequence substantially as set out in SEQ ID No:8, or a fragment or variant thereof.

In one embodiment, the first coding sequence (which may encode a VH sequence for binding CD4) comprises a nucleotide sequence which is referred to herein as SEQ ID No:9, as follows:

[SEQ ID No: 9]
CAGGTGCAGCTGCAGCAGTCCGGACCAGAGGTGGTCAAACCCGGCGCTA
GCGTCAAAATGTCCTGTAAGGCATCTGGCTACACTTTCACCTCTTATGT
GATTCACTGGGTCAGACAGAAGCCTGGGCAGGGACTGGACTGGATCGGG
TACATTAACCCATATAATGATGGAACTGACTACGATGAAAAGTTTAAAG
GCAAGGCCACACTGACTTCCGACACCTCAACAAGCACTGCTTATATGGA
GCTGTCTAGTCTGAGGTCTGAAGACACAGCAGTGTACTATTGCGCCCGC
GAGAAGGATAACTACGCCACTGGCGCTTGGTTTGCATATTGGGGCCAGG
GGACCCTGGTGACAGTCTCATCC

Preferably, therefore, the first coding sequence comprises a nucleotide sequence substantially as set out in SEQ ID No: 9, or a fragment or variant thereof.

Preferably, the VH (e.g. SEQ ID No: 9) and VL (e.g. SEQ ID No: 7) sequences, when in either orientation, are separated by a linker sequence. In an embodiment, the linker sequence may be a G4S linker sequence, which is referred to herein as SEQ ID No: 10, as follows:

[SEQ ID No: 10]
GGGGSGGGGSGGGGS

Preferably, therefore, the first coding sequence comprises a nucleotide sequence encoding an amino acid sequence substantially as set out in SEQ ID No: 10, or a fragment or variant thereof.

In one embodiment, the linker sequence can be encoded by a nucleotide sequence, which is referred to herein as SEQ ID No:11, as follows:

[SEQ ID No: 11]
GAGGAGGAGGCAGTGGCGGAGGAGGGTCAGGAGGAGGAGGAAGC

Preferably, therefore, the linker sequence comprises a nucleotide sequence substantially as set out in SEQ ID No: 11, or a fragment or variant thereof.

However, in a second embodiment of the nucleic acid construct (“CARTVb7.1”), the first coding sequence encodes an anti-T-cell receptor (TCR) V-beta region CAR. It will be appreciated that any Vbeta region may be targeted by the CAR. For example, any of the Vbeta regions listed in Table 1 may be targeted by the CAR which is encoded by the first coding sequence.

The first coding sequence may encode a plurality of T-cell receptor (TCR) beta-chain variable regions (Vbeta) CARs. Preferably, the plurality of Vbeta regions may be selected from a group of Vbeta regions shown in Table 1. It is also possible to combine two or more Vbeta region-targeting CARs on the same construct. For example, the construct may comprise coding sequences which encode at least two, or at least three or at least four T-cell receptor (TCR) beta-chain variable region (Vbeta)-targeting CARs, preferably as listed in Table 1. The construct may comprise coding sequence which encodes at least five, or at least six or at least seven T-cell receptor (TCR) beta-chain variable region (Vbeta)-targeting CARs, preferably as listed in Table 1. The plurality of TCR V beta regions may be the same or different V beta regions.

The following Vb families are believed to be frequently associated with T-cell lymphoma, i.e. Vb 1, Vb 2, Vb 3, Vb 5.1, Vb 7.1, Vb 8, Vb 12, Vb 13.1, Vb 17, and Vb 20. Therefore, in a preferred embodiment, the construct comprises a coding sequence encoding at least one CAR which targets one or more TCR Vbeta region on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20. Preferably, the construct comprises a coding sequence encoding at least one CAR which targets at least two or three TCR Vbeta regions on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20.

Accordingly, preferably there is provided a nucleic acid construct comprising a promoter operably linked to a first coding sequence, which encodes a plurality of anti-T-cell receptor (TCR) V-beta CARs, wherein different V-beta regions on a T-cell are targeted.

Preferably, the CAR is specific for a TCR Vbeta region (preferably, TCR-Vbeta 7.1 chain) which comprises an amino acid sequence substantially as set out in SEQ ID No:2, or a variant or fragment thereof.

As shown in FIG. 1A(2), the first coding sequence may encode a scFv region, which may comprise a VL (variable light chain) sequence and a VH (variable heavy chain) sequence. Preferably, the VL sequence is upstream (i.e. 5′) of the VH sequence. In some embodiments, however, the VH sequence may be upstream of the VL sequence. Preferably, the VH and VL encoding sequences, in either orientation, are separated by a linker sequence, such as a G4S linker sequence.

The VL and VH sequences may, in one embodiment, be a 3G5 (i.e. the hybridoma clone name of an anti-TCR V beta 7.1 monoclonal antibody) light chain variable region and heavy chain variable region for binding TCR V-beta 7.1 antigen.

In one embodiment, the first coding sequence (which may encode a VL sequence for binding TCR V-beta, preferably TCR-Vbeta 7.1 chain) encodes an amino acid sequence referred to herein as SEQ ID No:12, as follows:

[SEQ ID No: 12]
QVQLQQPGAELVKPGASVKMSCKASGYTFTRYWITWVKQRPGQGL
EWIGDIYPGSGFTKYNEKFKSKATLTVDTSSSTAYMQLSSLTSED
SAVYYCAREGGNYWYFDVWGTGTTVTVSS

Preferably, therefore, the first coding sequence comprises a nucleotide sequence encoding an amino acid sequence substantially as set out in SEQ ID No: 12, or a fragment or variant thereof.

In one embodiment, the first coding sequence (which may encode a VL sequence for binding TCR V-beta, preferably TCR-Vbeta 7.1 chain) comprises a nucleotide sequence which is referred to herein as SEQ ID No: 13, as follows:

[SEQ ID No: 13]
CAAGTTCAGCTGCAACAGCCTGGCGCCGAGCTTGTGAAACCTGGC
GCCTCTGTGAAGATGAGCTGCAAGGCCTCCGGCTACACCTTCACC
AGATACTGGATCACCTGGGTCAAGCAGAGGCCTGGACAGGGACTC
GAGTGGATCGGCGATATCTATCCTGGCTCCGGCTTCACCAAGTAC
AACGAGAAGTTCAAGAGCAAGGCCACACTGACCGTGGACACCAGC
AGCAGCACAGCCTACATGCAGCTGTCTAGCCTGACCAGCGAGGAC
AGCGCCGTGTACTACTGTGCTAGAGAAGGCGGCAACTACTGGTAC
TTCGACGTGTGGGGCACCGGCACCACAGTGACAGTTAGTTCT

Preferably, therefore, the first coding sequence comprises a nucleotide sequence substantially as set out in SEQ ID No: 13, or a fragment or variant thereof.

In one embodiment, the first coding sequence (which may encode a VH sequence for binding TCR V-beta, preferably TCR-Vbeta 7.1 chain) encodes an amino acid sequence referred to herein as SEQ ID No:34, as follows:

[SEQ ID No: 34]
DIQMTQSPSSLSASLGGKVTLTCKASQDINKYIAWYQHKPGKGPR
LLIHYTSTLQPGIPSRFSGSGSGRDYSFSISNLEPEDVATYYCLQ
YDNLRTFGGGTKLEIKRTD

Preferably, therefore, the first coding sequence comprises a nucleotide sequence encoding an amino acid sequence substantially as set out in SEQ ID No: 34, or a fragment or variant thereof.

In one embodiment, the first coding sequence (which may encode a VH sequence for binding TCR V-beta, preferably TCR-Vbeta 7.1 chain) comprises a nucleotide sequence which is referred to herein as SEQ ID No: 35, as follows:

[SEQ ID No: 35]
GACATCCAGATGACACAGAGCCCTAGCAGCCTGTCTGCCTCTCTC
GGCGGAAAAGTGACCCTGACATGCAAGGCCAGCCAGGACATCAAC
AAGTATATCGCCTGGTATCAGCACAAGCCCGGCAAGGGACCTAGA
CTGCTGATCCACTACACCAGCACACTGCAGCCTGGCATCCCCAGC
AGATTTTCTGGCAGCGGCTCCGGCAGAGACTACAGCTTCAGCATC
AGCAACCTGGAACCTGAGGACGTGGCCACCTACTACTGCCTGCAG
TACGACAACCTGCGGACCTTTGGCGGCGGAACAAAGCTGGAAATC
AAGCGGACAGAT

Preferably, therefore, the first coding sequence comprises a nucleotide sequence substantially as set out in SEQ ID No: 35, or a fragment or variant thereof.

Preferably, the VH (e.g. SEQ ID No: 35) and VL (e.g. SEQ ID No: 13) sequences, when in either orientation, are separated by a linker sequence. In an embodiment, the linker sequence may be a G4S linker sequence, which may comprise or consist of an amino acid sequence substantially as set out in SEQ ID No: 10, or a fragment or variant thereof. Preferably, therefore, the linker sequence comprises a nucleotide sequence substantially as set out in SEQ ID No: 11, or a fragment or variant thereof.

The nucleic acid construct may comprise a nucleotide sequence encoding a CD8a hinge and transmembrane (TM) structure domain. Advantageously, the hinge and TM domain are configured for CAR display and anchoring on the CAR-T cell. Preferably, the sequence encoding the hinge and TM domain is disposed 3′ of the first coding sequence.

In one embodiment, the amino acid sequence of a CD8a hinge and transmembrane domain is referred to herein as SEQ ID No: 14, as follows:

[SEQ ID No: 14]
FVPVFLPAKPTTTPAPRPPTPAPTIASQPLSLRPEACRPAAGGAV
HTRGLDFACDIYIWAPLAGTCGVLLLSLVITLYCNHRN

Preferably, therefore, the construct comprises a nucleotide sequence encoding an amino acid sequence substantially as set out in SEQ ID No: 14, or a fragment or variant thereof.

In an embodiment, a nucleotide sequence encoding the CD8a hinge and transmembrane domain is referred to herein as SEQ ID No: 15, as follows:

[SEQ ID No: 15]
TTCGTGCCGGTCTTCCTGCCAGCGAAGCCCACCACGACGCCAGCG
CCGCGACCACCAACACCGGCGCCCACCATCGCGTCGCAGCCCCTG
TCCCTGCGCCCAGAGGCGTGCCGGCCAGCGGCGGGGGGCGCAGTG
CACACGAGGGGGCTGGACTTCGCCTGTGATATCTACATCTGGGCG
CCCTTGGCCGGGACTTGTGGGGTCCTTCTCCTGTCACTGGTTATC
ACCCTTTACTGCAACCACAGGAAC

Preferably, therefore, the construct comprises a nucleotide sequence substantially as set out in SEQ ID No: 15, or a fragment or variant thereof.

The nucleic acid construct may comprise a nucleotide sequence encoding an intracellular domain, which may comprise a signalling domain of CD28, a signalling domain of 4-1BB and/or a CD3ζ chain, and more preferably a signalling domain of CD28, a signalling domain of 4-1BB and a CD3ζ chain. It will be appreciated that these components form the basis of third generation CAR and are required for triggering the intracellular signalling pathway. Preferably, the intracellular domain is disposed 3′ of the sequence encoding the hinge and transmembrane domain. The signalling domain of CD28 may be 5′ of the signalling domain of 4-1BB. The signalling domain of 4-1BB may be 5′ of the CD3ζ chain.

One embodiment of the CD28 signalling domain can have an amino acid sequence, which is referred to herein as SEQ ID No: 16, as follows:

[SEQ ID No: 16]
RSKRSRLLHSDYMNMTPRRPGPTRKHYQPYAPPRDFAAYRS

Preferably, therefore, the construct comprises a nucleotide sequence encoding an amino acid sequence substantially as set out in SEQ ID No: 16, or a fragment or variant thereof.

In an embodiment, the CD28 signalling domain can be encoded by a nucleic acid sequence, which is referred to herein as SEQ ID No: 17, as follows:

[SEQ ID No: 17]
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCC

Preferably, therefore, the construct comprises a nucleotide sequence substantially as set out in SEQ ID No: 17, or a fragment or variant thereof.

One embodiment of the 4-1BB signalling domain can have an amino acid sequence, which is referred to herein as SEQ ID No: 18, as follows:

[SEQ ID No: 18]
RFSVVKRGRKKLLYIFKQPFMRPVQTTQEEDGCSCRFPEEEEGGC
EL

Preferably, therefore, the construct comprises a nucleotide sequence encoding an amino acid sequence substantially as set out in SEQ ID No: 18, or a fragment or variant thereof.

In an embodiment, the 4-1BB signalling domain can be encoded by a nucleic acid sequence, which is referred to herein as SEQ ID No: 19, as follows:

[SEQ ID No: 19]
CGTTTCTCTGTTGTTAAACGGGGCAGAAAGAAGCTCCTGTATATA
TTCAAACAACCATTTATGAGACCAGTACAAACTACTCAAGAGGAA
GATGGCTGTAGCTGCCGATTTCCAGAAGAAGAAGAAGGAGGATGT
GAACTG

Preferably, therefore, the construct comprises a nucleotide sequence substantially as set out in SEQ ID No: 19, or a fragment or variant thereof.

One embodiment of the CD3ζ chain can have an amino acid sequence, which is referred to herein as SEQ ID No: 20, as follows:

[SEQ ID No: 20]
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

Preferably, therefore, the construct comprises a nucleotide sequence encoding an amino acid sequence substantially as set out in SEQ ID No: 20, or a fragment or variant thereof.

In an embodiment, the CD3ζ chain can be encoded by a nucleic acid sequence, which is referred to herein as SEQ ID No: 21, as follows:

[SEQ ID No: 21]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

Preferably, therefore, the construct comprises a nucleotide sequence substantially as set out in SEQ ID No: 21, or a fragment or variant thereof.

Preferably, the nucleic acid construct comprises a second coding sequence, which encodes at least one suicide protein, and more preferably at least two suicide proteins. The construct of the invention is advantageous in that the presence of the second coding sequence encoding the at least one suicide protein means that resulting CAR-T cells transduced with the construct can be controllably or inducibly detected or eliminated, for example in the case of an adverse patient reaction.

Preferably, therefore, in one embodiment, there is provided a nucleic acid construct comprising a promoter operably linked to a first coding sequence, which encodes an anti-CD4 chimeric antigen receptor (CAR), and a second coding sequence, which encodes at least one suicide protein, and more preferably at least two suicide proteins.

Preferably, in another embodiment, there is provided a nucleic acid construct comprising a promoter operably linked to a first coding sequence, which encodes an anti-T-cell receptor (TCR) V-beta CAR, and a second coding sequence, which encodes at least one suicide protein, and more preferably at least two suicide proteins.

In yet another embodiment, preferably there is provided a nucleic acid construct comprising a promoter operably linked to a first coding sequence, which encodes a plurality of anti-T-cell receptor (TCR) V-beta CARs, wherein different V-beta regions on a T-cell are targeted, and a second coding sequence, which encodes at least one suicide protein, and more preferably at least two suicide proteins.

In one embodiment, the second coding sequence may encode epidermal growth factor receptor (EGFR), or truncated epidermal growth factor receptor (tEGFR) (UniProt No. P00533; NCBI reference sequence NP 001333826.1). The expression of tEGFR can be controlled by anti-EGFR mAb (Cetuximab) for monitoring or depletion of the CAR-T cells in a patient. In one embodiment, the amino acid sequence of tEGFR may referred to herein as SEQ ID No: 22, as follows:

[SEQ ID No: 22]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKOHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFM

Preferably, therefore, the construct comprises a nucleotide sequence encoding an amino acid sequence substantially as set out in SEQ ID No: 22, or a fragment or variant thereof.

In an embodiment, tEGFR can be encoded by a nucleic acid sequence, which is referred to herein as SEQ ID No: 23, as follows:

[SEQ ID No: 23]
ATGCTTCTCCTGGTGACAAGCCTTCTGCTCTGTGAGTTACCACACCCAGC
ATTCCTCCTGATCCCACGCAAAGTGTGTAACGGAATAGGTATTGGTGAAT
TTAAAGACTCACTCTCCATAAATGCTACGAATATTAAACACTTCAAAAAC
TGCACCTCCATCAGTGGCGATCTCCACATCCTGCCGGTGGCATTTAGGGG
TGACTCCTTCACACATACTCCTCCTCTGGATCCACAGGAACTGGATATTC
TGAAAACCGTAAAGGAAATCACAGGGTTTTTGCTGATTCAGGCTTGGCCT
GAAAACAGGACGGACCTCCATGCCTTTGAGAACCTAGAAATCATACGCGG
CAGGACCAAGCAACATGGTCAGTTTTCTCTTGCAGTCGTCAGCCTGAACA
TAACATCCTTGGGATTACGCTCCCTCAAGGAGATAAGTGATGGAGATGTG
ATAATTTCAGGAAACAAAAATTTGTGCTATGCAAATACAATAAACTGGAA
AAAACTGTTTGGGACCTCCGGTCAGAAAACCAAAATTATAAGCAACAGAG
GTGAAAACAGCTGCAAGGCCACAGGCCAGGTCTGCCATGCCTTGTGCTCC
CCCGAGGGCTGCTGGGGCCCGGAACCCAGGGACTGCGTCTCTTGCCGGAA
TGTCAGCCGAGGCAGGGAATGCGTGGACAAGTGCAACCTTCTGGAGGGTG
AGCCAAGGGAGTTTGTGGAGAACTCTGAGTGCATACAGTGCCACCCAGAG
TGCCTGCCTCAGGCCATGAACATCACCTGCACAGGACGGGGACCAGACAA
CTGTATCCAGTGTGCCCACTACATTGACGGCCCCCACTGCGTCAAGACCT
GCCCGGCAGGAGTCATGGGAGAAAACAACACCCTGGTCTGGAAGTACGCA
GACGCCGGCCATGTGTGCCACCTGTGCCATCCAAACTGCACCTACGGATG
CACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCC
CTGGGGATCGGCCTCTTCATG

Preferably, therefore, the construct comprises a nucleotide sequence substantially as set out in SEQ ID No: 23, or a fragment or variant thereof.

In another embodiment, the second coding sequence may encode inducible caspase-9 (iC9). iC9 is a modified human Caspase-9 (UniProt No. P55211; NCBI reference sequence NP 001220.2) fused to the human FK506 binding protein (UniProt No. P62942; NCBI reference sequence NP 000792.1) to allow conditional dimerization using a chemical inducer of dimerization (caspase inducible drug (CID), Rimiducid), which triggers apoptosis of the CAR-T cells expressing the fusion protein. In one embodiment, the amino acid sequence of iC9 may be referred to herein as SEQ ID No: 24, as follows:

[SEQ ID No: 24]
MLEGVQVETISPGDGRTFPKRGQTCVVHYTGMLEDGKKVDSSRDRNKPFK
FMLGKQEVIRGWEEGVAQMSVGORAKLTISPDYAYGATGHPGIIPPHATL
VFDVELLKLESGGGSGVDGFGDVGALESLRGNADLAYILSMEPCGHCLII
NNVNFCRESGLRTRTGSNIDCEKLRRRFSSLHFMVEVKGDLTAKKMVLAL
LELAQQDHGALDCCVVVILSHGCQASHLQFPGAVYGTDGCPVSVEKIVNI
FNGTSCPSLGGKPKLFFIQACGGEQKDHGFEVASTSPEDESPGSNPEPDA
TPFQEGLRTFDOLDAISSLPTPSDIFVSYSTFPGFVSWRDPKSGSWYVET
LDDIFEQWAHSEDLQSLLLRVANAVSVKGIYKQMPGCFNFLRKKLFFKTS
VDYPYDVPDYALD*

Preferably, therefore, the construct comprises a nucleotide sequence encoding an amino acid sequence substantially as set out in SEQ ID No: 24, or a fragment or variant thereof.

In an embodiment, iC9 can be encoded by a nucleic acid sequence, which is referred to herein as SEQ ID No: 25, as follows:

[SEQ ID No: 25]
ATGCTCGAGGGAGTGCAGGTGGAAACCATCTCCCCAGGAGACGGGCGCAC
CTTCCCCAAGCGCGGCCAGACCTGCGTGGTGCACTACACCGGGATGCTTG
AAGATGGAAAGAAAGTTGATTCCTCCCGGGACAGAAACAAGCCCTTTAAG
TTTATGCTAGGCAAGCAGGAGGTGATCCGAGGCTGGGAAGAAGGGGTTGC
CCAGATGAGTGTGGGTCAGAGAGCCAAACTGACTATATCTCCAGATTATG
CCTATGGTGCCACTGGGCACCCAGGCATCATCCCACCACATGCCACTCTC
GTCTTCGATGTGGAGCTTCTAAAACTGGAATCTGGCGGTGGATCCGGAGT
CGACGGATTTGGTGATGTCGGTGCTCTTGAGAGTTTGAGGGGAAATGCAG
ATTTGGCTTACATCCTGAGCATGGAGCCCTGTGGCCACTGCCTCATTATC
AACAATGTGAACTTCTGCCGTGAGTCCGGGCTCCGCACCCGCACTGGCTC
CAACATCGACTGTGAGAAGTTGCGGCGTCGCTTCTCCTCGCTGCATTTCA
TGGTGGAGGTGAAGGGCGACCTGACTGCCAAGAAAATGGTGCTGGCTTTG
CTGGAGCTGGCGCAGCAGGACCACGGTGCTCTGGACTGCTGCGTGGTGGT
CATTCTCTCTCACGGCTGTCAGGCCAGCCACCTGCAGTTCCCAGGGGCTG
TCTACGGCACAGATGGATGCCCTGTGTCGGTCGAGAAGATTGTGAACATC
TTCAATGGGACCAGCTGCCCCAGCCTGGGAGGGAAGCCCAAGCTCTTTTT
CATCCAGGCCTGTGGTGGGGAGCAGAAAGACCATGGGTTTGAGGTGGCCT
CCACTTCCCCTGAAGACGAGTCCCCTGGCAGTAACCCCGAGCCAGATGCC
ACCCCGTTCCAGGAAGGTTTGAGGACCTTCGACCAGCTGGACGCCATATC
TAGTTTGCCCACACCCAGTGACATCTTTGTGTCCTACTCTACTTTCCCAG
GTTTTGTTTCCTGGAGGGACCCCAAGAGTGGCTCCTGGTACGTTGAGACC
CTGGACGACATCTTTGAGCAGTGGGCTCACTCTGAAGACCTGCAGTCCCT
CCTGCTTAGGGTCGCTAATGCTGTTTCGGTGAAAGGGATTTATAAACAGA
TGCCTGGTTGCTTTAATTTCCTCCGGAAAAAACTTTTCTTTAAAACATCA
GTCGACTATCCGTACGACGTACCAGACTACGCACTCGACTAA

Preferably, therefore, the construct comprises a nucleotide sequence substantially as set out in SEQ ID No: 25, or a fragment or variant thereof.

Preferably, the nucleic acid construct of the invention comprises a coding sequence encoding EGFR or truncated epidermal growth factor receptor (tEGFR) and/or inducible caspase-9 (iC9). Having one suicide gene provides robust control (for detection and/or elimination) on the expression of the CAR, and CAR-MAIT cells, when used in therapy.

More preferably, however, the nucleic acid construct comprises a coding sequence encoding both the truncated epidermal growth factor receptor (tEGFR) and inducible caspase-9 (iC9). Advantageously, having two suicide genes provides even tighter control (for detection and/or elimination) on the expression of the CAR, and CAR-MAIT cells, when used in therapy.

Preferably, the nucleic acid construct comprises a nucleotide sequence encoding a peptide spacer that is configured to be digested (or self-cleaved) to thereby produce encoded polypeptides either side of the spacer as separate molecules, for example the intracellular domains and the suicide gene-encoded protein, which may be tEGFR and/or iC9. Hence, the peptide spacer may be known as a self-cleaving peptide.

Preferably, the spacer sequence comprises and encodes a viral peptide spacer sequence, more preferably a viral 2A peptide spacer sequence (Furler S, Paterna J-C, Weibel M and Bueler H Recombinant AAV vectors containing the foot and mouth disease virus 2A sequence confer efficient bicistronic gene expression in cultured cells and rat substantia nigra neurons Gene Ther. 2001, vol. 8, PP: 864-873).

Preferably, the spacer sequence encoding the 2A peptide sequence connects the sequence encoding the intracellular domain to the sequence encoding the suicide protein. In embodiments in which the construct encodes two suicide proteins (e.g. EGFR/tEGFR and iC9), the nucleic acid construct comprises a first spacer sequence disposed between the sequence encoding the intracellular domain and the sequence encoding the first suicide protein, and a second spacer sequence disposed between the sequence encoding the first suicide protein and the sequence encoding the second suicide protein. The first suicide protein may be EGFR/tEGFR, and the second suicide protein may be iC9. In another embodiment, the first suicide protein may be iC9 and the second suicide protein may be EGFR/tEGFR.

The 2A spacer sequence may be any known variant, which includes those sequences referred to as E2A, F2A, P2A and T2A, as disclosed in Wang Y et al. Scientific Reports 2015, 5. Preferably, the self-cleaving peptide is a P2A. In an embodiment, the P2A spacer has an amino acid sequence, referred to herein as SEQ ID No: 26, as follows:

| [SEQ ID No: 26] | |
|---|---|
| | GSGATNFSLLKQAGDVEENPGP |

Preferably, therefore, the construct comprises a nucleotide sequence encoding an amino acid sequence substantially as set out in SEQ ID No: 26, or a fragment or variant thereof.

In an embodiment, the 2A spacer can be encoded by a nucleic acid sequence, which is referred to herein as SEQ ID No: 27, as follows:

GGATCCGGAGCCACGAACTTCTCTCTGTTAAAGCAAGCAGGAGACGTGGA
AGAAAACCCCGGTCCT

Preferably, therefore, the construct comprises a nucleotide sequence substantially as set out in SEQ ID No: 27, or a fragment or variant thereof.

In further embodiments, the construct may further comprise a nucleotide sequence encoding Woodchuck Hepatitis Virus Post-transcriptional Regulatory Element (WPRE), which enhances the expression of the transgenes. Preferably, the WPRE coding sequence is disposed 3′ of the sequence encoding the suicide protein. In particular, the WPRE sequence is preferably 3′ of the iC9-encoding sequence.

One embodiment of the WPRE is 592 bp long, including gamma-alpha-beta elements, and is referred to herein as SEQ ID No: 28, as follows:

[SEQ ID NO: 28]
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAGCTGA
CGTCCTTTCCATGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCCCCGCCTG

Preferably, the nucleic acid comprises a nucleic acid sequence substantially as set out in SEQ ID No: 28, or a fragment or variant thereof.

Preferably, the construct comprises left (i.e. 5′) and/or right (i.e. 3′) Long Terminal Repeat sequences (LTRs). Preferably, each LTR is disposed at the 5′ and/or 3′ end of the construct.

In a preferred embodiment, the nucleic acid construct comprises, in this specified order, a 5′ promoter; a sequence encoding scFv specific for CD4 or TCR V-beta region; and a 3′ sequence encoding an intracellular domain. The use of 5′ and 3′ indicates that the features are either upstream or downstream, and is not intended to indicate that the features are necessarily terminal features.

In a preferred embodiment, the nucleic acid construct comprises, in this specified order, a 5′ promoter; a sequence encoding scFv specific for CD4 or TCR V-beta region; a sequence encoding an intracellular domain; and a 3′ sequence encoding at least one suicide protein.

In a more preferred embodiment, the nucleic acid construct comprises, in this specified order, a 5′ promoter (preferably, PGK promoter); a sequence encoding scFv specific for CD4 or one or more TCR V-beta region (preferably, VL and/or VH domains); a sequence encoding an intracellular domain (preferably, CD28, 4-1BB and/or CD3ζ chain); a 3′ sequence encoding at least one suicide protein (preferably, EGFR/EGFRt and/or iC9).

In a yet more preferred embodiment, the nucleic acid construct comprises, in this specified order, a 5′ promoter (preferably, PGK promoter); a sequence encoding a signalling peptide (SP); a sequence encoding scFv specific for CD4 or one or more TCR V-beta region (preferably, VL and VH domains separated by a G4S linker); a sequence encoding a CD8a hinge and transmembrane domain; a sequence encoding an intracellular domain (preferably, CD28, 4-1BB and CD3ζ chain); a 3′ sequence encoding at least one suicide protein (preferably, EGFR/EGFRt and iC9), optionally with a self-cleaving peptide spacer between the sequences encoding the intracellular domain and suicide protein-encoding sequences.

In a first most preferred embodiment (known as “CART4”), the nucleic acid construct comprises, in this specified order, a 5′ PGK promoter; a sequence encoding a signalling peptide (SP); a sequence encoding VL and VH domains of a scFv specific for CD4 (preferably, separated by a G4S linker); a sequence encoding a CD8a hinge and transmembrane domain; a sequence encoding CD28, 4-1BB and CD3ζ chain of an intracellular domain; a sequence encoding a first self-cleaving peptide spacer; a sequence encoding EGFR/EGFRt; a sequence encoding a second self-cleaving peptide spacer; a 3′ sequence encoding iC9.

In a second most preferred embodiment (known as “CARTVb7.1”), the nucleic acid construct comprises, in this specified order, a 5′ PGK promoter; a sequence encoding a signalling peptide (SP); a sequence encoding VL and VH domains of a scFv specific for one or more TCR V-beta region, preferably TCR-Vbeta 7.1 chain (preferably, separated by a G4S linker); a sequence encoding a CD8a hinge and transmembrane domain; a sequence encoding CD28, 4-1BB and CD3ζ chain of an intracellular domain; a sequence encoding a first self-cleaving peptide spacer; a sequence encoding EGFR/EGFRt; a sequence encoding a second self-cleaving peptide spacer; a 3′ sequence encoding iC9.

Hence, one preferred embodiment of the nucleic acid construct (known as “CART4”) has an amino acid sequence, referred to herein as SEQ ID No: 29, as follows:

[SEQ ID No: 29]
METDTLLLWVLLLWVPGSTGDDIVMTQSPDSLAVSLGERVTMNCKSSQSL
LYSTNQKNYLAWYQQKPGQSPKLLIYWASTRESGVPDRFSGSGSGTDFTL
TISSVQAEDVAVYYCQQYYSYRTFGGGTKLEIKGGGGSGGGGSGGGGSQV
QLQQSGPEVVKPGASVKMSCKASGYTFTSYVIHWVRQKPGQGLDWIGYIN
PYNDGTDYDEKFKGKATLTSDTSTSTAYMELSSLRSEDTAVYYCAREKDN
YATGAWFAYWGQGTLVTVSSAAAFVPVFLPAKPTTTPAPRPPTPAPTIAS
QPLSLRPEACRPAAGGAVHTRGLDFACDIYIWAPLAGTCGVLLLSLVITL
YCNHRNRSKRSRLLHSDYMNMTPRRPGPTRKHYQPYAPPRDFAAYRSRFS
VVKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHM
QALPPRGSGATNFSLLKQAGDVEENPGPMLLLVTSLLLCELPHPAFLLIP
RKVCNGIGIGEFKDSLSINATNIKHFKNCTSISGDLHILPVAFRGDSFTH
TPPLDPQELDILKTVKEITGELLIQAWPENRTDLHAFENLEIIRGRTKQH
GQFSLAVVSLNITSLGLRSLKEISDGDVIISGNKNLCYANTINWKKLFGT
SGQKTKIISNRGENSCKATGQVCHALCSPEGCWGPEPRDCVSCRNVSRGR
ECVDKCNLLEGEPREFVENSECIQCHPECLPQAMNITCTGRGPDNCIQCA
HYIDGPHCVKTCPAGVMGENNTLVWKYADAGHVCHLCHPNCTYGCTGPGL
EGCPTNGPKIPSIATGMVGALLLLLVVALGIGLFMGSGATNFSLLKQAGD
VEENPGPMLEGVQVETISPGDGRTFPKRGQTCVVHYTGMLEDGKKVDSSR
DRNKPFKFMLGKQEVIRGWEEGVAQMSVGQRAKLTISPDYAYGATGHPGI
IPPHATLVFDVELLKLESGGGSGVDGFGDVGALESLRGNADLAYILSMEP
CGHCLIINNVNFCRESGLRTRTGSNIDCEKLRRRFSSLHFMVEVKGDLTA
KKMVLALLELAQQDHGALDCCVVVILSHGCQASHLQFPGAVYGTDGCPVS
VEKIVNIFNGTSCPSLGGKPKLFFIQACGGEQKDHGFEVASTSPEDESPG
SNPEPDATPFQEGLRTFDOLDAISSLPTPSDIFVSYSTFPGFVSWRDPKS
GSWYVETLDDIFEQWAHSEDLQSLLLRVANAVSVKGIYKOMPGCFNFLRK
KLFFKTSVDYPYDVPDYALD*

Preferably, therefore, the construct comprises a nucleotide sequence encoding an amino acid sequence substantially as set out in SEQ ID No: 29, or a fragment or variant thereof.

Preferably, the embodiment of the nucleic acid construct (known as “CART4”) has a nucleotide sequence, referred to herein as SEQ ID No: 30, as follows:

[SEQ ID No: 30]
ATGGAGACAGACACACTCCTGCTATGGGTGCTGCTGCTCTGGGTTCCAGG
TTCCACAGGTGACGACATTGTGATGACTCAGAGCCCCGACAGCCTGGCCG
TCTCACTGGGCGAAAGGGTGACCATGAATTGTAAATCTTCTCAGAGCCTG
CTGTACAGTACAAACCAGAAAAATTACCTGGCCTGGTATCAGCAGAAACC
CGGCCAGAGCCCTAAGCTGCTGATCTATTGGGCAAGTACCCGAGAGTCAG
GAGTGCCAGACAGATTCTCCGGGTCTGGAAGTGGCACAGACTTCACCCTG
ACAATTAGCTCCGTGCAGGCCGAGGACGTGGCTGTCTACTATTGCCAGCA
GTACTATAGCTACCGAACTTTCGGCGGGGGAACCAAACTGGAAATCAAGG
GAGGAGGAGGCAGTGGCGGAGGAGGGTCAGGAGGAGGAGGAAGCCAGGTG
CAGCTGCAGCAGTCCGGACCAGAGGTGGTCAAACCCGGCGCTAGCGTCAA
AATGTCCTGTAAGGCATCTGGCTACACTTTCACCTCTTATGTGATTCACT
GGGTCAGACAGAAGCCTGGGCAGGGACTGGACTGGATCGGGTACATTAAC
CCATATAATGATGGAACTGACTACGATGAAAAGTTTAAAGGCAAGGCCAC
ACTGACTTCCGACACCTCAACAAGCACTGCTTATATGGAGCTGTCTAGTC
TGAGGTCTGAAGACACAGCAGTGTACTATTGCGCCCGCGAGAAGGATAAC
TACGCCACTGGCGCTTGGTTTGCATATTGGGGCCAGGGGACCCTGGTGAC
AGTCTCATCCGCGGCCGCATTCGTGCCGGTCTTCCTGCCAGCGAAGCCCA
CCACGACGCCAGCGCCGCGACCACCAACACCGGCGCCCACCATCGCGTCG
CAGCCCCTGTCCCTGCGCCCAGAGGCGTGCCGGCCAGCGGCGGGGGGCGC
AGTGCACACGAGGGGGCTGGACTTCGCCTGTGATATCTACATCTGGGCGC
CCTTGGCCGGGACTTGTGGGGTCCTTCTCCTGTCACTGGTTATCACCCTT
TACTGCAACCACAGGAACAGGAGTAAGAGGAGCAGGCTCCTGCACAGTGA
CTACATGAACATGACTCCCCGCCGCCCCGGGCCCACCCGCAAGCATTACC
AGCCCTATGCCCCACCACGCGACTTCGCAGCCTATCGCTCCCGTTTCTCT
GTTGTTAAACGGGGCAGAAAGAAGCTCCTGTATATATTCAAACAACCATT
TATGAGACCAGTACAAACTACTCAAGAGGAAGATGGCTGTAGCTGCCGAT
TTCCAGAAGAAGAAGAAGGAGGATGTGAACTGAGAGTGAAGTTCAGCAGG
AGCGCAGACGCCCCCGCGTACCAGCAGGGCCAGAACCAGCTCTATAACGA
GCTCAATCTAGGACGAAGAGAGGAGTACGATGTTTTGGACAAGAGACGTG
GCCGGGACCCTGAGATGGGGGGAAAGCCGAGAAGGAAGAACCCTCAGGAA
GGCCTGTACAATGAACTGCAGAAAGATAAGATGGCGGAGGCCTACAGTGA
GATTGGGATGAAAGGCGAGCGCCGGAGGGGCAAGGGGCACGATGGCCTTT
ACCAGGGTCTCAGTACAGCCACCAAGGACACCTACGACGCCCTTCACATG
CAGGCCCTGCCCCCTCGCGGATCCGGAGCCACGAACTTCTCTCTGTTAAA
GCAAGCAGGAGACGTGGAAGAAAACCCCGGTCCTATGCTTCTCCTGGTGA
CAAGCCTTCTGCTCTGTGAGTTACCACACCCAGCATTCCTCCTGATCCCA
CGCAAAGTGTGTAACGGAATAGGTATTGGTGAATTTAAAGACTCACTCTC
CATAAATGCTACGAATATTAAACACTTCAAAAACTGCACCTCCATCAGTG
GCGATCTCCACATCCTGCCGGTGGCATTTAGGGGTGACTCCTTCACACAT
ACTCCTCCTCTGGATCCACAGGAACTGGATATTCTGAAAACCGTAAAGGA
AATCACAGGGTTTTTGCTGATTCAGGCTTGGCCTGAAAACAGGACGGACC
TCCATGCCTTTGAGAACCTAGAAATCATACGCGGCAGGACCAAGCAACAT
GGTCAGTTTTCTCTTGCAGTCGTCAGCCTGAACATAACATCCTTGGGATT
ACGCTCCCTCAAGGAGATAAGTGATGGAGATGTGATAATTTCAGGAAACA
AAAATTTGTGCTATGCAAATACAATAAACTGGAAAAAACTGTTTGGGACC
TCCGGTCAGAAAACCAAAATTATAAGCAACAGAGGTGAAAACAGCTGCAA
GGCCACAGGCCAGGTCTGCCATGCCTTGTGCTCCCCCGAGGGCTGCTGGG
GCCCGGAACCCAGGGACTGCGTCTCTTGCCGGAATGTCAGCCGAGGCAGG
GAATGCGTGGACAAGTGCAACCTTCTGGAGGGTGAGCCAAGGGAGTTTGT
GGAGAACTCTGAGTGCATACAGTGCCACCCAGAGTGCCTGCCTCAGGCCA
TGAACATCACCTGCACAGGACGGGGACCAGACAACTGTATCCAGTGTGCC
CACTACATTGACGGCCCCCACTGCGTCAAGACCTGCCCGGCAGGAGTCAT
GGGAGAAAACAACACCCTGGTCTGGAAGTACGCAGACGCCGGCCATGTGT
GCCACCTGTGCCATCCAAACTGCACCTACGGATGCACTGGGCCAGGTCTT
GAAGGCTGTCCAACGAATGGGCCTAAGATCCCGTCCATCGCCACTGGGAT
GGTGGGGGCCCTCCTCTTGCTGCTGGTGGTGGCCCTGGGGATCGGCCTCT
TCATGGGATCTGGAGCCACGAACTTCTCTCTGTTAAAGCAAGCAGGAGAC
GTGGAAGAAAACCCCGGTCCTATGCTCGAGGGAGTGCAGGTGGAAACCAT
CTCCCCAGGAGACGGGCGCACCTTCCCCAAGCGCGGCCAGACCTGCGTGG
TGCACTACACCGGGATGCTTGAAGATGGAAAGAAAGTTGATTCCTCCCGG
GACAGAAACAAGCCCTTTAAGTTTATGCTAGGCAAGCAGGAGGTGATCCG
AGGCTGGGAAGAAGGGGTTGCCCAGATGAGTGTGGGTCAGAGAGCCAAAC
TGACTATATCTCCAGATTATGCCTATGGTGCCACTGGGCACCCAGGCATC
ATCCCACCACATGCCACTCTCGTCTTCGATGTGGAGCTTCTAAAACTGGA
ATCTGGCGGTGGATCCGGAGTCGACGGATTTGGTGATGTCGGTGCTCTTG
AGAGTTTGAGGGGAAATGCAGATTTGGCTTACATCCTGAGCATGGAGCCC
TGTGGCCACTGCCTCATTATCAACAATGTGAACTTCTGCCGTGAGTCCGG
GCTCCGCACCCGCACTGGCTCCAACATCGACTGTGAGAAGTTGCGGCGTC
GCTTCTCCTCGCTGCATTTCATGGTGGAGGTGAAGGGCGACCTGACTGCC
AAGAAAATGGTGCTGGCTTTGCTGGAGCTGGCGCAGCAGGACCACGGTGC
TCTGGACTGCTGCGTGGTGGTCATTCTCTCTCACGGCTGTCAGGCCAGCC
ACCTGCAGTTCCCAGGGGCTGTCTACGGCACAGATGGATGCCCTGTGTCG
GTCGAGAAGATTGTGAACATCTTCAATGGGACCAGCTGCCCCAGCCTGGG
AGGGAAGCCCAAGCTCTTTTTCATCCAGGCCTGTGGTGGGGAGCAGAAAG
ACCATGGGTTTGAGGTGGCCTCCACTTCCCCTGAAGACGAGTCCCCTGGC
AGTAACCCCGAGCCAGATGCCACCCCGTTCCAGGAAGGTTTGAGGACCTT
CGACCAGCTGGACGCCATATCTAGTTTGCCCACACCCAGTGACATCTTTG
TGTCCTACTCTACTTTCCCAGGTTTTGTTTCCTGGAGGGACCCCAAGAGT
GGCTCCTGGTACGTTGAGACCCTGGACGACATCTTTGAGCAGTGGGCTCA
CTCTGAAGACCTGCAGTCCCTCCTGCTTAGGGTCGCTAATGCTGTTTCGG
TGAAAGGGATTTATAAACAGATGCCTGGTTGCTTTAATTTCCTCCGGAAA
AAACTTTTCTTTAAAACATCAGTCGACTATCCGTACGACGTACCAGACTA
CGCACTCGACTAA

Preferably, therefore, the construct comprises a nucleotide sequence substantially as set out in SEQ ID No: 30, or a fragment or variant thereof.

Hence, a second preferred embodiment of the nucleic acid construct (known as “CARTVb7.1”) has an amino acid sequence, referred to herein as SEQ ID No: 31, as follows:

[SEQ ID No: 31]
MALPVTALLLPLALLLHAARPDIQMTQSPSSLSASLGGKVTLTCKASQDI
NKYIAWYQHKPGKGPRLLIHYTSTLQPGIPSRFSGSGSGRDYSFSISNLE
PEDVATYYCLQYDNLRTFGGGTKLEIKRTDGGGGSGGGGSGGGGSQVQLQ
QPGAELVKPGASVKMSCKASGYTFTRYWITWVKQRPGQGLEWIGDIYPGS
GFTKYNEKFKSKATLTVDTSSSTAYMQLSSLTSEDSAVYYCAREGGNYWY
FDVWGTGTTVTVSSAAAAAFVPVFLPAKPTTTPAPRPPTPAPTIASQPLS
LRPEACRPAAGGAVHTRGLDFACDIYIWAPLAGTCGVLLLSLVITLYCNH
RNRSKRSRLLHSDYMNMTPRRPGPTRKHYQPYAPPRDFAAYRSRFSVVKR
GRKKLLYIFKQPFMRPVQTTQEEDGCSCRFPEEEEGGCELRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PRGSGATNFSLLKQAGDVEENPGPMLLLVTSLLLCELPHPAFLLIPRKVC
NGIGIGEFKDSLSINATNIKHFKNCTSISGDLHILPVAFRGDSFTHTPPL
DPQELDILKTVKEITGELLIQAWPENRTDLHAFENLEIIRGRTKQHGQFS
LAVVSLNITSLGLRSLKEISDGDVIISGNKNLCYANTINWKKLFGTSGQK
TKIISNRGENSCKATGQVCHALCSPEGCWGPEPRDCVSCRNVSRGRECVD
KCNLLEGEPREFVENSECIQCHPECLPQAMNITCTGRGPDNCIQCAHYID
GPHCVKTCPAGVMGENNTLVWKYADAGHVCHLCHPNCTYGCTGPGLEGCP
TNGPKIPSIATGMVGALLLLLVVALGIGLFMGSGATNFSLLKQAGDVEEN
PGPMLEGVQVETISPGDGRTFPKRGQTCVVHYTGMLEDGKKVDSSRDRNK
PFKFMLGKQEVIRGWEEGVAQMSVGQRAKLTISPDYAYGATGHPGIIPPH
ATLVFDVELLKLESGGGSGVDGFGDVGALESLRGNADLAYILSMEPCGHC
LIINNVNFCRESGLRTRTGSNIDCEKLRRRFSSLHFMVEVKGDLTAKKMV
LALLELAQQDHGALDCCVVVILSHGCQASHLQFPGAVYGTDGCPVSVEKI
VNIFNGTSCPSLGGKPKLFFIQACGGEQKDHGFEVASTSPEDESPGSNPE
PDATPFQEGLRTFDQLDAISSLPTPSDIFVSYSTFPGFVSWRDPKSGSWY
VETLDDIFEQWAHSEDLQSLLLRVANAVSVKGIYKQMPGCFNFLRKKLFF
KTSVDYPYDVPDYALD*

Preferably, therefore, the construct comprises a nucleotide sequence encoding an amino acid sequence substantially as set out in SEQ ID No: 31, or a fragment or variant thereof.

Preferably, the embodiment of the nucleic acid construct (known as “CARTVb7.1”) has a nucleotide sequence, referred to herein as SEQ ID No: 32, as follows:

[SEQ ID No: 32]
ATGGCTCTGCCTGTTACAGCTCTGCTGCTGCCTCTGGCTCTGCTTCTGCA
TGCCGCCAGACCTGACATCCAGATGACACAGAGCCCTAGCAGCCTGTCTG
CCTCTCTCGGCGGAAAAGTGACCCTGACATGCAAGGCCAGCCAGGACATC
AACAAGTATATCGCCTGGTATCAGCACAAGCCCGGCAAGGGACCTAGACT
GCTGATCCACTACACCAGCACACTGCAGCCTGGCATCCCCAGCAGATTTT
CTGGCAGCGGCTCCGGCAGAGACTACAGCTTCAGCATCAGCAACCTGGAA
CCTGAGGACGTGGCCACCTACTACTGCCTGCAGTACGACAACCTGCGGAC
CTTTGGCGGCGGAACAAAGCTGGAAATCAAGCGGACAGATGGCGGAGGCG
GATCAGGCGGCGGAGGAAGCGGTGGCGGAGGATCTCAAGTTCAGCTGCAA
CAGCCTGGCGCCGAGCTTGTGAAACCTGGCGCCTCTGTGAAGATGAGCTG
CAAGGCCTCCGGCTACACCTTCACCAGATACTGGATCACCTGGGTCAAGC
AGAGGCCTGGACAGGGACTCGAGTGGATCGGCGATATCTATCCTGGCTCC
GGCTTCACCAAGTACAACGAGAAGTTCAAGAGCAAGGCCACACTGACCGT
GGACACCAGCAGCAGCACAGCCTACATGCAGCTGTCTAGCCTGACCAGCG
AGGACAGCGCCGTGTACTACTGTGCTAGAGAAGGCGGCAACTACTGGTAC
TTCGACGTGTGGGGCACCGGCACCACAGTGACAGTTAGTTCTGCGGCCGC
GGCCGCATTCGTGCCGGTCTTCCTGCCAGCGAAGCCCACCACGACGCCAG
CGCCGCGACCACCAACACCGGCGCCCACCATCGCGTCGCAGCCCCTGTCC
CTGCGCCCAGAGGCGTGCCGGCCAGCGGCGGGGGGCGCAGTGCACACGAG
GGGGCTGGACTTCGCCTGTGATATCTACATCTGGGCGCCCTTGGCCGGGA
CTTGTGGGGTCCTTCTCCTGTCACTGGTTATCACCCTTTACTGCAACCAC
AGGAACAGGAGTAAGAGGAGCAGGCTCCTGCACAGTGACTACATGAACAT
GACTCCCCGCCGCCCCGGGCCCACCCGCAAGCATTACCAGCCCTATGCCC
CACCACGCGACTTCGCAGCCTATCGCTCCCGTTTCTCTGTTGTTAAACGG
GGCAGAAAGAAGCTCCTGTATATATTCAAACAACCATTTATGAGACCAGT
ACAAACTACTCAAGAGGAAGATGGCTGTAGCTGCCGATTTCCAGAAGAAG
AAGAAGGAGGATGTGAACTGAGAGTGAAGTTCAGCAGGAGCGCAGACGCC
CCCGCGTACCAGCAGGGCCAGAACCAGCTCTATAACGAGCTCAATCTAGG
ACGAAGAGAGGAGTACGATGTTTTGGACAAGAGACGTGGCCGGGACCCTG
AGATGGGGGGAAAGCCGAGAAGGAAGAACCCTCAGGAAGGCCTGTACAAT
GAACTGCAGAAAGATAAGATGGCGGAGGCCTACAGTGAGATTGGGATGAA
AGGCGAGCGCCGGAGGGGCAAGGGGCACGATGGCCTTTACCAGGGTCTCA
GTACAGCCACCAAGGACACCTACGACGCCCTTCACATGCAGGCCCTGCCC
CCTCGCGGATCCGGAGCCACGAACTTCTCTCTGTTAAAGCAAGCAGGAGA
CGTGGAAGAAAACCCCGGTCCTATGCTTCTCCTGGTGACAAGCCTTCTGC
TCTGTGAGTTACCACACCCAGCATTCCTCCTGATCCCACGCAAAGTGTGT
AACGGAATAGGTATTGGTGAATTTAAAGACTCACTCTCCATAAATGCTAC
GAATATTAAACACTTCAAAAACTGCACCTCCATCAGTGGCGATCTCCACA
TCCTGCCGGTGGCATTTAGGGGTGACTCCTTCACACATACTCCTCCTCTG
GATCCACAGGAACTGGATATTCTGAAAACCGTAAAGGAAATCACAGGGTT
TTTGCTGATTCAGGCTTGGCCTGAAAACAGGACGGACCTCCATGCCTTTG
AGAACCTAGAAATCATACGCGGCAGGACCAAGCAACATGGTCAGTTTTCT
CTTGCAGTCGTCAGCCTGAACATAACATCCTTGGGATTACGCTCCCTCAA
GGAGATAAGTGATGGAGATGTGATAATTTCAGGAAACAAAAATTTGTGCT
ATGCAAATACAATAAACTGGAAAAAACTGTTTGGGACCTCCGGTCAGAAA
ACCAAAATTATAAGCAACAGAGGTGAAAACAGCTGCAAGGCCACAGGCCA
GGTCTGCCATGCCTTGTGCTCCCCCGAGGGCTGCTGGGGCCCGGAACCCA
GGGACTGCGTCTCTTGCCGGAATGTCAGCCGAGGCAGGGAATGCGTGGAC
AAGTGCAACCTTCTGGAGGGTGAGCCAAGGGAGTTTGTGGAGAACTCTGA
GTGCATACAGTGCCACCCAGAGTGCCTGCCTCAGGCCATGAACATCACCT
GCACAGGACGGGGACCAGACAACTGTATCCAGTGTGCCCACTACATTGAC
GGCCCCCACTGCGTCAAGACCTGCCCGGCAGGAGTCATGGGAGAAAACAA
CACCCTGGTCTGGAAGTACGCAGACGCCGGCCATGTGTGCCACCTGTGCC
ATCCAAACTGCACCTACGGATGCACTGGGCCAGGTCTTGAAGGCTGTCCA
ACGAATGGGCCTAAGATCCCGTCCATCGCCACTGGGATGGTGGGGGCCCT
CCTCTTGCTGCTGGTGGTGGCCCTGGGGATCGGCCTCTTCATGGGATCTG
GAGCCACGAACTTCTCTCTGTTAAAGCAAGCAGGAGACGTGGAAGAAAAC
CCCGGTCCTATGCTCGAGGGAGTGCAGGTGGAAACCATCTCCCCAGGAGA
CGGGCGCACCTTCCCCAAGCGCGGCCAGACCTGCGTGGTGCACTACACCG
GGATGCTTGAAGATGGAAAGAAAGTTGATTCCTCCCGGGACAGAAACAAG
CCCTTTAAGTTTATGCTAGGCAAGCAGGAGGTGATCCGAGGCTGGGAAGA
AGGGGTTGCCCAGATGAGTGTGGGTCAGAGAGCCAAACTGACTATATCTC
CAGATTATGCCTATGGTGCCACTGGGCACCCAGGCATCATCCCACCACAT
GCCACTCTCGTCTTCGATGTGGAGCTTCTAAAACTGGAATCTGGCGGTGG
ATCCGGAGTCGACGGATTTGGTGATGTCGGTGCTCTTGAGAGTTTGAGGG
GAAATGCAGATTTGGCTTACATCCTGAGCATGGAGCCCTGTGGCCACTGC
CTCATTATCAACAATGTGAACTTCTGCCGTGAGTCCGGGCTCCGCACCCG
CACTGGCTCCAACATCGACTGTGAGAAGTTGCGGCGTCGCTTCTCCTCGC
TGCATTTCATGGTGGAGGTGAAGGGCGACCTGACTGCCAAGAAAATGGTG
CTGGCTTTGCTGGAGCTGGCGCAGCAGGACCACGGTGCTCTGGACTGCTG
CGTGGTGGTCATTCTCTCTCACGGCTGTCAGGCCAGCCACCTGCAGTTCC
CAGGGGCTGTCTACGGCACAGATGGATGCCCTGTGTCGGTCGAGAAGATT
GTGAACATCTTCAATGGGACCAGCTGCCCCAGCCTGGGAGGGAAGCCCAA
GCTCTTTTTCATCCAGGCCTGTGGTGGGGAGCAGAAAGACCATGGGTTTG
AGGTGGCCTCCACTTCCCCTGAAGACGAGTCCCCTGGCAGTAACCCCGAG
CCAGATGCCACCCCGTTCCAGGAAGGTTTGAGGACCTTCGACCAGCTGGA
CGCCATATCTAGTTTGCCCACACCCAGTGACATCTTTGTGTCCTACTCTA
CTTTCCCAGGTTTTGTTTCCTGGAGGGACCCCAAGAGTGGCTCCTGGTAC
GTTGAGACCCTGGACGACATCTTTGAGCAGTGGGCTCACTCTGAAGACCT
GCAGTCCCTCCTGCTTAGGGTCGCTAATGCTGTTTCGGTGAAAGGGATTT
ATAAACAGATGCCTGGTTGCTTTAATTTCCTCCGGAAAAAACTTTTCTTT
AAAACATCAGTCGACTATCCGTACGACGTACCAGACTACGCACTCGACTA
A

Preferably, therefore, the construct comprises a nucleotide sequence substantially as set out in SEQ ID No: 32, or a fragment or variant thereof.

Accordingly, it will be appreciated that the isolated MAIT cell obtained using the method of either the second or third aspect, may be activated, and is ultimately transduced with a nucleic acid construct according to the fifth aspect, which encodes the CAR, to thereby produce the CAR-MAIT cell of the first aspect or the fourth aspect.

In a sixth aspect, there is provided an expression vector encoding the nucleic acid construct of the fifth aspect.

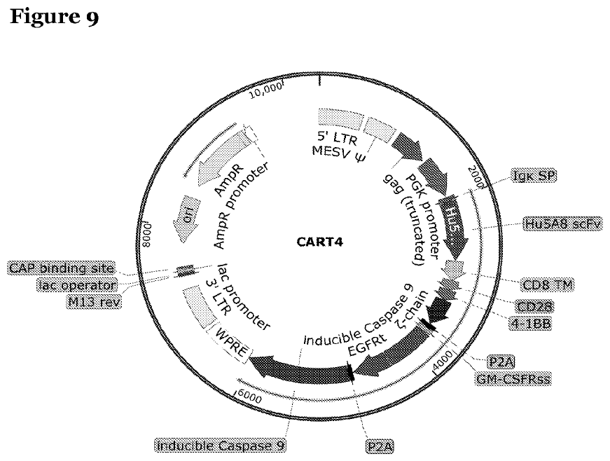

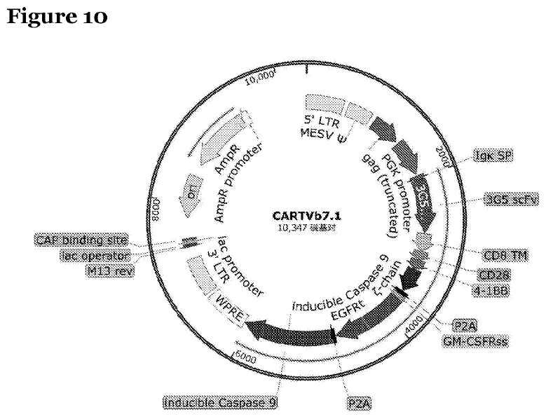

Preferably, the vector is recombinant. Preferably, the vector is a viral vector, more preferably a retroviral vector. Maps showing the features of two preferred embodiments of the vector of the invention are shown in FIGS. 9 and 10.

In an embodiment (CART4: CAR4-tEGFR-iC9; see FIG. 9), the vector has a nucleic acid sequence referred to herein as SEQ ID No: 33, as follows:

[SEQ ID NO: 33]
TGAAAGACCCCACCTGTAGGTTTGGCAAGCTAGCTTAAGTAACGCCATTTTGCAAGGCATGGAAAATACATAACT
GAGAATAGAGAAGTTCAGATCAAGGTTAGGAACAGAGAGACAGCAGAATATGGGCCAAACAGGATATCTGTGGTA
AGCAGTTCCTGCCCCGGCTCAGGGCCAAGAACAGATGGTCCCCAGATGCGGTCCCGCCCTCAGCAGTTTCTAGAG
AACCATCAGATGTTTCCAGGGTGCCCCAAGGACCTGAAATGACCCTGTGCCTTATTTGAACTAACCAATCAGTTC
GCTTCTCGCTTCTGTTCGCGCGCTTCTGCTCCCCGAGCTCAATAAAAGAGCCCACAACCCCTCACTCGGCGCGCC
AGTCCTCCGATAGACTGCGTCGCCCGGGTACCCGTATTCCCAATAAAGCCTCTTGCTGTTTGCATCCGAATCGTG
GACTCGCTGATCCTTGGGAGGGTCTCCTCAGATTGATTGACTGCCCACCTCGGGGGTCTTTCATTTGGAGGTTCC
ACCGAGATTTGGAGACCCCTGCCCAGGGACCACCGACCCCCCCGCCGGGAGGTAAGCTGGCCAGCGGTCGTTTCG
TGTCTGTCTCTGTCTTTGTGCGTGTTTGTGCCGGCATCTAATGTTTGCGCCTGCGTCTGTACTAGTTAGCTAACT
AGCTCTGTATCTGGCGGACCCGTGGTGGAACTGACGAGTTCTGAACACCCGGCCGCAACCCTGGGAGACGTCCCA
GGGACTTTGGGGGCCGTTTTTGTGGCCCGACCTGAGGAAGGGAGTCGATGTGGAATCCGACCCCGTCAGGATATG
TGGTTCTGGTAGGAGACGAGAACCTAAAACAGTTCCCGCCTCCGTCTGAATTTTTGCTTTCGGTTTGGAACCGAA
GCCGCGCGTCTTGTCTGCTGCAGCGCTGCAGCATCGTTCTGTGTTGTCTCTGTCTGACTGTGTTTCTGTATTTGT
CTGAAAATTAGGGCCAGACTGTTACCACTCCCTTAAGTTTGACCTTAGGTCACTGGAAAGATGTCGAGCGGATCG
CTCACAACCAGTCGGTAGATGTCAAGAAGAGACGTTGGGTTACCTTCTGCTCTGCAGAATGGCCAACCTTTAACG
TCGGATGGCCGCGAGACGGCACCTTTAACCGAGACCTCATCACCCAGGTTAAGATCAAGGTCTTTTCACCTGGCC
CGCATGGACACCCAGACCAGGTCCCCTACATCGTGACCTGGGAAGCCTTGGCTTTTGACCCCCCTCCCTGGGTCA
AGCCCTTTGTACACCCTAAGCCTCCGCCTCCTCTTCCTCCATCCGCCCCGTCTCTCCCCCTTGAACCTCCTCGTT
CGACCCCGCCTCGATCCTCCCTTTATCCAGCCCTCACTCCTTCTCTAGGCGCCGGAATTAGATCTCTCGAGGTTA
ACGAATTCTACCGGGTAGGGGAGGCGCTTTTCCCAAGGCAGTCTGGAGCATGCGCTTTAGCAGCCCCGCTGGGCA
CTTGGCGCTACACAAGTGGCCTCTGGCCTCGCACACATTCCACATCCACCGGTAGGCGCCAACCGGCTCCGTTCT
TTGGTGGCCCCTTCGCGCCACCTTCTACTCCTCCCCTAGTCAGGAAGTTCCCCCCCGCCCCGCAGCTCGCGTCGT
GCAGGACGTGACAAATGGAAGTAGCACGTCTCACTAGTCTCGTGCAGATGGACAGCACCGCTGAGCAATGGAAGC
GGGTAGGCCTTTGGGGCAGCGGCCAATAGCAGCTTTGCTCCTTCGCTTTCTGGGCTCAGAGGCTGGGAAGGGGTG
GGTCCGGGGGCGGGCTCAGGGGGGGGCTCAGGGGCGGGGGGGGCGCCCGAAGGTCCTCCGGAGGCCCGGCATTCT
GCACGCTTCAAAAGCGCACGTCTGCCGCGCTGTTCTCCTCTTCCTCATTCTCCGGGCCTTTCGACCTGCAGCCCA
AGCCACCATGGAGACAGACACACTCCTGCTATGGGTGCTGCTGCTCTGGGTTCCAGGTTCCACAGGTGACGACAT
TGTGATGACTCAGAGCCCCGACAGCCTGGCCGTCTCACTGGGCGAAAGGGTGACCATGAATTGTAAATCTTCTCA
GAGCCTGCTGTACAGTACAAACCAGAAAAATTACCTGGCCTGGTATCAGCAGAAACCCGGCCAGAGCCCTAAGCT
GCTGATCTATTGGGCAAGTACCCGAGAGTCAGGAGTGCCAGACAGATTCTCCGGGTCTGGAAGTGGCACAGACTT
CACCCTGACAATTAGCTCCGTGCAGGCCGAGGACGTGGCTGTCTACTATTGCCAGCAGTACTATAGCTACCGAAC
TTTCGGCGGGGGAACCAAACTGGAAATCAAGGGAGGAGGAGGCAGTGGCGGAGGAGGGTCAGGAGGAGGAGGAAG
CCAGGTGCAGCTGCAGCAGTCCGGACCAGAGGTGGTCAAACCCGGCGCTAGCGTCAAAATGTCCTGTAAGGCATC
TGGCTACACTTTCACCTCTTATGTGATTCACTGGGTCAGACAGAAGCCTGGGCAGGGACTGGACTGGATCGGGTA
CATTAACCCATATAATGATGGAACTGACTACGATGAAAAGTTTAAAGGCAAGGCCACACTGACTTCCGACACCTC
AACAAGCACTGCTTATATGGAGCTGTCTAGTCTGAGGTCTGAAGACACAGCAGTGTACTATTGCGCCCGCGAGAA
GGATAACTACGCCACTGGCGCTTGGTTTGCATATTGGGGCCAGGGGACCCTGGTGACAGTCTCATCCGCGGCCGC
ATTCGTGCCGGTCTTCCTGCCAGCGAAGCCCACCACGACGCCAGCGCCGCGACCACCAACACCGGCGCCCACCAT
CGCGTCGCAGCCCCTGTCCCTGCGCCCAGAGGCGTGCCGGCCAGCGGCGGGGGGCGCAGTGCACACGAGGGGGCT
GGACTTCGCCTGTGATATCTACATCTGGGCGCCCTTGGCCGGGACTTGTGGGGTCCTTCTCCTGTCACTGGTTAT
CACCCTTTACTGCAACCACAGGAACAGGAGTAAGAGGAGCAGGCTCCTGCACAGTGACTACATGAACATGACTCC
CCGCCGCCCCGGGCCCACCCGCAAGCATTACCAGCCCTATGCCCCACCACGCGACTTCGCAGCCTATCGCTCCCG
TTTCTCTGTTGTTAAACGGGGCAGAAAGAAGCTCCTGTATATATTCAAACAACCATTTATGAGACCAGTACAAAC
TACTCAAGAGGAAGATGGCTGTAGCTGCCGATTTCCAGAAGAAGAAGAAGGAGGATGTGAACTGAGAGTGAAGTT
CAGCAGGAGCGCAGACGCCCCCGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGGAAAGCCGAGAAGGAAGAACCC
TCAGGAAGGCCTGTACAATGAACTGCAGAAAGATAAGATGGCGGAGGCCTACAGTGAGATTGGGATGAAAGGCGA
GCGCCGGAGGGGCAAGGGGCACGATGGCCTTTACCAGGGTCTCAGTACAGCCACCAAGGACACCTACGACGCCCT
TCACATGCAGGCCCTGCCCCCTCGCGGATCCGGAGCCACGAACTTCTCTCTGTTAAAGCAAGCAGGAGACGTGGA
AGAAAACCCCGGTCCTATGCTTCTCCTGGTGACAAGCCTTCTGCTCTGTGAGTTACCACACCCAGCATTCCTCCT
GATCCCACGCAAAGTGTGTAACGGAATAGGTATTGGTGAATTTAAAGACTCACTCTCCATAAATGCTACGAATAT
TAAACACTTCAAAAACTGCACCTCCATCAGTGGCGATCTCCACATCCTGCCGGTGGCATTTAGGGGTGACTCCTT
CACACATACTCCTCCTCTGGATCCACAGGAACTGGATATTCTGAAAACCGTAAAGGAAATCACAGGGTTTTTGCT
GATTCAGGCTTGGCCTGAAAACAGGACGGACCTCCATGCCTTTGAGAACCTAGAAATCATACGCGGCAGGACCAA
GCAACATGGTCAGTTTTCTCTTGCAGTCGTCAGCCTGAACATAACATCCTTGGGATTACGCTCCCTCAAGGAGAT
AAGTGATGGAGATGTGATAATTTCAGGAAACAAAAATTTGTGCTATGCAAATACAATAAACTGGAAAAAACTGTT
TGGGACCTCCGGTCAGAAAACCAAAATTATAAGCAACAGAGGTGAAAACAGCTGCAAGGCCACAGGCCAGGTCTG
CCATGCCTTGTGCTCCCCCGAGGGCTGCTGGGGCCCGGAACCCAGGGACTGCGTCTCTTGCCGGAATGTCAGCCG
AGGCAGGGAATGCGTGGACAAGTGCAACCTTCTGGAGGGTGAGCCAAGGGAGTTTGTGGAGAACTCTGAGTGCAT
ACAGTGCCACCCAGAGTGCCTGCCTCAGGCCATGAACATCACCTGCACAGGACGGGGACCAGACAACTGTATCCA
GTGTGCCCACTACATTGACGGCCCCCACTGCGTCAAGACCTGCCCGGCAGGAGTCATGGGAGAAAACAACACCCT
GGTCTGGAAGTACGCAGACGCCGGCCATGTGTGCCACCTGTGCCATCCAAACTGCACCTACGGATGCACTGGGCC
AGGTCTTGAAGGCTGTCCAACGAATGGGCCTAAGATCCCGTCCATCGCCACTGGGATGGTGGGGGCCCTCCTCTT
GCTGCTGGTGGTGGCCCTGGGGATCGGCCTCTTCATGGGATCTGGAGCCACGAACTTCTCTCTGTTAAAGCAAGC
AGGAGACGTGGAAGAAAACCCCGGTCCTATGCTCGAGGGAGTGCAGGTGGAAACCATCTCCCCAGGAGACGGGCG
CACCTTCCCCAAGCGCGGCCAGACCTGCGTGGTGCACTACACCGGGATGCTTGAAGATGGAAAGAAAGTTGATTC
CTCCCGGGACAGAAACAAGCCCTTTAAGTTTATGCTAGGCAAGCAGGAGGTGATCCGAGGCTGGGAAGAAGGGGT
TGCCCAGATGAGTGTGGGTCAGAGAGCCAAACTGACTATATCTCCAGATTATGCCTATGGTGCCACTGGGCACCC
AGGCATCATCCCACCACATGCCACTCTCGTCTTCGATGTGGAGCTTCTAAAACTGGAATCTGGCGGTGGATCCGG
AGTCGACGGATTTGGTGATGTCGGTGCTCTTGAGAGTTTGAGGGGAAATGCAGATTTGGCTTACATCCTGAGCAT
GGAGCCCTGTGGCCACTGCCTCATTATCAACAATGTGAACTTCTGCCGTGAGTCCGGGCTCCGCACCCGCACTGG
CTCCAACATCGACTGTGAGAAGTTGCGGCGTCGCTTCTCCTCGCTGCATTTCATGGTGGAGGTGAAGGGCGACCT
GACTGCCAAGAAAATGGTGCTGGCTTTGCTGGAGCTGGCGCAGCAGGACCACGGTGCTCTGGACTGCTGCGTGGT
GGTCATTCTCTCTCACGGCTGTCAGGCCAGCCACCTGCAGTTCCCAGGGGCTGTCTACGGCACAGATGGATGCCC
TGTGTCGGTCGAGAAGATTGTGAACATCTTCAATGGGACCAGCTGCCCCAGCCTGGGAGGGAAGCCCAAGCTCTT
TTTCATCCAGGCCTGTGGTGGGGAGCAGAAAGACCATGGGTTTGAGGTGGCCTCCACTTCCCCTGAAGACGAGTC
CCCTGGCAGTAACCCCGAGCCAGATGCCACCCCGTTCCAGGAAGGTTTGAGGACCTTCGACCAGCTGGACGCCAT
ATCTAGTTTGCCCACACCCAGTGACATCTTTGTGTCCTACTCTACTTTCCCAGGTTTTGTTTCCTGGAGGGACCC
CAAGAGTGGCTCCTGGTACGTTGAGACCCTGGACGACATCTTTGAGCAGTGGGCTCACTCTGAAGACCTGCAGTC
CCTCCTGCTTAGGGTCGCTAATGCTGTTTCGGTGAAAGGGATTTATAAACAGATGCCTGGTTGCTTTAATTTCCT
CCGGAAAAAACTTTTCTTTAAAACATCAGTCGACTATCCGTACGACGTACCAGACTACGCACTCGACTAAACAAT
CAACCTCTGGATTACAAAATTTGTGAAAGATTGACTGGTATTCTTAACTATGTTGCTCCTTTTACGCTATGTGGA
TACGCTGCTTTAATGCCTTTGTATCATGCTATTGCTTCCCGTATGGCTTTCATTTTCTCCTCCTTGTATAAATCC
TGGTTGCTGTCTCTTTATGAGGAGTTGTGGCCCGTTGTCAGGCAACGTGGCGTGGTGTGCACTGTGTTTGCTGAC
GCAACCCCCACTGGTTGGGGCATTGCCACCACCTGTCAGCTCCTTTCCGGGACTTTCGCTTTCCCCCTCCCTATT
GCCACGGCGGAACTCATCGCCGCCTGCCTTGCCCGCTGCTGGACAGGGGCTCGGCTGTTGGGCACTGACAATTCC
GTGGTGTTGTCGGGGAAATCATCGTCCTTTCCTTGGCTGCTCGCCTGTGTTGCCACCTGGATTCTGCGCGGGACG
TCCTTCTGCTACGTCCCTTCGGCCCTCAATCCAGCGGACCTTCCTTCCCGCGGCCTGCTGCCGGCTCTGCGGCCT
CTTCCGCGTCTTCGCCTTCGCCCTCAGACGAGTCGGATCTCCCTTTGGGCCGCCTCCCCGCCTATCGATAAAATA
AAAGATTTTATTTAGTCTCCAGAAAAAGGGGGGAATGAAAGACCCCACCTGTAGGTTTGGCAAGCTAGCTTAAGT
AACGCCATTTTGCAAGGCATGGAAAATACATAACTGAGAATAGAGAAGTTCAGATCAAGGTTAGGAACAGAGAGA
CAGCAGAATATGGGCCAAACAGGATATCTGTGGTAAGCAGTTCCTGCCCCGGCTCAGGGCCAAGAACAGATGGTC
CCCAGATGCGGTCCCGCCCTCAGCAGTTTCTAGAGAACCATCAGATGTTTCCAGGGTGCCCCAAGGACCTGAAAT
GACCCTGTGCCTTATTTGAACTAACCAATCAGTTCGCTTCTCGCTTCTGTTCGCGCGCTTCTGCTCCCCGAGCTC
AATAAAAGAGCCCACAACCCCTCACTCGGCGCGCCAGTCCTCCGATAGACTGCGTCGCCCGGGTACCCGTGTATC
CAATAAACCCTCTTGCAGTTGCATCCGACTTGTGGTCTCGCTGTTCCTTGGGAGGGTCTCCTCTGAGTGATTGAC
TACCCGTCAGCGGGGGTCTTTCATGGGTAACAGTTTCTTGAAGTTGGAGAACAACATTCTGAGGGTAGGAGTCGA
ATATTAAGTAATCCTGACTCAATTAGCCACTGTTTTGAATCCACATACTCCAATACTCCTGAAATAGTTCATTAT
GGACAGCGCAGAAGAGCTGGGGAGAATTAATTCGTAATCATGGTCATAGCTGTTTCCTGTGTGAAATTGTTATCC
GCTCACAATTCCACACAACATACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCTGTCGTGCCAGCTGCATTAATGAATCGG
CCAACGCGCGGGGAGAGGCGGTTTGCGTATTGGGCGCTCTTCCGCTTCCTCGCTCACTGACTCGCTGCGCTCGGT
CGTTCGGCTGCGGCGAGCGGTATCAGCTCACTCAAAGGCGGTAATACGGTTATCCACAGAATCAGGGGATAACGC
AGGAAAGAACATGTGAGCAAAAGGCCAGCAAAAGGCCAGGAACCGTAAAAAGGCCGCGTTGCTGGCGTTTTTCCA
TAGGCTCCGCCCCCCTGACGAGCATCACAAAAATCGACGCTCAAGTCAGAGGTGGCGAAACCCGACAGGACTATA
AAGATACCAGGCGTTTCCCCCTGGAAGCTCCCTCGTGCGCTCTCCTGTTCCGACCCTGCCGCTTACCGGATACCT
GTCCGCCTTTCTCCCTTCGGGAAGCGTGGCGCTTTCTCATAGCTCACGCTGTAGGTATCTCAGTTCGGTGTAGGT
CGTTCGCTCCAAGCTGGGCTGTGTGCACGAACCCCCCGTTCAGCCCGACCGCTGCGCCTTATCCGGTAACTATCG
TCTTGAGTCCAACCCGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGGACAGTATTTGGTAT
CTGCGCTCTGCTGAAGCCAGTTACCTTCGGAAAAAGAGTTGGTAGCTCTTGATCCGGCAAACAAACCACCGCTGG
TAGCGGTGGTTTTTTTGTTTGCAAGCAGCAGATTACGCGCAGAAAAAAAGGATCTCAAGAAGATCCTTTGATCTT
TTCTACGGGGTCTGACGCTCAGTGGAACGAAAACTCACGTTAAGGGATTTTGGTCATGAGATTATCAAAAAGGAT
CTTCACCTAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTGCCTGACT
CCCCGTCGTGTAGATAACTACGATACGGGAGGGCTTACCATCTGGCCCCAGTGCTGCAATGATACCGCGAGACCC
ACGCTCACCGGCTCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCCTTCGGTCCTCCGATCGT
TGTCAGAAGTAAGTTGGCCGCAGTGTTATCACTCATGGTTATGGCAGCACTGCATAATTCTCTTACTGTCATGCC
ATCCGTAAGATGCTTTTCTGTGACTGGTGAGTACTCAACCAAGTCATTCTGAGAATAGTGTATGCGGCGACCGAG
TTGCTCTTGCCCGGCGTCAATACGGGATAATACCGCGCCACATAGCAGAACTTTAAAAGTGCTCATCATTGGAAA
ACGTTCTTCGGGGCGAAAACTCTCAAGGATCTTACCGCTGTTGAGATCCAGTTCGATGTAACCCACTCGTGCACC
CAACTGATCTTCAGCATCTTTTACTTTCACCAGCGTTTCTGGGTGAGCAAAAACAGGAAGGCAAAATGCCGCAAA
AAAGGGAATAAGGGCGACACGGAAATGTTGAATACTCATACTCTTCCTTTTTCAATATTATTGAAGCATTTATCA
GGGTTATTGTCTCATGAGCGGATACATATTTGAATGTATTTAGAAAAATAAACAAATAGGGGTTCCGCGCACATT
TCCCCGAAAAGTGCCACCTGACGTCTAAGAAACCATTATTATCATGACATTAACCTATAAAAATAGGCGTATCAC
GAGGCCCTTTCGTCTCGCGCGTTTCGGTGATGACGGTGAAAACCTCTGACACATGCAGCTCCCGGAGACGGTCAC
AGCTTGTCTGTAAGCGGATGCCGGGAGCAGACAAGCCCGTCAGGGCGCGTCAGCGGGTGTTGGCGGGTGTCGGGG
CTGGCTTAACTATGCGGCATCAGAGCAGATTGTACTGAGAGTGCACCATATGCGGTGTGAAATACCGCACAGATG
CGTAAGGAGAAAATACCGCATCAGGCGCCATTCGCCATTCAGGCTGCGCAACTGTTGGGAAGGGCGATCGGTGCG
GGCCTCTTCGCTATTACGCCAGCTGGCGAAAGGGGGATGTGCTGCAAGGCGATTAAGTTGGGTAACGCCAGGGTT
TTCCCAGTCACGACGTTGTAAAACGACGGCGCAAGGAATGGTGCATGCAAGGAGATGGCGCCCAACAGTCCCCCG
GCCACGGGGCCTGCCACCATACCCACGCCGAAACAAGCGCTCATGAGCCCGAAGTGGCGAGCCCGATCTTCCCCA
TCGGTGATGTCGGCGATATAGGCGCCAGCAACCGCACCTGTGGCGCCGGTGATGCCGGCCACGATGCGTCCGGCG
TAGAGGCGATTAGTCCAATTTGTTAAAGACAGGATATCAGTGGTCCAGGCTCTAGTTTTGACTCAACAATATCAC
CAGCTGAAGCCTATAGAGTACGAGCCATAGATAAAATAAAAGATTTTATTTAGTCTCCAGAAAAAGGGGGGAA

Preferably, the vector comprises a nucleic acid sequence substantially as set out in SEQ ID No: 33, or a fragment or variant thereof.

In an embodiment (CARTVb7.1: CARTVb7.1-tEGFR-iC9; see FIG. 10), the vector has a nucleic acid sequence referred to herein as SEQ ID No: 36, as follows:

[SEQ ID NO: 36]
TGAAAGACCCCACCTGTAGGTTTGGCAAGCTAGCTTAAGTAACGCCATTTTGCAAGGCATGGAAAATACATAACTGAG
AATAGAGAAGTTCAGATCAAGGTTAGGAACAGAGAGACAGCAGAATATGGGCCAAACAGGATATCTGTGGTAAGCAGT
TCCTGCCCCGGCTCAGGGCCAAGAACAGATGGTCCCCAGATGCGGTCCCGCCCTCAGCAGTTTCTAGAGAACCATCAG
ATGTTTCCAGGGTGCCCCAAGGACCTGAAATGACCCTGTGCCTTATTTGAACTAACCAATCAGTTCGCTTCTCGCTTC
TGTTCGCGCGCTTCTGCTCCCCGAGCTCAATAAAAGAGCCCACAACCCCTCACTCGGCGCGCCAGTCCTCCGATAGAC
TGCGTCGCCCGGGTACCCGTATTCCCAATAAAGCCTCTTGCTGTTTGCATCCGAATCGTGGACTCGCTGATCCTTGGG
AGGGTCTCCTCAGATTGATTGACTGCCCACCTCGGGGGTCTTTCATTTGGAGGTTCCACCGAGATTTGGAGACCCCTG
CCCAGGGACCACCGACCCCCCCGCCGGGAGGTAAGCTGGCCAGCGGTCGTTTCGTGTCTGTCTCTGTCTTTGTGCGTG
TTTGTGCCGGCATCTAATGTTTGCGCCTGCGTCTGTACTAGTTAGCTAACTAGCTCTGTATCTGGCGGACCCGTGGTG
GAACTGACGAGTTCTGAACACCCGGCCGCAACCCTGGGAGACGTCCCAGGGACTTTGGGGGCCGTTTTTGTGGCCCGA
CCTGAGGAAGGGAGTCGATGTGGAATCCGACCCCGTCAGGATATGTGGTTCTGGTAGGAGACGAGAACCTAAAACAGT
TCCCGCCTCCGTCTGAATTTTTGCTTTCGGTTTGGAACCGAAGCCGCGCGTCTTGTCTGCTGCAGCGCTGCAGCATCG
TTCTGTGTTGTCTCTGTCTGACTGTGTTTCTGTATTTGTCTGAAAATTAGGGCCAGACTGTTACCACTCCCTTAAGTT
TGACCTTAGGTCACTGGAAAGATGTCGAGCGGATCGCTCACAACCAGTCGGTAGATGTCAAGAAGAGACGTTGGGTTA
CCTTCTGCTCTGCAGAATGGCCAACCTTTAACGTCGGATGGCCGCGAGACGGCACCTTTAACCGAGACCTCATCACCC
AGGTTAAGATCAAGGTCTTTTCACCTGGCCCGCATGGACACCCAGACCAGGTCCCCTACATCGTGACCTGGGAAGCCT
TGGCTTTTGACCCCCCTCCCTGGGTCAAGCCCTTTGTACACCCTAAGCCTCCGCCTCCTCTTCCTCCATCCGCCCCGT
CTCTCCCCCTTGAACCTCCTCGTTCGACCCCGCCTCGATCCTCCCTTTATCCAGCCCTCACTCCTTCTCTAGGCGCCG
GAATTAGATCTCTCGAGGTTAACGAATTCTACCGGGTAGGGGAGGCGCTTTTCCCAAGGCAGTCTGGAGCATGCGCTT
TAGCAGCCCCGCTGGGCACTTGGCGCTACACAAGTGGCCTCTGGCCTCGCACACATTCCACATCCACCGGTAGGCGCC
AACCGGCTCCGTTCTTTGGTGGCCCCTTCGCGCCACCTTCTACTCCTCCCCTAGTCAGGAAGTTCCCCCCCGCCCCGC
AGCTCGCGTCGTGCAGGACGTGACAAATGGAAGTAGCACGTCTCACTAGTCTCGTGCAGATGGACAGCACCGCTGAGC
AATGGAAGCGGGTAGGCCTTTGGGGCAGCGGCCAATAGCAGCTTTGCTCCTTCGCTTTCTGGGCTCAGAGGCTGGGAA
GGGGTGGGTCCGGGGGCGGGCTCAGGGGGGGGCTCAGGGGCGGGGGGGGCGCCCGAAGGTCCTCCGGAGGCCCGGCAT
TCTGCACGCTTCAAAAGCGCACGTCTGCCGCGCTGTTCTCCTCTTCCTCATTCTCCGGGCCTTTCGACCTGCAGCCCA
AGCCACCATGGCTCTGCCTGTTACAGCTCTGCTGCTGCCTCTGGCTCTGCTTCTGCATGCCGCCAGACCTGACATCCA
GATGACACAGAGCCCTAGCAGCCTGTCTGCCTCTCTCGGCGGAAAAGTGACCCTGACATGCAAGGCCAGCCAGGACAT
CAACAAGTATATCGCCTGGTATCAGCACAAGCCCGGCAAGGGACCTAGACTGCTGATCCACTACACCAGCACACTGCA
GCCTGGCATCCCCAGCAGATTTTCTGGCAGCGGCTCCGGCAGAGACTACAGCTTCAGCATCAGCAACCTGGAACCTGA
GGACGTGGCCACCTACTACTGCCTGCAGTACGACAACCTGCGGACCTTTGGCGGCGGAACAAAGCTGGAAATCAAGCG
GACAGATGGCGGAGGCGGATCAGGCGGCGGAGGAAGCGGTGGCGGAGGATCTCAAGTTCAGCTGCAACAGCCTGGCGC
CGAGCTTGTGAAACCTGGCGCCTCTGTGAAGATGAGCTGCAAGGCCTCCGGCTACACCTTCACCAGATACTGGATCAC
CTGGGTCAAGCAGAGGCCTGGACAGGGACTCGAGTGGATCGGCGATATCTATCCTGGCTCCGGCTTCACCAAGTACAA
CGAGAAGTTCAAGAGCAAGGCCACACTGACCGTGGACACCAGCAGCAGCACAGCCTACATGCAGCTGTCTAGCCTGAC
CAGCGAGGACAGCGCCGTGTACTACTGTGCTAGAGAAGGCGGCAACTACTGGTACTTCGACGTGTGGGGCACCGGCAC
CACAGTGACAGTTAGTTCTGCGGCCGCGGCCGCATTCGTGCCGGTCTTCCTGCCAGCGAAGCCCACCACGACGCCAGC
GCCGCGACCACCAACACCGGCGCCCACCATCGCGTCGCAGCCCCTGTCCCTGCGCCCAGAGGCGTGCCGGCCAGCGGC
GGGGGGCGCAGTGCACACGAGGGGGCTGGACTTCGCCTGTGATATCTACATCTGGGCGCCCTTGGCCGGGACTTGTGG
GGTCCTTCTCCTGTCACTGGTTATCACCCTTTACTGCAACCACAGGAACAGGAGTAAGAGGAGCAGGCTCCTGCACAG
TGACTACATGAACATGACTCCCCGCCGCCCCGGGCCCACCCGCAAGCATTACCAGCCCTATGCCCCACCACGCGACTT
CGCAGCCTATCGCTCCCGTTTCTCTGTTGTTAAACGGGGCAGAAAGAAGCTCCTGTATATATTCAAACAACCATTTAT
GAGACCAGTACAAACTACTCAAGAGGAAGATGGCTGTAGCTGCCGATTTCCAGAAGAAGAAGAAGGAGGATGTGAACT
GAGAGTGAAGTTCAGCAGGAGCGCAGACGCCCCCGCGTACCAGCAGGGCCAGAACCAGCTCTATAACGAGCTCAATCT
AGGACGAAGAGAGGAGTACGATGTTTTGGACAAGAGACGTGGCCGGGACCCTGAGATGGGGGGAAAGCCGAGAAGGAA
GAACCCTCAGGAAGGCCTGTACAATGAACTGCAGAAAGATAAGATGGCGGAGGCCTACAGTGAGATTGGGATGAAAGG
CGAGCGCCGGAGGGGCAAGGGGCACGATGGCCTTTACCAGGGTCTCAGTACAGCCACCAAGGACACCTACGACGCCCT
TCACATGCAGGCCCTGCCCCCTCGCGGATCCGGAGCCACGAACTTCTCTCTGTTAAAGCAAGCAGGAGACGTGGAAGA
AAACCCCGGTCCTATGCTTCTCCTGGTGACAAGCCTTCTGCTCTGTGAGTTACCACACCCAGCATTCCTCCTGATCCC
ACGCAAAGTGTGTAACGGAATAGGTATTGGTGAATTTAAAGACTCACTCTCCATAAATGCTACGAATATTAAACACTT
CAAAAACTGCACCTCCATCAGTGGCGATCTCCACATCCTGCCGGTGGCATTTAGGGGTGACTCCTTCACACATACTCC
TCCTCTGGATCCACAGGAACTGGATATTCTGAAAACCGTAAAGGAAATCACAGGGTTTTTGCTGATTCAGGCTTGGCC
TGAAAACAGGACGGACCTCCATGCCTTTGAGAACCTAGAAATCATACGCGGCAGGACCAAGCAACATGGTCAGTTTTC
TCTTGCAGTCGTCAGCCTGAACATAACATCCTTGGGATTACGCTCCCTCAAGGAGATAAGTGATGGAGATGTGATAAT
TTCAGGAAACAAAAATTTGTGCTATGCAAATACAATAAACTGGAAAAAACTGTTTGGGACCTCCGGTCAGAAAACCAA
AATTATAAGCAACAGAGGTGAAAACAGCTGCAAGGCCACAGGCCAGGTCTGCCATGCCTTGTGCTCCCCCGAGGGCTG
CTGGGGCCCGGAACCCAGGGACTGCGTCTCTTGCCGGAATGTCAGCCGAGGCAGGGAATGCGTGGACAAGTGCAACCT
TCTGGAGGGTGAGCCAAGGGAGTTTGTGGAGAACTCTGAGTGCATACAGTGCCACCCAGAGTGCCTGCCTCAGGCCAT
GAACATCACCTGCACAGGACGGGGACCAGACAACTGTATCCAGTGTGCCCACTACATTGACGGCCCCCACTGCGTCAA
GACCTGCCCGGCAGGAGTCATGGGAGAAAACAACACCCTGGTCTGGAAGTACGCAGACGCCGGCCATGTGTGCCACCT
GTGCCATCCAAACTGCACCTACGGATGCACTGGGCCAGGTCTTGAAGGCTGTCCAACGAATGGGCCTAAGATCCCGTC
CATCGCCACTGGGATGGTGGGGGCCCTCCTCTTGCTGCTGGTGGTGGCCCTGGGGATCGGCCTCTTCATGGGATCTGG
AGCCACGAACTTCTCTCTGTTAAAGCAAGCAGGAGACGTGGAAGAAAACCCCGGTCCTATGCTCGAGGGAGTGCAGGT
GGAAACCATCTCCCCAGGAGACGGGCGCACCTTCCCCAAGCGCGGCCAGACCTGCGTGGTGCACTACACCGGGATGCT
TGAAGATGGAAAGAAAGTTGATTCCTCCCGGGACAGAAACAAGCCCTTTAAGTTTATGCTAGGCAAGCAGGAGGTGAT
CCGAGGCTGGGAAGAAGGGGTTGCCCAGATGAGTGTGGGTCAGAGAGCCAAACTGACTATATCTCCAGATTATGCCTA
TGGTGCCACTGGGCACCCAGGCATCATCCCACCACATGCCACTCTCGTCTTCGATGTGGAGCTTCTAAAACTGGAATC
TGGCGGTGGATCCGGAGTCGACGGATTTGGTGATGTCGGTGCTCTTGAGAGTTTGAGGGGAAATGCAGATTTGGCTTA
CATCCTGAGCATGGAGCCCTGTGGCCACTGCCTCATTATCAACAATGTGAACTTCTGCCGTGAGTCCGGGCTCCGCAC
CCGCACTGGCTCCAACATCGACTGTGAGAAGTTGCGGCGTCGCTTCTCCTCGCTGCATTTCATGGTGGAGGTGAAGGG
CGACCTGACTGCCAAGAAAATGGTGCTGGCTTTGCTGGAGCTGGCGCAGCAGGACCACGGTGCTCTGGACTGCTGCGT
GGTGGTCATTCTCTCTCACGGCTGTCAGGCCAGCCACCTGCAGTTCCCAGGGGCTGTCTACGGCACAGATGGATGCCC
TGTGTCGGTCGAGAAGATTGTGAACATCTTCAATGGGACCAGCTGCCCCAGCCTGGGAGGGAAGCCCAAGCTCTTTTT
CATCCAGGCCTGTGGTGGGGAGCAGAAAGACCATGGGTTTGAGGTGGCCTCCACTTCCCCTGAAGACGAGTCCCCTGG
CAGTAACCCCGAGCCAGATGCCACCCCGTTCCAGGAAGGTTTGAGGACCTTCGACCAGCTGGACGCCATATCTAGTTT
GCCCACACCCAGTGACATCTTTGTGTCCTACTCTACTTTCCCAGGTTTTGTTTCCTGGAGGGACCCCAAGAGTGGCTC
CTGGTACGTTGAGACCCTGGACGACATCTTTGAGCAGTGGGCTCACTCTGAAGACCTGCAGTCCCTCCTGCTTAGGGT
CGCTAATGCTGTTTCGGTGAAAGGGATTTATAAACAGATGCCTGGTTGCTTTAATTTCCTCCGGAAAAAACTTTTCTT
TAAAACATCAGTCGACTATCCGTACGACGTACCAGACTACGCACTCGACTAAACAATCAACCTCTGGATTACAAAATT
TGTGAAAGATTGACTGGTATTCTTAACTATGTTGCTCCTTTTACGCTATGTGGATACGCTGCTTTAATGCCTTTGTAT
CATGCTATTGCTTCCCGTATGGCTTTCATTTTCTCCTCCTTGTATAAATCCTGGTTGCTGTCTCTTTATGAGGAGTTG
TGGCCCGTTGTCAGGCAACGTGGCGTGGTGTGCACTGTGTTTGCTGACGCAACCCCCACTGGTTGGGGCATTGCCACC
ACCTGTCAGCTCCTTTCCGGGACTTTCGCTTTCCCCCTCCCTATTGCCACGGCGGAACTCATCGCCGCCTGCCTTGCC
CGCTGCTGGACAGGGGCTCGGCTGTTGGGCACTGACAATTCCGTGGTGTTGTCGGGGAAATCATCGTCCTTTCCTTGG
CTGCTCGCCTGTGTTGCCACCTGGATTCTGCGCGGGACGTCCTTCTGCTACGTCCCTTCGGCCCTCAATCCAGCGGAC
CTTCCTTCCCGCGGCCTGCTGCCGGCTCTGCGGCCTCTTCCGCGTCTTCGCCTTCGCCCTCAGACGAGTCGGATCTCC
CTTTGGGCCGCCTCCCCGCCTATCGATAAAATAAAAGATTTTATTTAGTCTCCAGAAAAAGGGGGGAATGAAAGACCC
CACCTGTAGGTTTGGCAAGCTAGCTTAAGTAACGCCATTTTGCAAGGCATGGAAAATACATAACTGAGAATAGAGAAG
TTCAGATCAAGGTTAGGAACAGAGAGACAGCAGAATATGGGCCAAACAGGATATCTGTGGTAAGCAGTTCCTGCCCCG
GCTCAGGGCCAAGAACAGATGGTCCCCAGATGCGGTCCCGCCCTCAGCAGTTTCTAGAGAACCATCAGATGTTTCCAG
GGTGCCCCAAGGACCTGAAATGACCCTGTGCCTTATTTGAACTAACCAATCAGTTCGCTTCTCGCTTCTGTTCGCGCG
CTTCTGCTCCCCGAGCTCAATAAAAGAGCCCACAACCCCTCACTCGGCGCGCCAGTCCTCCGATAGACTGCGTCGCCC
GGGTACCCGTGTATCCAATAAACCCTCTTGCAGTTGCATCCGACTTGTGGTCTCGCTGTTCCTTGGGAGGGTCTCCTC
TGAGTGATTGACTACCCGTCAGCGGGGGTCTTTCATGGGTAACAGTTTCTTGAAGTTGGAGAACAACATTCTGAGGGT
AGGAGTCGAATATTAAGTAATCCTGACTCAATTAGCCACTGTTTTGAATCCACATACTCCAATACTCCTGAAATAGTT
CATTATGGACAGCGCAGAAGAGCTGGGGAGAATTAATTCGTAATCATGGTCATAGCTGTTTCCTGTGTGAAATTGTTA
TCCGCTCACAATTCCACACAACATACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGCTCGGTCGTTCG
GCTGCGGCGAGCGGTATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTTCCATAGGCTCCGCCC
CCCTGACGAGCATCACAAAAATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTGTCCGCCTTTCTCCCTTC
GGGAAGCGTGGCGCTTTCTCATAGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATGTAGGCGGTGCTACAGAGTTCTT
GAAGTGGTGGCCTAACTACGGCTACACTAGAAGGACAGTATTTGGTATCTGCGCTCTGCTGAAGCCAGTTACCTTCGG
AAAAAGAGTTGGTAGCTCTTGATCCGGCAAACAAACCACCGCTGGTAGCGGTGGTTTTTTTGTTTGCAAGCAGCAGAT
TACGCGCAGAAAAAAAGGATCTCAAGAAGATCCTTTGATCTTTTCTACGGGGTCTGACGCTCAGTGGAACGAAAACTC
ACGTTAAGGGATTTTGGTCATGAGATTATCAAAAAGGATCTTCACCTAGATCCTTTTAAATTAAAAATGAAGTTTTAA
ATCAATCTAAAGTATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGGCTTACCATCTGG
CCCCAGTGCTGCAATGATACCGCGAGACCCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTGGTGTCACGCTCGTCGTTTGGTAT
GGCTTCATTCAGCTCCGGTTCCCAACGATCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCACTCATGGTTATGGCAGCACTGCATAATTC
TCTTACTGTCATGCCATCCGTAAGATGCTTTTCTGTGACTGGTGAGTACTCAACCAAGTCATTCTGAGAATAGTGTAT
GCGGCGACCGAGTTGCTCTTGCCCGGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAACAGGAAGGCAAAATGCCGC
AAAAAAGGGAATAAGGGCGACACGGAAATGTTGAATACTCATACTCTTCCTTTTTCAATATTATTGAAGCATTTATCA
GGGTTATTGTCTCATGAGCGGATACATATTTGAATGTATTTAGAAAAATAAACAAATAGGGGTTCCGCGCACATTTCC
CCGAAAAGTGCCACCTGACGTCTAAGAAACCATTATTATCATGACATTAACCTATAAAAATAGGCGTATCACGAGGCC
CTTTCGTCTCGCGCGTTTCGGTGATGACGGTGAAAACCTCTGACACATGCAGCTCCCGGAGACGGTCACAGCTTGTCT
GTAAGCGGATGCCGGGAGCAGACAAGCCCGTCAGGGCGCGTCAGCGGGTGTTGGCGGGTGTCGGGGCTGGCTTAACTA
TGCGGCATCAGAGCAGATTGTACTGAGAGTGCACCATATGCGGTGTGAAATACCGCACAGATGCGTAAGGAGAAAATA
CCGCATCAGGCGCCATTCGCCATTCAGGCTGCGCAACTGTTGGGAAGGGCGATCGGTGCGGGCCTCTTCGCTATTACG
CCAGCTGGCGAAAGGGGGATGTGCTGCAAGGCGATTAAGTTGGGTAACGCCAGGGTTTTCCCAGTCACGACGTTGTAA
AACGACGGCGCAAGGAATGGTGCATGCAAGGAGATGGCGCCCAACAGTCCCCCGGCCACGGGGCCTGCCACCATACCC
ACGCCGAAACAAGCGCTCATGAGCCCGAAGTGGCGAGCCCGATCTTCCCCATCGGTGATGTCGGCGATATAGGCGCCA
GCAACCGCACCTGTGGCGCCGGTGATGCCGGCCACGATGCGTCCGGCGTAGAGGCGATTAGTCCAATTTGTTAAAGAC
AGGATATCAGTGGTCCAGGCTCTAGTTTTGACTCAACAATATCACCAGCTGAAGCCTATAGAGTACGAGCCATAGATA
AAATAAAAGATTTTATTTAGTCTCCAGAAAAAGGGGGGAA

Preferably, the vector comprises a nucleic acid sequence substantially as set out in SEQ ID No: 36, or a fragment or variant thereof.

Accordingly, it will be appreciated that the isolated MAIT cell obtained using the method of either the second or third aspect, may be activated, and is ultimately transduced with the expression vector of the according to the sixth aspect, which encodes the CAR, to thereby produce the CAR-MAIT cell of the first aspect or the fourth aspect.

In an eleventh aspect, there is provided a T-cell comprising the construct according to the fifth construct, or the vector according to the sixth aspect, optionally wherein the T-cell expresses an anti-CD4 chimeric antigen receptor (CAR) or anti-Vbeta CAR.

Preferably, the T-cell is a mucosal-associated invariant T (MAIT) cell.

In a seventh aspect, there is provided a pharmaceutical composition comprising a T-cell according to the eleventh aspect, preferably a MAIT cell according to the first aspect or fourth aspect, and a pharmaceutically acceptable excipient.

Preferably, the pharmaceutical composition comprises a plurality of the T cell or MAIT cell of the invention. For example, the composition may comprise at least 100, 1000, or 10,000 T cells or MAIT cells. Preferably, the composition comprises at least 100,000, or at least 1,000,000 or at least 10,000,000 T cells or MAIT cells.

In an eighth aspect, there is provided the T cell according to the eleventh aspect, or the MAIT cell according to the first aspect or fourth aspect, or the pharmaceutical composition of the seventh aspect, for use in therapy.

In a ninth aspect, there is provided the T cell according to the eleventh aspect, or the MAIT cell according to the first aspect or fourth aspect, or the pharmaceutical composition of the seventh aspect, for use in (i) immunotherapy; (ii) for treating, preventing or ameliorating cancer; (ii) for treating, preventing or ameliorating a microbial infection; or (iv) for treating, preventing or ameliorating an autoimmune disease.

In a tenth aspect, the invention provides a method of: (i) treating, preventing or ameliorating a disease in a subject with immunotherapy; (ii) treating, preventing or ameliorating cancer; (iii) for treating, preventing or ameliorating a microbial infection in a subject; or (iv) for treating, preventing or ameliorating an autoimmune disease in a subject, the method comprising administering, or having administered, to a patient in need of such treatment, a therapeutically effective amount of the T cell according to the eleventh aspect, or the MAIT cell according to the first aspect or fourth aspect, or the pharmaceutical composition of the seventh aspect.

Preferably, the T cell, MAIT cell or pharmaceutical composition is for use in treating, preventing or ameliorating a T-cell malignancy, which may be a solid tumour or a liquid tumour.

The T-cell malignancy may be a Peripheral T-cell lymphoma (PTCL) or a Cutaneous T-cell lymphoma (CTCL).

Peripheral T-cell lymphoma (PTCL) comprises a diverse group of uncommon and aggressive diseases in which the patient's T cells become cancerous. PTCLs are divided into three categories, i.e. nodal, extranodal and leukaemic, each of which are encompassed by the invention.

The PTCL may be a PTCL subtype selected from a group consisting of: Adult T-Cell Acute Lymphoblastic Lymphoma or Leukaemia (ATL); Enteropathy-Associated Lymphoma; Hepatosplenic Lymphoma; Subcutaneous Panniculitis-Like Lymphoma (SPTCL); Precursor T-Cell Acute Lymphoblastic Lymphoma or Leukaemia; and Angioimmunoblastic T-cell lymphoma (AITL).

Adult T-Cell Acute Lymphoblastic Lymphoma or Leukaemia (ATL) is more commonly found in Japan and the Caribbean than in the United States, and is associated with the human T-cell leukaemia virus-1 (HTLV-1). Enteropathy-Associated Lymphoma is associated with celiac disease, a chronic intestinal disorder caused by a hypersensitivity to gluten proteins found in wheat, rye and barley. Symptoms usually include stomach pain, weight loss, gastrointestinal bleeding or bowel perforation. Treatment for patients with enteropathy-associated T-cell lymphoma includes an anthracycline-based chemotherapy regimen, nutritional supplements and, if appropriate, a gluten-free diet. Hepatosplenic Lymphoma is an extremely rare and aggressive disease that starts in the liver or spleen and usually affects young adults in their 20s and 30s. Treatment for patients with hepatosplenic T-cell lymphoma includes anthracycline-based chemotherapy and, in some cases, stem cell transplantation.

Subcutaneous Panniculitis-Like Lymphoma (SPTCL) is the rarest and least well-defined of the T-cell lymphomas. This lymphoma occurs primarily in the subcutaneous fat tissue, where it causes nodules to form. Symptoms include fever, chills, weight loss and oral mucosal ulcers. SPTCL may be either rapidly aggressive or indolent (slow growing). Treatment includes combination anthracycline-based chemotherapy or localized radiation. Precursor T-Cell Acute Lymphoblastic Lymphoma or Leukaemia may be diagnosed as leukaemia or lymphoma or both. This cancer is found in both children and adults and is most commonly diagnosed in adolescent and adult males. Treatment for newly diagnosed patients with precursor T-cell acute lymphoblastic lymphoma or leukemia is aggressive chemotherapy and radiation. Nelarabine (Arranon®) is approved for the treatment of relapsed or refractory precursor T-cell acute lymphoblastic lymphoma or leukemia in adults and children.

Angioimmunoblastic T-cell lymphoma (AITL) exemplifies a neoplasm characterized by intense inflammatory and immune reactions, as evidenced by its clinical, pathologic, cellular, and biologic properties. Because tumour cells phenotypically resemble T follicular helper (Tfh) cells, they are considered to function similarly to some extent to nonneoplastic Tfh cells seen in reactive follicular hyperplasia. However, follicles are not hyperplastic but are rather depleted or destroyed in vast majority of AITL cases. AITL was recently reported to account for 36.1% of PTCL.

Cutaneous T-cell lymphoma (CTCL) constitute about 70-75% of the primary cutaneous lymphomas. The CTCL may be a CTCL subtype selected from a group consisting of: Mycosis fungoides (MF); Sezary syndrome (SS); and CD4+small medium pleomorphic T-cell lymphoproliferative disorder.

Mycosis fungoides (MF) is the most common subtype. Sezary syndrome (SS) is a more aggressive type of CTCL. Patients with SS have erythroderma (i.e. rash affecting >80% body surface area [BSA]), lymphadenopathy, and high numbers of circulating neoplastic CD4+ T cells in the peripheral blood.

In other embodiments, the T cell, MAIT cell or pharmaceutical composition may be used in treating, preventing or ameliorating a viral (e.g. HIV, HBV, HTLV, EBV, HPV), bacterial (e.g. TB), or fungal infection.

In other embodiments, the T cell, MAIT cell or pharmaceutical composition may be used in treating, preventing or ameliorating an autoimmune disease, for example systemic lupus erythematosus, rheumatoid arthritis, or myasthenia gravis.

Preferably, the method comprises triggering the sequence encoding the suicide protein. Accordingly, in embodiments in which the nucleic acid construct comprises a sequence encoding EGFR or tEGFR, the method preferably comprises administering, to the subject, an anti-EGFR antibody. For example, the anti-EGFR antibody may be the monoclonal antibody, Cetuximab. Administration of the antibody enables monitoring or depletion of the CAR-T cells in the subject.

In embodiments in which the nucleic acid construct comprises a sequence encoding iC9, the method may comprise administering, to the subject, a caspase-inducible drug (CID). For example, the CID may comprise Rimiducid. Administration of the CID enables conditional dimerization of the caspase, which triggers apoptosis of the CAR-T cells expressing the fusion protein, and resultant depletion of the CAR-T cells in the subject.

In a most preferred embodiment, the construct encodes two suicide proteins, including both iC9 and tEGFR, and so the use of an anti-EGFR antibody and a CID enables exquisite control of the life-span of the CAR-T or CAR-MAIT cells in the subject undergoing treatment.

It will be appreciated that the CAR-T cells or CAR-MAIT cells according to the invention (collectively referred to herein as “agents”) may be used in a monotherapy (e.g. the use of the T-cell or CAR-MAIT cell alone), for therapy, preferably in immunotherapy, for (i) treating, ameliorating or preventing cancer, or T-cell malignancies; or (ii) for treating, preventing or ameliorating a microbial infection, or (iii) an autoimmune disease. Alternatively, CAR-T or CAR-MAIT cells according to the invention may be used as an adjunct to, or in combination with, known immunotherapies or for treating microbial infections as well as cancers or autoimmune disease.

The agents according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising agents of the invention may be used in a number of ways. For instance, oral administration may be required, in which case the agents may be contained within a composition that may, for example, be ingested orally in the form of a tablet, capsule or liquid. Compositions comprising agents and medicaments of the invention may be administered by inhalation (e.g. intranasally). Compositions may also be formulated for topical use. For instance, creams or ointments may be applied to the skin.

Agents and medicaments according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with agents used according to the invention is required and which would normally require frequent administration (e.g. at least daily injection).

In a preferred embodiment, agents and medicaments according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the genetic construct or the vector (i.e. agent) that is required is determined by its biological activity and bioavailability, which in turn depends on the mode of administration, the physiochemical properties of the agent, and whether it is being used as a monotherapy or in a combined therapy. The frequency of administration will also be influenced by the half-life of the agent within the subject being treated. Optimal dosages to be administered may be determined by those skilled in the art, and will vary with the particular agent in use, the strength of the pharmaceutical composition, the mode of administration, and the advancement of the disease being treated, for example cancer, T-cell malignancies, microbial infection, or autoimmune disease. Additional factors depending on the particular subject being treated will result in a need to adjust dosages, including subject age, weight, gender, diet, and time of administration.

Generally, a daily dose of between 0.001 μg/kg of body weight and 10 mg/kg of body weight of agent according to the invention may be used for therapy, and in particular for treating, ameliorating, or preventing cancer, T-cell malignancies, microbial infection, or autoimmune disease, depending upon which agent. More preferably, the daily dose of agent is between 0.01 μg/kg of body weight and 1 mg/kg of body weight, more preferably between 0.1 μg/kg and 100 μg/kg body weight, and most preferably between approximately 0.1 μg/kg and 10 μg/kg body weight.

Alternatively, the dose administered to a subject may be between 0.5×10⁷ and 5×10¹² Transducing Units (TU)/Kg of body weight. More preferably, the dose administered to a subject may be between 0.5×10⁸ to 5×10¹¹ TU/Kg of body weight. Most preferably, the dose administered to a subject may be between 0.5×10⁹ to 5×10¹⁰ TU/Kg of body weight.

The agent may be administered before, during or after onset of the cancer, T-cell malignancy, microbial infection, or autoimmune disease. Daily doses may be given as a single administration (e.g. a single daily injection). Alternatively, the agent may require administration twice or more times during a day. As an example, agents may be administered as two (or more depending upon the severity of the disease being treated, for example cancer) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, the agent may require administration once a week for even once a month. Alternatively, a slow release device may be used to provide optimal doses of agents according to the invention to a patient without the need to administer repeated doses. Known procedures, such as those conventionally employed by the pharmaceutical industry (e.g. in vivo experimentation, clinical trials, etc.), may be used to form specific formulations of the agents according to the invention and precise therapeutic regimes (such as daily doses of the agents and the frequency of administration).

The pharmaceutical composition of the invention is preferably an immunotherapy treatment composition, or an autoimmune disease treatment composition, or anti-infection composition, or an anti-cancer composition, i.e. a pharmaceutical formulation used in the therapeutic amelioration, prevention or treatment of cancer in a subject.

The invention also provides in an eleventh aspect, a process for making the pharmaceutical composition according to the seventh aspect, the process comprising combining a therapeutically effective amount of the MAIT cell according to the first or fourth aspect and a pharmaceutically acceptable vehicle.

A “subject” may be a vertebrate, mammal, or domestic animal. Hence, medicaments according to the invention may be used to treat any mammal, for example livestock (e.g. a horse), pets, or may be used in other veterinary applications. Most preferably, the subject is a human being.

A “therapeutically effective amount” of the genetic construct or the vector is any amount which, when administered to a subject, is the amount of agent that is needed to treat the disease being treated, for example cancer, or produce the desired effect.

For example, the therapeutically effective amount of the genetic construct or the vector used may be from about 0.001 ng to about 1 mg, and preferably from about 0.01 ng to about 100 ng. It is preferred that the amount the genetic construct or the vector is an amount from about 0.1 ng to about 10 ng, and most preferably from about 0.5 ng to about 5 ng.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-36 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on: (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3×sodium chloride/sodium citrate (SSC) at approximately 45ºC followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in those of SEQ ID Nos: 1 to 36 that are amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

FIG. 1 shows the generation of third-generation CD4-targeting T cells according to an embodiment of the invention. A(1). The diagram represents the functional elements included in one embodiment of a CAR construct according to the invention (known as “CART4”). The scFv derived from monoclonal antibody Hu5A8 was fused with a CD8 transmembrane domain™, a CD28 endodomain, a 4-1BB endodomain and the CD3 ζ chain. The gene sequences of tEGFR (truncated epidermal growth factor receptor) and iC9 (inducible caspase-9) were tagged behind CAR via self-cleaving 2A linkers. A(2). The diagram represents the functional elements included in another embodiment of a CAR construct according to the invention (known as “CARTVb7.1”). The scFv derived from monoclonal antibody 3G5 was fused with a CD8 transmembrane domain (TM), a CD28 endodomain, a 4-1BB endodomain and the CD3ζ chain. The gene sequences of tEGFR (truncated epidermal growth factor receptor) and iC9 (inducible caspase-9) were tagged behind CAR via self-cleaving 2A linkers. B. Transduced T cells were stained by anti-mouse IgG F(ab)′₂ antibody and anti-EGFR antibody. Cells were propagated on CD3⁺single lymphocytes, and numbers indicate the percentage of CAR⁺/tEGFR⁺ cells. C. After retroviral transduction of CAR, primary T cells were sampled every day and stained for surface markers, including CD3 and tEGFR. The blue histogram was the result of non-transduced cells. The percentages of cells positive for CAR and marker are shown in the plots. D. Survival ratio was defined as the ratio of the EGFR-positive (A) or EGFR-high (B) percentage from untreated condition and chemical inducer of dimerization (CID, Rimiducid)-treated conditions 24 hours after the exposure to the indicated doses of CID. E. shows the mean fluorescent intensity of EGFR expression in survived cells. Data reflects typical results from three replicates from separate donors. Three replicates of each sample were performed. Data are represented as mean±SEM. Statistically significant difference was found between groups as determined by a two-tailed unpaired Student's t-test. **=p<0.01; *=p<0.05.

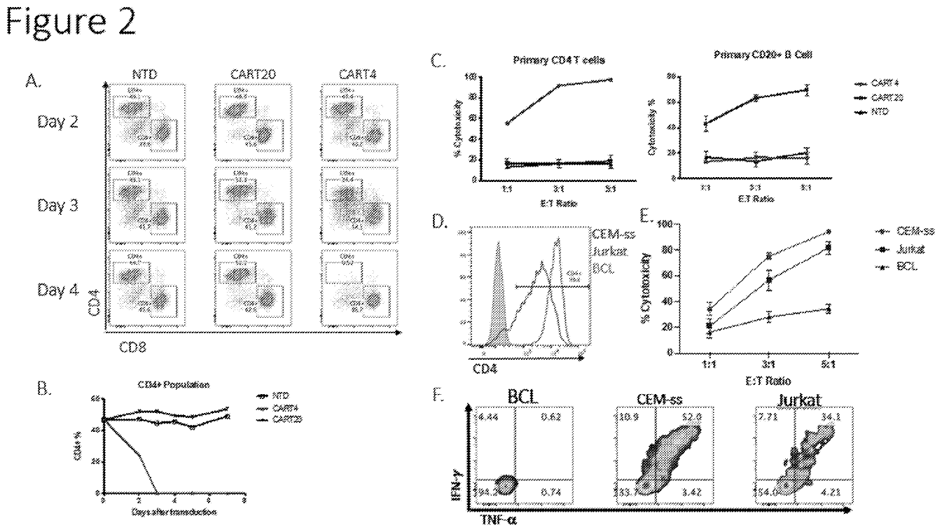

FIG. 2 shows the functional validation in vitro of an embodiment of CART4 T cells according to the invention. A. PBMCs were activated by Dynabeads Human T-Activator and IL7/IL15. The activated PBMCs contained two subsets of T cells, CD4⁺ and CD8⁺(left). Cells were either transduced by CART20 (middle) or CART4 (right) retroviral particles. From the third day after transduction, cells were stained by anti-CD4 and anti-CD8 antibodies and analysed by flow cytometry. The statistics of CD4⁺ratio were summarized in B. Data reflects typical results from five healthy individuals. C. Primary CD4⁺T cells (left) or CD20⁺B cells (right) were co-cultured with either autologous CART4 cells, CART20 cells, or non-transduced CD8⁺T cells for 4 hours. The absolute quantity of survived target cells was counted using Countbright beads by flow cytometry analysis. D. Two T cell lines CEM-ss cell, Jurkat cell and one B cell line were stained by the anti-CD4 antibody. The CD4 expression level was assessed by flow cytometry analysis. E. Representative result of CART4 cells killing T tumour cells. Three replicates of each sample were performed. Data reflects typical results from three independent experiments. F. Intracellular cytokine expression of CART4 cells co-cultured with different target cells. Three replicates of each sample were performed. Data reflects typical results from three independent experiments.

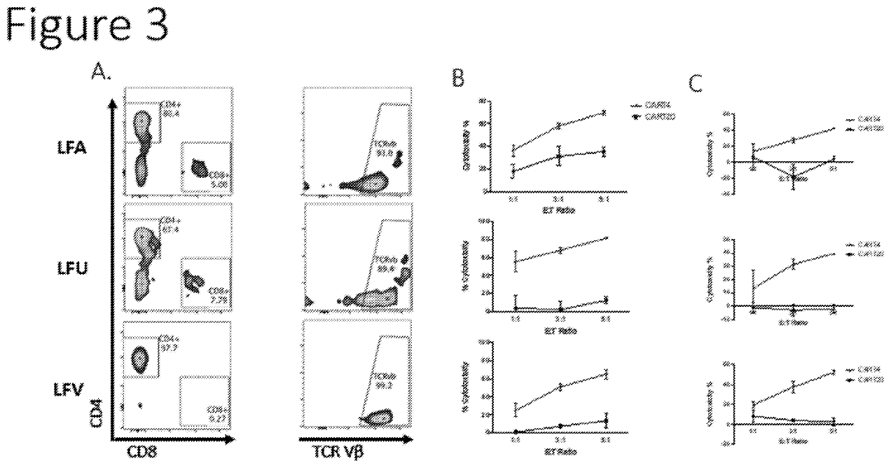

FIG. 3 shows CART4 cells specifically kill CD4+T tumor cells. PBMC vials from ATLL patients were revived from liquid nitrogen and rested in the incubator overnight before flow cytometry analysis and co-culture experiment. A. The PBMCs were stained by anti-CD4, CD8 and specific TCR VB. Flow cytometry was performed after two washes with PBS. Revived ATLL (B) or CTCL (C) PBMCs were co-cultured with allogenic CART4 or CART20 cells for four hours before flow cytometry analysis. Three replicates of each condition were performed. Data are represented as mean±SEM.

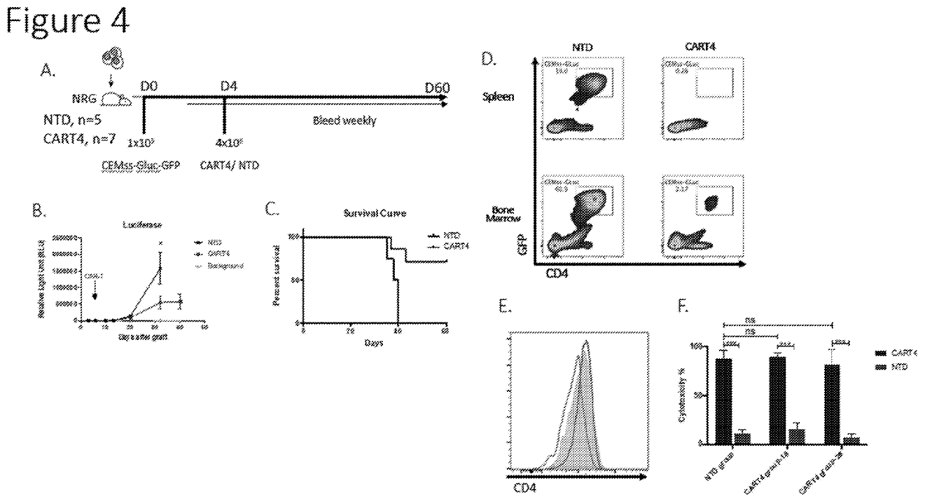

FIG. 4 shows that CART4 cells efficiently mediate antileukemic effects in vivo. A. NRG immunodeficient mice were injected with 1×10⁵ Gluc/GFP-transduced CEM-ss cells, followed by another infusion of 4×10⁶ T cells via the retro-orbital route. NTD n=5, CART4 n=7. B. 50 μl of peripheral blood of each mouse was bled and the plasma was used for the measurement of luciferase activity. Serial measurement of luciferase activity shown inhibition of CD4⁺leukaemia by CART4 T cells but not NTD CD8⁺T cells. C. Overall survival of mice treated with the indicated CART4 cells or the control NTD T cells by Kaplan-Meier survival analysis. D. At the endpoint, the mice were dissected. The spleens and bone marrows were ground and stained by anti-CD4 mAb and DAPI for detection of residual tumour cells. Tumour cells were identified as DAPI-CD4⁺GFP⁺. E. The CD4 expression level of residual tumour cells in spleens. Grey line-cultured CEMss cells, black line-CEMss cells from NTD control mice, red line-CEMss cells from CART4 treated mice. F. The splenic cells were co-cultured with CART4 cells or NTD T cells in 1:5 ratio for 4 hours, before being analysed by flow cytometry. Data are represented as mean±SEM. A two-tailed unpaired Student's t-test was used for significance analysis. *=p<0.05.

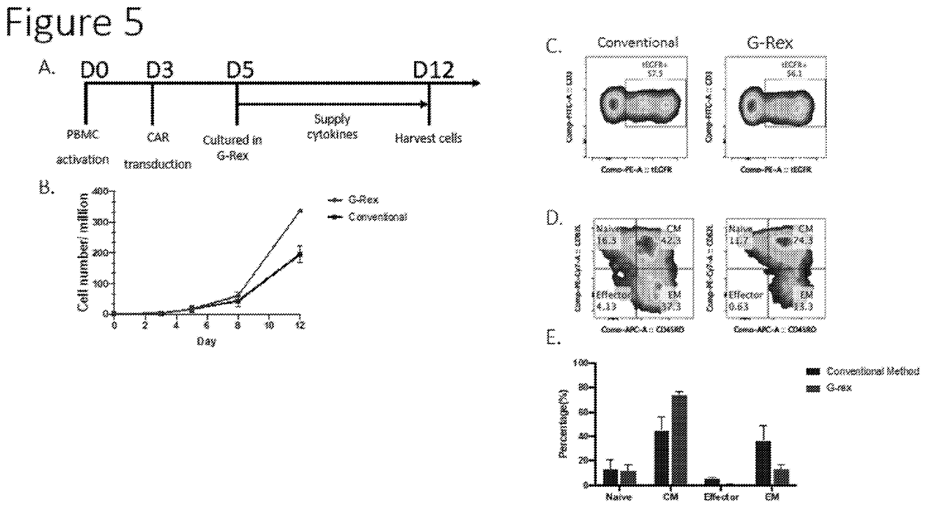

FIG. 5 shows the development of GMP-compliant CAR-T cell manufacturing method. A. Time course for CAR-T cell manufacture. Human PBMCs are activated by CD3/CD28 Dynabeads and IL7/IL15 in the flasks before retroviral transduction of CAR. Transduced cells are transferred to G-Rex plate (1×10⁶ per square metre) two days after transduction. Cytokines are replenished every two to three days until day 12. B. Cell expansion during the manufacturing procedure. Representative flow plots of CAR transduction ratio (C) and differentiation status (D) at day 12. E. Statistic of T cell differentiation. CM, central memory; EM, effector memory. Three replicates of each sample were performed. Three replicates of each sample were performed. Data are represented as mean±SEM. Data reflects typical results from four healthy individuals.

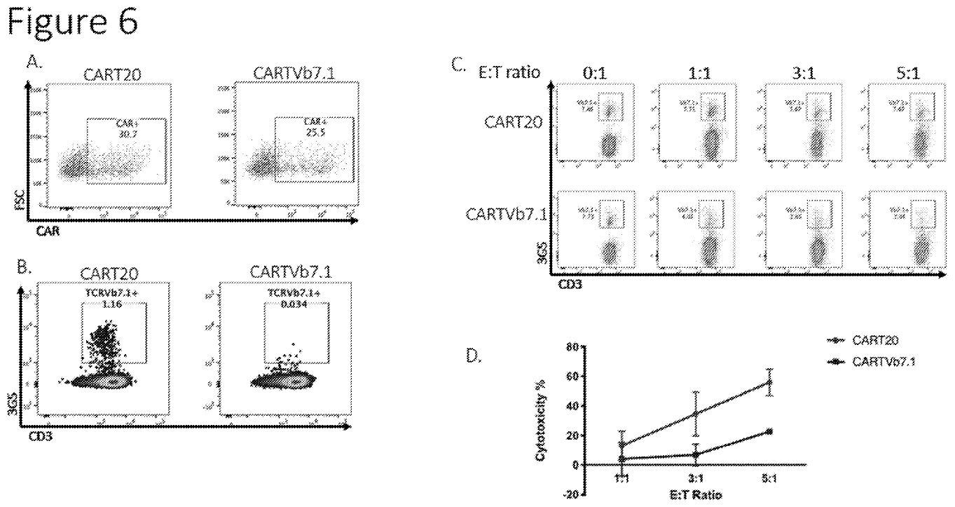

FIG. 6 shows the production and functional validation of CARTVb7.1. A. Transduced T cells were stained with an anti-EGFR antibody to detect CAR expression. Cells were propagated on CD3+single lymphocytes, and numbers indicate the percentage of tEGFR+ cells. B. Endogenous TCRVb7.1+ population detection at five days after CAR transduction. C. TCRVb7.1+primary ATL samples were stained by CFSE and mixed with a different number of effector CAR-T cells. After 6-hour incubation, cells were collected and stained by DAPI, 3G5 and CD3 antibodies for 15 minutes. A fixed volume of 5 μL Countbright beads were added into each sample. The samples were loaded to flow cytometry for absolute quantification. D. Representative result of CARTVb7.1 cells killing T tumour cells. Three replicates of each sample were performed.

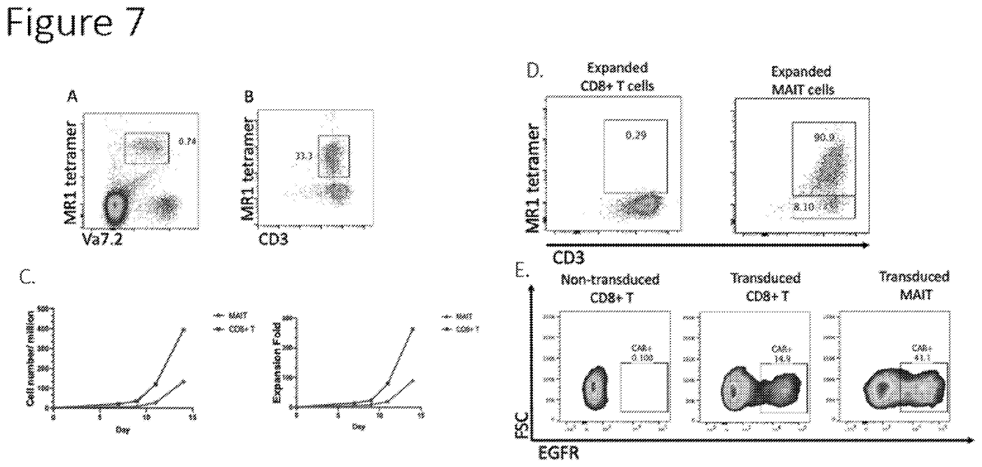

FIG. 7 shows the production of a MAIT-CART cell. A. A representative flow cytometry example of MAIT cells staining. PBMCs were stained by BV421 MR1-5-OP-RU tetramer and PE anti-TCR Vα7.2 antibody for 20 min. Cells were washed by PBS twice before being characterized by flow cytometry. B. Gating strategy for flow cytometry sorting of MAIT cells. TCR Vα7.2+ cells were isolated by magnetic separation method from PBMCs before being stained by BV421 MR1-5-OP-RU tetramer and PE anti-CD3 antibody for 20 min. Cells were washed by PBS twice before being loaded to Melody cell sorter. MR1-5-OP-RU tetramer positive population were sorted and cultured. C. Expansion curve of in-vitro cultured MAIT cells and CD8+ T cells as control. D. After 12-14 day culture, more than 90% of expanded MAIT cells were MR1-5-OP-RU tetramer specific. E. CAR transduced CD8+ T cells and MAIT cells were stained by anti-EGFR flow antibody to detect transduction efficiency.

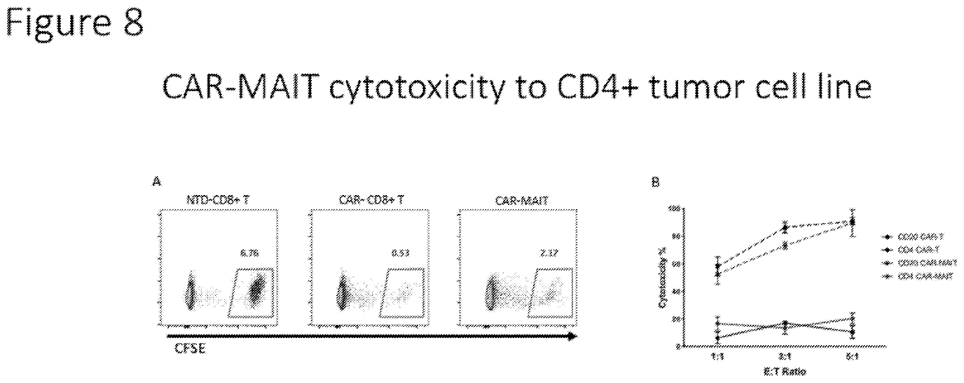

FIG. 8 shows expanded MAIT and CD8 T cells were co-cultured with CFSE-stained CD4+ cell line CEMss in E:T 0:1, 1:1, 3:1 and 5:1. Co-culture system was harvested 20 hours after incubation. The absolute quantity of survived tumour cells was counted using Countbright beads by flow cytometry analysis. (A) Flow cytometry figures of 3:1 E:T condition. (B) Statistics result of cytotoxicity. Data are represented as mean±SEM.

FIG. 9 is a map showing a first embodiment of an expression vector “CART4” used to transduce MAIT cells.

FIG. 10 is a map showing a second embodiment of an expression vector “CARTVb7.1” used to transduce MAIT cells.

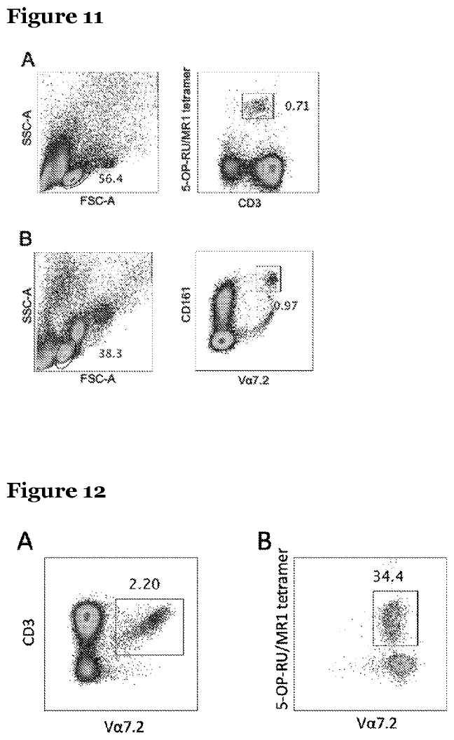

FIG. 11 shows the detection of human MAIT cells from peripheral blood mononuclear cells (PBMCs). Lymphocytes were gated, and MAIT cells were identified by their expression of CD3 and reactivity with the 5-OP-RU/MR1 tetramer (A) or expression of CD161 and TCRVα7.2 (B).

FIG. 12 shows the isolation of human MAIT cells from peripheral blood mononuclear cells (PBMCs). After separation via magnetic beads by Vα7.2 expression, Vα 7.2-positive cell population were enriched from 2.2% (A) to >97%. MAIT cells were sorted by the reactivity with the 5-OP-RU/MR1 tetramer (B).

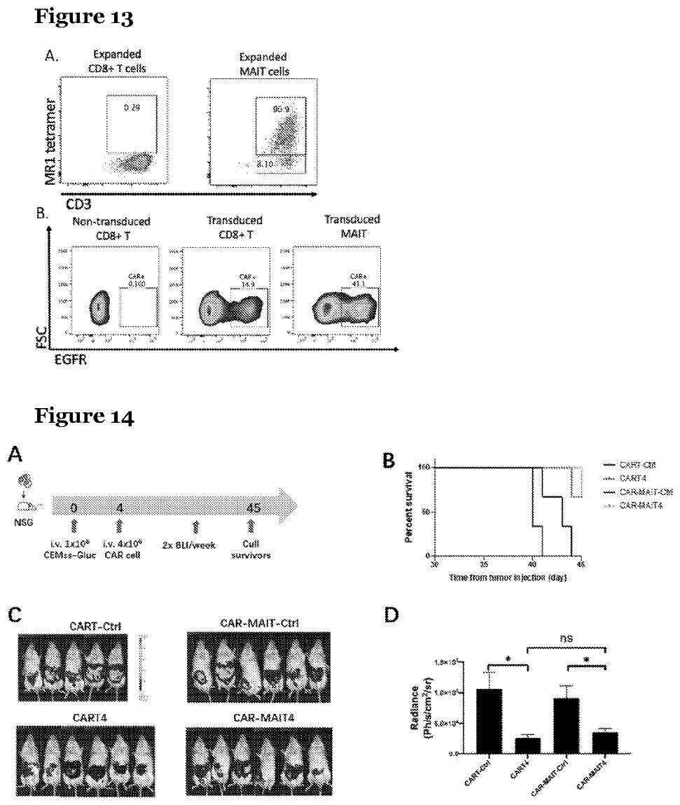

FIG. 13 shows the production of CAR-MAIT cells. After 12-14 day culture, more than 90% of expanded MAIT cells were MR1-5-OP-RU tetramer specific (A). CAR transduced CD8+ T cells and MAIT cells were stained by anti-EGFR flow antibody to detect transduction efficiency (B).

FIG. 14 shows that CAR-MAIT4 cells show efficiently anti-leukemic function in vivo. A. NSG immunodeficient mice were i.v. injected with 1×10⁶ Gluc/GFP-transduced CEM-ss cells, followed by another i.v. infusion of 4×10⁶ CAR-transduced cells. Bioluminescence imaging was performed twice per week until day 45 post tumor injection. B. Overall survival of mice treated with the indicated CAR-transduced cells by Kaplan-Meier survival analysis. C. Bioluminescence Imaging (BLI) of mice at days post tumor injection. D. Radiance of individual mice at day 40. n=5 or 6 mice per group. * P<0.05 by Student's t-test. Ph, photon; sr, steradian.

FIG. 15 shows enrichment of MAIT cells in PBMC. PBMC were stimulated by either MR1/5-OP-RU complex beads at a bead-to-cell ratio of 1:1 or 5-OP-RU antigen at 10 nM in the presence of different cytokines as indicated in the table for 6 days. The fold of MAIT cell increase was calculated by dividing the frequency of live MAIT (CD3⁺Vα7.2⁺CD161⁺) cells on day 6 by the original frequency of MAIT cells on day o. The top 5 groups were highlighted by the orange color (i.e. conditions 1, 3, 11, 12 and 13). The combination of IL-12, IL-18, and IL-23 gives the highest fold of increase of MAIT cells in the PBMCs.

### EXAMPLES

Chimeric Antigen Receptor (CAR)-based T cell therapy has achieved great success in the treatment of B-cell malignancies by targeting pan-B cell specific antigens. However, a similar strategy for T-cell lymphoma has so far been unrealised, largely due to potential severe toxicities by global T cell depletion and dysfunction/low frequency of normal T cells in T lymphoma as compared with B-cell malignancies. To overcome these limitations, the inventors engineered two novel CAR constructs, the first being referred to herein as “CART4”, which is specific to pan-T cell marker (CD4), and the second being referred to as “CARTVb7.1”, which is specific to the TCR-Vb isotype chain. Both CAR constructs incorporate one or two safety switches selected from truncated epidermal growth factor receptor (tEGFR) and inducible caspase-9 (iC9). However, as illustrated in FIG. 1, both safety switches are shown. The inventors investigated whether mucosal-associated invariant T (MAIT) cells which have low allogenic reactivity, would exhibit a similar anti-tumour killing activity of conventional T cells after transduced with the CAR construct.

In addition, it is known that MAIT cells are a subset of innate T cells defined as CD3⁺TCRVα7.2⁺CD161⁺ cells which recognise the MHC class I-like molecule, MR1. Previous studies have shown that MAIT cells can be expanded in vitro but requiring the presence of allogenic feeder cells, but this method is difficult for large-scale production and quality controls. In this study, the inventors have developed an effective method for expansion of MAIT cells in vitro by initially stimulating PBMCs with the antigen (5-OP-RU) loaded MR1 tetramer beads or 5-OP-RU alone, both in the presence of a combination of various cytokines (IL-2, IL-7, IL-15, IL-12, IL-18 and IL-23) for up to 6 days in vitro culture. The resultant MAIT cells were then isolated by MACS or FACS sorting and expanded further by anti-CD3/CD28 beads for CAR-based therapies, as described in the previous examples.

### Materials and Methods

### Construction of CAR Plasmids

The DNA fragments encoding the scFv of Hu5A8 and Leu16 and human Igk leading sequence were synthesised by Genewiz. NcoI and NotI were used to cleave these fragments as well as MSCV CAR expression retroviral vector. MSCV CAR expression vector was modified from MSCV-IRES-GFP vector (Addgene) by replacing IRES-GFP area with human CD8 transmembrane domain and third-generation CAR intracellular signalling domain (costimulatory domains of CD28 and 4-1BB, CD3ζ signalling domain). The sequence of tEGFR was obtained from U.S. Pat. No. 8,802,347B2, deleting Domain I and II of extracellular part and intracellular domains of human EGFR protein. The tEGFR was synthesised by Genewiz with the self-cleaving T2A sequence and the human granulocyte-macrophage colony-stimulating factor (GM-CSF) receptor's leader peptide. The DNA sequence of iC9 was kindly provided by Prof. Lishan Su (University of North Carolina, Chapel Hill). The DNA fragment of iC9 consists of truncated caspase 9, including its large and small subunit of caspase molecule linked to one 12-kDa human FK506 binding proteins (FKBP12) via a short Gly-Gly-Gly-Ser (GGGS) flexible linker.

### Production of Retroviral Vectors

Plat-GP cells (Cellbiolabs) were transfected with the MSCV-retroviral plasmid and pCMV-VSVG vector (Addgene) via 7 μl of X-tremeGENE HP Transfection Reagent (Roche) to produce virus with VSV envelop. To produce the high titre of CAR-encoding retroviral supernatants, a subsequent stable virus-producing cell line with PG-13 (ATCC) was performed. PG-13 cells were transduced by the viral supernatant from Plat-GP containing 8 μg/ml polybrene (Sigma). The plate was wrapped with cling-film and centrifuged at 1000 g at 32° C. for 2 hours. To produce high titre viral particle, passage the confluent cells one in two by trypsinisation. Collect the supernatant after 24 hours. Aliquot the medium and store at −80° C. after centrifuging at 300 g for 5 minutes.

### Primary T Cells and Tumour Cells

Peripheral blood mononuclear cells (PBMCs) from healthy donors were isolated by Ficoll-Paque PLUS density gradient centrifugation (GE Healthcare) for engineering CAR-T cells. T lymphoma cell lines generated from ATL or CTCL patients were cultured in D10 media (DMEM containing 10% fetal bovine serum, 100 IU/mL penicillin, 100 μg/mL streptomycin, and 2 mM L-glutamine), and T leukaemia cell lines (Jurket or CEM) were maintained in R10 media (RPMI 1640 containing 10% fetal bovine serum, 100 IU/mL penicillin, 100 μg/mL streptomycin, and 2 mM L-glutamine).

### Isolation and Expansion of MAIT Cells

MAIT cells were isolated from healthy PBMCs by a two-step method using anti-human TCR Vα7.2 antibody (Biolegend, Cat #351724) MicroBeads (Miltenyi, Cat #130-090-485) and followed by BV421 MR1-5-OP-RU tetramer kindly provided by Prof Jim Mccluskey (University of Melbourne, Australia). Briefly, the TCR Vα7.2+ T cells were isolated from PBMCs using biotinylated anti-human TCR Vα7.2 antibody and anti-Biotin MicroBeads kit according to the manufacture procedure (Miltenyi). The MAIT cells were then isolated from the TCR Vα7.2+ T cells by staining the BV421 MR1-5-OP-RU tetramers and FACS sorting using FACSMelody Cell Sorter (BD).

MAIT cells were separated from PBMCs by a two-step method. Count PBMCs cell and dilute to 1×10⁸ cells/mL in PBS/EDTA buffer in 15 mL tubes. Add 5 μL of Biotin anti-human TCR Vα7.2 antibody (Biolegend, Cat #351724) per 10⁸ cells and incubate for 20 min at 4° C. Wash cells by adding 10 times volume of PBS/EDTA buffer and centrifuge at 300×g for 10 minutes. Aspirate supernatant completely. Add 800 μL of PBS/EDTA buffer and 200 μL of Anti-Biotin MicroBeads (Miltenyi, Cat #130-090-485) per 10⁸ total cells. Mix well and incubate for 15 min at 4° C. Wash cells by adding 10 times volume of PBS/EDTA buffer and centrifuge at 300×g for 10 minutes. Aspirate 30 supernatant completely. Resuspend up to 10⁸ cells in 1 mL of PBS/EDTA buffer. Place MS column (Miltenyi) in the magnetic field of a suitable MACS Separator. Prepare column by rinsing with 500 μL of PBS/EDTA buffer. Apply cell suspension onto the column. Wash column with 3×500 μL of PBS/EDTA buffer. Elute retained cells outside of the magnetic field by adding 1 mL of PBS/EDTA buffer. Collect TCR Vα7.2+ cells, and stain with APC anti-human CD3 (Biolegend) and BV421 MR1-5-OP-RU tetramer (1:500) for 30 min at 4° C. Wash cells by adding 10 times volume of PBS/EDTA buffer and centrifuge at 300×g for 10 minutes. Aspirate supernatant completely. Add 1 mL of PBS/EDTA buffer per 108 total cells. Flow sort CD3+MR1-5-OP-RU+ cell population by FACSMelody Cell Sorter (BD).

### Activation and Expansion of MAIT Cells

Count sorted MAIT cells and resuspend in R10 medium (90% RPMI+10% FBS+1% penicillin/streptomycin+2 mM L-Glutamine) to 10⁶/mL. Activate cells with Dynabeads Human T-Activator CD3/CD28 (Life Technologies) to obtain a bead-to-cell ratio of 1:1 with 100 IU/mL IL-2 in 24-well-plate in 37° C. incubator. Two days after transduction, harvest cells and transfer cells to 6-well-plate. Refresh R10 to 0.5-1 λ10⁶/mL. Refresh cells with R10 medium every 2-3 days.

### Production of CAR-T or CAR-MAIT Cells

Purified PBMCs or MAIT cells were stimulated with Dynabeads Human T-Activator CD3/CD28 (Life Technologies) in R10 media containing 100 IU/mL IL-2 for 48 hours. The activated cells were then transfected with the retroviral virus encoding the CAR construct and cultured in R10 for another 48 hours. The CAR-T or CAR-MAIT cells were maintained in the G-Rex six-well plate (Wilsonwolf) in the presence of recombinant IL7 and IL15 (Miltenyi) for another 7 days before harvest.

### Production of CAR-T or CAR-MAIT Cells

Retroviral transduction was performed 48 hours after T-cell activation. Repeat the transduction step to achieve higher transduction efficiency 24 hours later, if necessary. 10×10⁶ cells were transferred and cultured further in G-Rex six-well plate (Wilsonwolf) with 110 mL R10 medium. Cytokines IL7/15 were replenished every two or three days. Cells were cultured in G-Rex for one week before harvest.

### Co-Culture Cytotoxicity Assay

This non-radioactive killing assay was performed as previously reported (Rowan et al., 2014). Briefly, target cells were stained with 1 uM CFSE (Biolegend) for 15 minutes at 37° C. After being washed with PBS for three times, 100,000 target cells were mixed with CAR-T or CAR-MAIT cells in ratio at 1:1, 1:3, 1:5. The 200 μl co-culture was incubated for 4 hours in the incubator at 37° C. 1 μl DAPI and 5 μl Countbright beads (BD Biosciences) were added to the samples. The samples were acquired by flow cytometry at constant speed. The number of surviving target cells was calculated as Cells in tube=(cells collected/beads collected)×total beads added to the tube.

### Intracellular Cytokine Staining

CART4 or CART20 T cells were co-cultured with specified target cells in 96-well u bottom plates. All the cells were seeded at 2×10⁵ cells/well in 200 μL/well R10 medium. T cells cultured alone as negative control, and T cells cultured with the combined stimuli of 10 μg/ml PMA and 10 μg/ml Ionomycin (Biolegend) were included as positive control. 10 μg/mL brefeldin A (Biolegend) was added to all the wells after one-hour incubation. The co-culture system was incubated for another five hours before being harvested. The cells were stained for surface markers using the antibodies for 30 minutes in the dark. The cells were fixed by using 4% paraformaldehyde solution (Biolegend) for 15 minutes at room temperature after being washed with PBS. After another wash with fix buffer, cells were resuspended by a mixture containing intracellular staining antibodies. Incubate the cells at 4° C. for 30 minutes before washing with fix buffer. They were analysed by flow cytometry with fluorescence minus one (FMO) controls, to determine the expression level of IFN-γ and TNF-α.

### In Vitro Suicide Assay

CART4 with or without iC9 cells were generated with retroviral transduction with CART4 or CART4 w/o iC9 construct. CAR-T cells were kept expanded for five to seven days after transduction. A caspase inducible drug (CID), the B/B homodimerizer AP20187 (Clontech Laboratories), was added at a various concentration to T cell culture. The induction of apoptosis induced by CID was evaluated 24 hr later using Annexin-v/7-AAD (BD Biosciences) staining and flow cytometry analysis. Survival cells were quantified by counting beads (BD Biosciences). Survival index was calculated as follows: number of living tEGFR⁺ cells/number of living tEGFR⁺ cells in untreated control samples.

### In Vivo Mouse Xenograft Experiment

6- to 8-week old NRG mice (Jackson Laboratory) were used for in vivo experiments with T leukaemia cell line. 0.5×10⁶ CEM-ss cells co-expressing Gaussia luciferase and EGFP were injected into mice via retro-orbit. Mice were randomised subsequently before T cell injection. PBMCs from healthy donors were activated and transduced to generate CART4 T cells or non-transduced T cells. On the day of transfusion, CART4 CD8⁺ T cells and non-transduced CD8⁺ T cells were negatively isolated from CART4 T cells or non-transduced T cells by using an untouched microbeads human CD8 T cell kit (Miltenyi) according to the manufacturer's instructions. 4×10⁶ isolated cells were washed by PBS twice, resuspended in 100 μl and infused to xenografted mice via retro-orbital injection. 30-50 μl peripheral blood was bled weekly via vain tail. After centrifuging at 500 g for 5 min, the plasma was separated to detect the luciferase activity following the manufacturer's instrument (Thermo Fisher Scientific). After red blood cell lysis, cells were resuspended with 100 μl antibodies master mix for surface marker staining, which contained human CD45, mouse CD45, human CD3, human CD8, human EGFR, and live/dead dye. The cell subset composition was analysed by using flow cytometry (BD AriaIII). Mice were closely monitored throughout all the studies described. The mice were euthanized when they exhibited one of the following symptoms: more than 20% loss of initial body weight, pronounced lethargy, hunched posture, severe diarrhoea or severe dermatitis.

### Statistical Analysis

Statistical analysis was performed by using GraphPad Prism software version 6.0 (GraphPad software). A two-tailed unpaired Student's t-test was used to compare data between two groups. * P<0.05, ** P<0.01, *** P<0.001. All the data with error bars are presented as mean values±standard error of the mean (SEM). A P value of less than 0.05 was considered significant. Data was analysed using GraphPad Prism software (version 8).

### 1. Detailed Methods

1.1. Detection of MAIT Cells
   1.1.1. Preparation of Human Peripheral Blood Mononuclear Cells (PBMCs)
      1.1.1.1. Transfer 5 mL of peripheral blood from volunteer donors into a heparinized tube.
      1.1.1.2. Dilute whole blood with equal volume of PBS.
      1.1.1.3. Put 5 mL of Histopaque-1077 into a 15 ml centrifuge tube. Carefully add 10 ml of diluted blood solution along the wall of the tube onto Histopaque-1077 gently; do not destroy the liquid interface.
      1.1.1.4. Centrifuge at 400×g for 30 min at room temperature.
         Note: Make sure brake and acceleration are on lowest setting on centrifuge, harsh braking and acceleration may affect layer separation.
      1.1.1.5. After centrifugation, carefully aspirate the upper layer with a Pasteur pipette to within 0.5 cm of the opaque interface containing mononuclear cells. Discard upper layer.
      1.1.1.6. Wash the harvested PBMCs twice with 10 ml of PBS by centrifugation at 250×g for 10 min.
      1.1.1.7. Resuspend the PBMC pellet with RPMI1640 culture medium.
   1.1.2. Preparation of BV421-labeled 5-OP-RU/MR1-tetramers
      1.1.2.1. Dilute 6.8 μl of streptavidin-BV421 at 0.5 mg/ml to 10.2 μl PBS and mix well.
      1.1.2.2. Add 1/10 of the streptavidin-BV421 solution (1.7 μl) to 18 μl MR1-5-OP-RU solution (5 μg) every 10 min and pipette to mix, incubating at room temperature in the dark between steps.
      1.1.2.3. Keep the BV421-label 5-OP-RU/MR1 solution at 4ºC.
         The tetramer should be titrated for use; typically 1:500 dilution is sufficient.
      1.1.3. Detection of Human MAIT Cells by Flow Cytometry
         Human MAIT cells can be detected with flow cytometry by either MR1 tetramer loaded with 5-OP-RU or by co-staining with antibodies against CD161 and TCR Vα7.2 chain. Generally, MAIT cells are 0.1-10% in peripheral blood among CD3+ T cells.
      1.1.3.1. Resuspend PBMCs at a concentration of 1×10⁶ cells per 100 μl FACS staining buffer.
      1.1.3.2. Add FACS antibodies and/or 5-OP-RU/MR1 tetramer to the samples.
         For detection of human MAIT cells, two methods can be used:
         a) Tetramer staining: use BV421-labeled human 5-OP-RU MR1 tetramer (1:500) and APC-H7-conjugated anti-human CD3 (1:200).
         b) Substitution marking: PE-conjugated anti-human TCR Vα7.2 (1:200), APC-H7-conjugated anti-human CD3 (1:200), and FITC-conjugated anti-human CD161 (1:200).
      1.1.3.3. Incubate for 30 min at 4C in the dark.
      1.1.3.4. Wash the cells with FACS staining buffer by centrifuging at 300×g for 5 min at 4C, and resuspend with 300 μl FACS staining buffer.
      1.1.3.5. Analyze MAIT cells by flow cytometry (see FIG. 11).
1.2. Isolation of MAIT Cells
   1.2.1. Magnetic Bead Separation of Vα7.2+Cells.
      1.2.1.1. Collect PBMCs, and wash the cells with Binding buffer. Discard supernatant, and resuspend cell pellets with MACS buffer at a concentration of 1×10^{7/100} μl. Add PE anti-human TCR Vα7.2 antibody (1:100). Mix evenly and incubate for 30 min on ice.
      1.2.1.2. Wash the cells with MACS buffer once by centrifuging 5 min at 300×g.
      1.2.1.3. Resuspend the cells at a concentration of 10⁷/80 μl with MACS buffer. Add μl of anti-PE microbeads per 10⁷ cells, incubate for 20 min on ice.
      1.2.1.4. Wash the cells once with 10 times volume of MACS buffer. Centrifuge at 300×g for 5 min. Resuspend in 1 ml MACS buffer.
      1.2.1.5. Prewash the MS column with 1 ml MACS buffer and assemble on the magnet.
         Apply the cells to the column and wash the column three times, each time with 1 ml MACS buffer.
   1.2.1.6. Remove the column from the magnet and elute bound cells in 1 ml MACS buffer.
      1.2.2. Flow Sorting of MAIT Cells 1.2.2.1. Collect magnet separated cells and centrifuge at 300×g for 5 min.
      1.2.2.2. Resuspend the cells at a concentration of 10⁷/100 μl with MACS buffer. Add BV421-labeled human 5-OP-RU MR1 tetramer (1:500) and APC-H7-conjugated anti-human CD3 (1:200). incubate for 20 min on ice.
      1.2.2.3. Wash the cells once with 10 times volume of MACS buffer. Centrifuge at 300×g for 5 min. Resuspend in 2 ml MACS buffer.
      1.2.2.4. Turn on the BD Prodigy Sorter and load the cell sample. Sort CD3+Tetramer+ cell population (see FIG. 12).
1.3. Activation of MAIT Cells
   1.3.1. Collect sorted MAIT cells and centrifuge at 300×g for 5 min.
   1.3.2. Discard supernatant and resuspend in R10 medium to 10⁶ cells/ml.
   1.3.3. Resuspend Dynabeads Human T-Activator CD3/CD28 by vortex for 30 sec.
   1.3.4. Transfer the desired volume of Dynabeads to a tube.
   1.3.5. Add an equal volume of buffer and mix by vortex for 5 sec. Place the tube on a magnet for 1 min and discard the supernatant.
   1.3.6. Remove the tube from the magnet and resuspend the washed Dynabeads in the R10 medium.
   1.3.7. Add desired volume of Dynabeads to cell suspension to obtain a bead-to-cell ratio of 1:1 with 100 IU/ml IL-2 in 24-well-plate in 37° C. incubator.
1.4. Retroviral transduction of MAIT Cells 1.4.1. Retroviral transduction was performed 48 hours after MAIT cell activation.
   1.4.2. One day before transduction, prepare RetroNectin coated plate. Add 15 μg RetroNectin to 1 ml PBS. Mix well and add to one well of the non-tissue culture treated 24-well-plate.
   1.4.3. Wrap the plate by fling-film and keep in 4° C. fridge overnight.
   1.4.4. On the day of gene transfer, remove unbound RetroNectin from the well. Wash twice with 2 ml PBS. Avoid the well dry.
   1.4.5. Thaw retroviral supernatant in 37° C. water bath. Transfer 1 ml of viral supernatant to each well of the RetroNectin-coated plate.
   1.4.6. Wrap the plate by fling-film and centrifuge the plate at 1000×g at 32° C. for 2 hours.
   1.4.7. During the centrifuge, collect the activated MAIT cells. Resuspend the cell by fresh R10 medium containing 100 IU/ml IL-2 to concentration of 1×106/ml.
   1.4.8. When the spin finishes, discard the supernatant from the plate. Add 1 ml of cell suspension to each well.
   1.4.9. Centrifuge the plate at 500×g for 10 min.
   1.4.10. Return the plate to in 37° C. incubator.
   1.4.11. Repeat the transduction step to achieve higher transduction efficiency, if necessary.
      Note: Transduction efficiency can be detected 48 hours after transduction by flow cytometry.
1.5. Expansion of CAR-MAIT Cells
   1.5.1. Two days after retroviral transduction, harvest cells and count cell by hemocytometer.
   1.5.2. Transfer 1×10⁷ cells to one well of Grex6M well plate. Add 130 ml of fresh R10 medium containing 100 IU/ml IL-2 and return the plate to the incubator.
   1.5.3. Refresh IL-2 to the final concentration of 100 IU/ml every three days.
   1.5.4. CAR-MAIT cells can be harvested after 8-12 days culture (see FIG. 13).
      Note: Expanded CAR-MAIT cells can be used for phenotype test, functional assay or be froze in liquid nitrogen.

### Results

### Example 1—Generation of CD4-Targeting T Cells and TCR Vbeta 7.1-Targeting T Cells

Referring to FIG. 1A(1) and (2), there are shown schematic maps illustrating the functional elements included in two different embodiments of a CAR construct according to the invention.

In each embodiment, the construct is flanked by upstream and downstream long terminal repeats (LTR). A 5′ promoter is disposed downstream of the 5′ LTR, and can be the PGK promoter. Disposed 3′ of the promoter, there is SP, a Igk signaling peptide, for leading the fusion protein to the T-cell outer membrane. Disposed 3′ of the SP there is provided a scFv region including an upstream VL (variable light chain) sequence, a central G4S sequence, and a downstream VH (variable heavy chain) sequence. The VL and VH sequences can, in one embodiment (as shown in FIG. 1A(1)), be Hu5A8 light chain variable region and heavy chain variable region for binding CD4 antigen. In the other embodiment (as shown in FIG. 1A(2)), the VL and VH sequences can be 365 light chain variable region and heavy chain variable region for binding TCR-Vb7.1.

Disposed 3′ of scFv region, there is a CD8a hinge and transmembrane (TM) domain structure domain for CAR display and anchoring. Disposed 3′ of the hinge and TM, there is provided an intracellular domain, including a signaling domain of CD28, 4-1BB and CD3ζ chain, for triggering the intracellular signaling pathway. A P2A self-cleavage peptide is disposed 3′ of the ζ chain, and 5′ of a truncated version of EGFR (EGFRt), for tracking and to act as a first safety switch. A second P2A self-cleavage peptide is disposed 3′ the EGFRt, and 5′ of inducible Caspase-9 (iC9), which acts as a second safety switch. The construct includes a woodchuck hepatitis regulatory element (WPRE)—see FIGS. 9 and 10 plasmid—which enhances expression, and finally a terminal 3′ LTR.

The DNA sequence of the mouse IgG antibodies humanised 5A8 (Hu5A8) with immunospecificity towards human CD4 was found from CN103282385. Hu5A8, also known as TNX-355 or ibalizumab, was widely assessed in Phase I and Phase II clinical trials for inhibiting HIV entry by blocking the HIV-binding site of CD4 molecule. As a control, the VH chain and VL chain of an anti-CD20 monoclonal antibody (Leu16) was also synthesized, which had been evaluated for its efficacy in pre-clinical and clinical CAR-T studies. The scFv fragments were cloned into the backbone of a third-generation CAR plasmid in frame with a CD8 transmembrane domain, a CD28 endodomain, a 4-1BB endodomain and the CD3ζ chain. A third-generation CAR was used due studies demonstrating its superiority over first- and second-generation CAR. The utility of tEGFR was examined as a selection, in vivo tracking marker, and also as a first safety switch, for ablation of engineered CAR-T cells. Thus, this residual tEGFR sequence was linked with CAR sequence by the T2A-ribosomal skip sequence.

CAR-T cells can remain in the patients sometimes as long as dozens of years as in the case of the anti-CD19 and anti-HIV CAR trials. Unlike B-cell aplasia, long-term CD4+ T-cell aplasia is life-threatening. Therefore, it is necessary to establish the safety methods to remove the CART4 cells of the invention from patients after tumour or virus depletion, or in emergency cases due to severe side effects during CAR-T therapy. The dimerization drug-induced Caspase-9 (iC9) suicide switch is based on the fusion of human caspase-9 to a mutated human FK506-binding protein (FKBP), which allows conditional dimerization in the presence of a small chemical molecule drug, AP20187, referred to as a caspase inducible drug (CID). The use of iC9 has already been proven to be safe and effective in a clinical trial of haploidentical HSC transplantation. Therefore, the gene fragment containing CD4 CAR, tEGFR and iC9 was then synthesized (FIG. 1A.1) and inserted in a retroviral MSCV (murine stem cell virus) vector (shown in FIG. 9; 10,348 bp). Also, the gene fragment containing TCR Vbeta7.1 CAR, tEGFR and iC9 was then synthesized (FIG. 1A.2) and inserted in a retroviral MSCV (murine stem cell virus) vector (shown in FIG. 10; 10,347 bp).

In order to demonstrate co-expression of CAR and tEGFR, human PBMCs were activated with Dynabeads Human T-Activator and genetically engineered by retroviral transduction of the plasmids shown in FIGS. 9 and 10 to express the CAR/tEGFR/iC9 gene construct. Indeed, expression levels of CAR and the tEGFR were found to be tightly correlated, as shown by the detection of double-positive cell populations upon surface staining with a mouse scFv-specific anti-mouse IgG F(ab′)2 antibody in combination with an EGFR-specific antibody (FIG. 1B). Along with T cell expansion, tEGFR⁺ cell population maintained its proportion counting ˜50% of total cells. (FIG. 1C).

In order to evaluate the efficiency of iC9 safety switch in vitro, a CART4 variation without the iC9 gene (CART4 w/o iC9) was cloned as a control. T cells transduced with CART4 or CART4 without iC9 construct were exposed to increasing concentrations of the CID AP20187 (0.1 nM to 100 nM) for 24 hours. Cell death was accessed by flow cytometry analysis with 7AAD and Annexin-V. The tEGFR-positive percentage in the survived population dropped along with the increasing concentration of the CID. 69.1% of tEGFR high cells were eliminated after a single 100 nM dose of CID (FIG. 1D). Consistent with the observations from other studies, the cells that escape killing were those expressing low levels of the transgene with a 50% reduction in mean fluorescence intensity (MFI) of tEGFR after CID (FIG. 1E). Therefore, the non-responding T cells expressed insufficient iC9 for functional activation of CID. For clinical applications, CAR-T cells may have to be sorted for sufficient transgene expression before administration.

### Example 2—Functional Validation of CART4 T Cells In Vitro

Within four days after CAR transduction, the CD4⁺ T cells were almost completed depleted as compared with non-transduced (NTD) and CART20 control, in which about 45% of cells remained CD4-positive (FIG. 2A). These data indicated the potent activity against CD4 of CART4 cells during T cell expansion.

Co-cultures were established against autologous primary healthy donor PBMCs. CFSE-labelled autologous PBMCs were co-cultured with either CD8⁺CART4 cells or CART20 cells. In both settings, CART4/20 cells mediated high-level cytotoxicity against respective target cells. 94% of CD4+ cells in PBMCs were lysed by CART4 cells in the condition of E: T ratio 3:1 during 4-hour co-culture. However, there was no specific T-cell cytotoxicity of CART4 in response to CD20+ cells, compared with NTD T cells (FIG. 2C).

To further evaluate the function of CART4 cells, the inventors tested the anti-tumour efficacy of CART4 cells using the Jurkat cell line and CEM-ss cell line. Jurkat and CEM-ss cell lines were T-cell lines initially established from the peripheral blood of patients with T-cell leukaemia or human T4-lymphoblastic leukaemia. Both of the cell lines express CD4, while the CEM-ss cell line expresses a higher level of CD4 (FIG. 2D). 10) Indeed, CART4 cells targeted T tumour cell lines based on CD4 expression level. After short-term incubation, CART4 cells successfully eliminated CEM-ss cells at the E: T (effector: target) ratio of 5:1. As a control, CART4 cells were also tested for their activity to CD4-lymphoma cells, a human B-cell line (BCL) that does not express CD4 (FIG. 2D). Flow cytometry analysis demonstrated that CART4 cells were unable to target BCL (FIG. 2E). Moreover, CART4 cells cultured with CD4+tumour cells exhibited significant IFN-γ and TNF-α responses by intracellular cytokine staining (FIG. 2F). Therefore, these data proved a strong dose-dependent response of CART4 against CD4 expression. When CART4 cells were incubated with CD4-negative cells, no killing effect was observed. These results therefore show that CART4 cell ablation is specific to CD4.

### Example 3—CART4 Cells Specifically Kill CD4+T Tumour Cells

To examine the function of CART4 to patient samples, PBMCs from ATLL patients were thawed and phenotyped. All the samples had a range of CD4 expression from 67.4% to 97.7%. Most of the CD4⁺ cells express one unique ß chain of the T cell receptor (TCR VB) indicating the clonal development of T cell leukaemia202-204 (FIG. 3A). As quantified by flow cytometry analysis, co-culture of ATLL patient samples with CART4 cells for 4 hours resulted in rapid and definitive ablation of CD4+malignancies. About 80% ablation was observed for all ATLL co-cultures, consistent with the ablation of blast T cell lines previously shown (FIG. 3B). Studies were also conducted using samples from six CTCL patients. Similarly, observed robust cytotoxicity of CART4 cells against freshly thawed primary CTCL cells was observed, resulting in about 60%˜80% reduction of malignant T cells after 4 hours of co-culture (FIG. 3C). Therefore, CART4 cells efficiently eliminated aggressive CD4+T-malignancies directly isolated from patients samples. These results indicate that CD4 is a promising therapeutic target for CD4⁺T-malignancy.

### Example 4—CART4 Cells Efficiently Mediate Anti-Leukemic Effects In Vivo

In order to evaluate in vivo antitumor activities, the inventors developed a xenogeneic mouse model using the Gaussia luciferase-expressing CEM-ss cell line. They first tested ability of the CART4 cells to delay the appearance of leukaemia in the NRG mice with a single dose (4×10⁶) of CART4 cells. Before the injection, about 50% of cells expressed the anti-CD4 CAR as demonstrated by flow cytometry analysis. Mice received retro-orbital injections of CEM-ss cells. Four days after tumour engraftment, a single dose of retro-orbital injection of CART4 cells or NTD CD8⁺ T cells was administered to leukaemia-bearing mice (FIG. 4A). Tumour burden was monitored by measuring luciferase activity in peripheral blood weekly. CART4 cells infused provided robust protection against leukaemia progression (FIG. 4B) and significantly extended median survival of the mice (38 days in the control group vs 60 days in the CART4 group, P=0.026 by Mantel-Cox log-rank test) (FIG. 4C). Indeed, by the endpoint, eGFP+tumour progression was dramatically delayed in spleens and bone marrows by flow cytometry analysis (FIG. 4D).

Although relapsed tumour cells retained expression of CD4, the expressing level dropped up to about 40% MFI compared to control group (FIG. 4E). This downregulation, however, was insufficient to compromise the ability of CART4 cells to eliminate the relapsed tumour (FIG. 4F). This result was indicating that a lack of CAR-T cell persistence rather than antigen escape was the primary reason for the tumour relapse.

### Example 5—Development of GMP-Compliant CAR-T Cell Manufacturing Method

To assess scalability and simplify CAR-T cell manufacturing, an optimized standard operating procedure was established using a gas-permeable static cell culture system (G-Rex) for CAR-T manufacture (FIG. 5A). G-Rex system contains a silicone membrane at the bottom of the plate. Gas exchange, including O₂ and CO₂ across the membrane, allows an increased depth of the culture medium, providing more nutrients 30 and diluting waste. PBMCs were activated and transduced, and 10×10⁶ cells were transferred and cultured further in G-Rex six-well plate. The cells were replenished with the cytokines IL-7 and IL-15 every two to three days. Cells expanded to more than 3×10⁸ from initial number of 2×10⁶ cells, with an increase of 150-fold over 15 days (FIG. 5B). Next, the transduction efficiency of the final product following T cell expansion in the G-Rex system was ascertained. The final transduction efficiency of CART4 was 57.6%+7.1%, similar to the cells produced from the conventional flask (53.7%+5.3%), as shown in FIG. 5C. As expected, endogenous CD4+ population was depleted entirely in the final product, indicating the anti-CD4 activity of the CAR-T cells.

Interestingly, CAR-T cells produced in the G-Rex exhibited differentiation preference towards central memory phenotype. Evaluation of the memory markers CD45RO and CD62L, showed higher CD45RO CD62L double-positive population percentage (77%±7.1% vs 41%±5.5%), compared with cells cultured in conventional culture flask (FIG. 5D). CD45RO CD62L double-positive cells were central memory T cells, which are considered to be required for long-term persistence in vivo. Thus, this bioprocess optimization method increased the cell output and the proportion with a central memory phenotype while decreasing the number of technician interventions and cost of CAR-T manufacture.

### Example 6—Generation of TCR Vβ7.1-Specific CAR-T Cells

T cell malignancies are usually developed from one monoclonal cancerous cells expressing unique TCR. A broad array of antibodies directed against the variable (V) region of the TCR B (VB) chain has become available in a directly conjugated multicolour format that permits assessment of 22 of 25 VB families, covering 75% of the normal circulating T-cell repertoire. Therefore, the inventors consider TCR Vβ is a potential target of CAR-T therapy towards T cell malignancies. To develop TCR Vβ targeting CAR-T (CARTVb7.1) cells, the inventors cloned scFv region of a hybridoma cell 3G5, which produces monoclonal antibody specific to human TCR VB 7.1 (Dr Margret Callam from Andrew's lab, Oxford), to the CAR construct, as shown in FIG. 1A(2). Five days after CAR transduction, the endogenous TCR Vβ7.1+ population were almost completed depleted as compared with CART20 control, in which about 1.2% of cells remained TCR Vβ7.1-positive (FIG. 6B). These data indicated the potent activity against TCR Vβ7.1 of CAR-T cells during T cell expansion.

To further evaluate the function of CARTVb7.1 cells, the inventors tested the anti-tumour efficacy using tumour cells isolated from a ATL patient, who was diagnosed with a TCRVβ7.1-positive tumour. Indeed, as quantified by flow cytometry analysis, co-culture of ATL patient samples with CARTVb7.1 cells for 6 hours resulted in rapid and definitive ablation of CD4+malignancies. About 60% ablation was observed for all ATL co-cultures (FIG. 6C, D). These results indicate that TCR VB is a promising therapeutic target for T-malignancy.

### Example 7—Development of CAR-MAIT Cells

Currently, most of the CAR-T therapies utilize autologous conventional CD3+ T cells. However, immune cells from cancer patients may be poorly functional or present in a low number. In particularly, it would be risky to expand and genetically modify PBMCs of T-malignancy patients, as it's possible to engineer tumour cells with a CAR. Therefore, it's desirable to develop an immunotherapy in which third party, allogeneic cell could be manufactured. Here, the inventors developed a two-step method to isolate mucosal-associated invariant T cells (MAIT cells) from PBMCs by combination of magnetic separation and flow cytometry sorting. After the first step separation based on TCR Vα7.2 expression, MAIT cell percentage was increased from 0.74% to 33.3% (FIG. 7A, B). The next step flow sorting could further increase the MAIT purity to 95%. The sorted cells were activated with Dynabeads Human T-Activator CD3/CD28 and expanded in the presence of a cocktail of cytokines (IL-2, IL-7, and IL-15). The expansion method yielded about 100-fold expansion within 12-14 days (FIG. 7C). At the harvest time, 90.9% of expanded cells maintained their specificity to MR1-5-OP-RU tetramer (FIG. 7D). Also, the expanded MAIT cells could be successfully engineered by CAR gene by retroviral transduction (FIG. 7E). CAR transduced MAIT (CAR-MAIT) cells possess comparable cytotoxicity capacity as conventional CAR-T cells (FIG. 8).

### Example 8—CAR-MAIT Cells Efficiently Mediate Anti-Leukemic Effects In Vivo

To evaluate the anti-tumour function of CAR-MAIT cells in vivo, the inventors tested ability of the anti-CD4 CAR-MAIT (CAR-MAIT4) cells to delay the progression of leukaemia in the NSG mice with a single dose (4×10⁶) of CAR cells. Mice received intravenous injections of CEM-ss cells. Four days after tumour engraftment, a single dose of intravenous injection of CAR-MAIT4 cells or CART4 cells were administered to leukaemia-bearing mice (FIG. 14A). Anti-CD20 CAR-MAIT (CAR-MAIT-Ctrl) cells or anti-CD20 CART (CART-Ctrl) cells were administrated as control groups. Tumour burden was monitored by measuring luciferase activity weekly. CAR-MAIT4 cells and CART4 cells infused provided comparable protection against leukaemia progression (FIG. 14C and D) and significantly extended survival of the tumour-bearing mice (FIG. 14B).

### Example 9—Detection, Isolation, Expansion, and Engineering of Human MAIT Cells

Using the methods described herein, human MAIT cells were detected, isolated, expanded and engineered.

As shown in FIG. 11, human MAIT cells with analysed by flow cytometry.

As shown in FIG. 12, the MAIT cells were flow sorted.

The MAIT cells were then activated, and transduced with the CAR-expressing vectors to create CAR-MAIT cells, which were then expanded as shown in FIG. 13.

### Example 10—Expansion of MAIT Cells by Stimulating PMBCs

MAIT cells are a subset of innate T cells defined as CD3⁺TCRVα7.2⁺CD161⁺ cells which recognise the MHC class I-like molecule, MR1. Previous studies have shown that MAIT cells can be expanded in vitro but requiring the presence of allogenic feeder cells, but this method is difficult for large-scale production and quality controls. In this study, the inventors have developed a highly novel and effective method for expansion of MAIT cells in vitro by initially stimulating PBMCs with the antigen (5-OP-RU) loaded MR1 tetramer beads or 5-OP-RU alone, both in the presence of a combination of various cytokines (IL-2, IL-7, IL-15, IL-12, IL-18 and IL-23) for up to 6 days in vitro culture. The resultant MAIT cells were then isolated by MACS or FACS sorting and expanded further by anti-CD3/CD28 beads for CAR-based therapies, as described in the previous examples.

### Material and Methods

### 1. PBMC Isolation

PBMCs were isolated from buffy coats of healthy blood donors via centrifugation on a Ficoll-Hypaque density gradient. Aliquots of the isolated PBMCs were frozen and stored in liquid nitrogen until used. Before starting the experiments, frozen PBMC stocks were thawed and incubated at 37° C. in RPMI medium supplemented with 10% FBS.

### 2. Preparation of MR1/5-OP-RU Complex Beads

MR1/5-OP-RU tetramer-coated beads were generated by using the M-280 dynabeads with Streptavidin from ThermoFisher and the biotinylated MR1 monomers. The 5-OP-RU loaded MR1 monomers were kindly provided by Dr Jim Mccluskey (University of Melbourne, Australia). The beads were mixed and coated with 5-OP-RU-loaded MR1 monomers (5 μg/3×10⁷ beads) for 12 h at 4° C. on a rocker. Excess unbound protein was removed by two 10-min washes in PBS. The prepared MR1 tetramer-coated beads were resuspended in PBS and stored at 4° C. until use.

### 3. Enrichment of MAIT Cells in PBMCs

PBMCs (2×10⁵ cells per well) were cultured in 96-well plates containing R10 medium (90% RPMI+10% FBS+1% penicillin/streptomycin+2 mM L-Glutamine) in 37° C. incubator and stimulated by either MR1/5-OP-RU complex coated beads at a bead-to-cell ratio of 1:1 or purified 5-OP-RU antigen (10 nM) (provided by Dr Jeffrey Mak, University of Queensland, Australia) in combination with different cytokines for 6 days in vitro. Cytokines IL-2 (100 IU/ml) (Roche), IL-7 (50 ng/ml) (Miltenyi), IL-15 (50 ng/ml) (Miltenyi), IL-12 (50 ng/ml) (Miltenyi), IL-18 (50 ng/ml) (ThermoFisher) and IL-23 (50 ng/ml) (Miltenyi) were added in 15 different combinations, numbered 1 to 15, as indicated in the table in FIG. 15. On day 6, the expanded cells were collected and analyzed to determine the percentage of MAIT cells by flow cytometry as described below.

### 4. FACS Analysis of MAIT Cell Frequency in PBMCs

The expanded PBMCs were stained for surface markers using the antibodies for 30 minutes in the dark. FITC-conjugated CD3 (clone BW264/56, Miltenyi), PE-conjugated Vα7.2(clone 3C10, Biolegend), APC-conjugated CD161 (Clone DX12, BD) were used at 1:100 to label the cells. MAIT cells are defined as CD3⁺Vα7.2⁺CD161⁺ cells. Dead cells were excluded using the LIVE/DEAD™ Fixable Aqua Dead Cell Stain Kit

(ThermoFisher). Stained cells were washed with five to ten-volume of PBS for centrifuge at 500 xg for 5 minutes and resuspended with 200 μl PBS before flow cytometry analysis. The flow cytometry results were analyzed by Flow.Jo.

### Results

Referring to FIG. 15, there is shown the results of enriching MAIT cells in PBMC. PBMC were stimulated by either (i) MR1/5-OP-RU complex beads at a bead-to-cell ratio of 1:1 or (ii) 5-OP-RU antigen at 10 nM—each in the presence of different cytokines (IL-2, IL-7, IL-15, IL-12, IL-18 and IL-23) as indicated in the table for 6 days. For example, condition 1 corresponds to IL-2 only, condition 2 corresponds to IL-7 and IL-15, and condition 3 corresponds to IL-2, IL-12 and IL-18, and so on.

The fold of MAIT cell increase was calculated by dividing the frequency of live MAIT (CD3⁺Vα7.2⁺CD161⁺) cells on day 6 by the original frequency of MAIT cells on day 0. The top five groups were highlighted by the orange color (i.e. conditions 1, 3, 11, 12 and 13).

As can be seen, for MR1/5-OP-RU complex beads (for Donor 1), the cytokine combination of 1, 13, 12, 3 and 11 gave the highest fold of increase of MAIT cells in the PBMCs. For MR1/5-OP-RU complex beads (for Donor 2), the cytokine combination of 12, 13, 1, 11 and 3 gave the highest fold of increase of MAIT cells in the PBMCs.

As can be seen, for 5-OP-RU (for Donor 1), the cytokine combination of 3, 1, 12, 13 and 11 gave the highest fold of increase of MAIT cells in the PBMCs. For 5-OP-RU (for Donor 2), the cytokine combination of 8, 13, 12, 11 and 3 gave the highest fold of increase of MAIT cells in the PBMCs.

Based on these data, it is clear that different cytokines and combinations of the cytokines (IL-2, IL-7, IL-15, IL-12, IL-18 and IL-23) resulted in differing levels of stimulation resulting in improved enrichment of MAIT cells in PBMC. Overall, the combination of IL-12, IL-18, and IL-23 gives the highest fold of increase of MAIT cells in the PBMCs.

### DISCUSSION

MAIT cells are a subset of innate T cells defined as CD3+TCRVα7.2+CD161+ cells which recognise the MHC class I-like molecule MR1. Previous study has shown that MAIT cells can be expanded in vitro but requiring the presence of allogenic feeder cells—a method is difficult for a large-scale production and quality controls. As described above, the inventors we have developed a surprisingly effective method for expansion of MAIT cells in vitro by initially stimulating PBMCs with the antigen (5-OP-RU) loaded MR1 tetramer beads or 5-OP-RU alone in the presence of a combination of three cytokines (IL-12, IL-18 and IL23) for up to 6 days in vitro culture. The MAIT cells were then isolated by MACS or FACS sorting and expanded further by anti-CD3/CD28 beads for CAR-based therapies.

Currently, no well-established treatment for T-cell lymphoma is available as compared with B-cell malignancies, with the only potential curative regimen being allogenic haematopoietic stem cell transplantation (HSCT), which in itself has significant treatment-associated mortality. Because most (>95%) of T-cell lymphoma is derived from a dominant T cell clone expressing a defined T-cell receptor (TCR) gene (i.e. clonal TCR-Vb chain) and pan-T help cell marker CD4, monoclonal antibodies targeting these markers have been explored for treatments of T-cell lymphoma and some resulted in partial regression in small clinical trials(d′Amore et al., 2010; Hagberg et al., 2005; Kim et al., 2007).

Despite the CAR-T is a very effective treatment for B-cell malignancies by targeting pan-B cell marker CD19, this approach has encountered significant obstacles when applying for treatment of T-cell lymphoma. Firstly, unlike B-cell depletion, persistent T-cell aplasia, particularly CD4+ T-cell depletion, would result in severe toxicity, such as the opportunistic infections observed during chronic HIV infection. Secondly, T-cell lymphoma-associated impaired T cell function and lower normal T cell count caused by a dominant T cell tumour growth cannot be used for generating autologous CAR-T cells, therefore allogenic CAR-T cells are needed for treatment of T-cell lymphoma. Finally, most T-cell lymphoma are solid tumours associated with lymph nodes and skin tissues which are difficult to treat by conventional CAR-T due to their lower tissue infiltrating capability as well as the hostile tumour microenvironment.

To address these issues, the inventors designed a CAR-targeting CD4 antigen (CART4) containing tEGFR and iC9 as safety switches to selectively eliminate the CART4-transduced T cells after eradicating the tumour cells, allowing the recovery of normal CD4⁺ T cells from autologous hematopoietic stem cells or allogenic HSCT. The transit depletion of CD4⁺T cells has been shown to be safe and tolerable in the treatment of autoimmune diseases by anti-CD4 antibodies (Hagberg et al., 2005; Kim et al., 2007). The CART4-tranduced human T cells were able to kill CD4+ T-cell lymphoma cell lines isolated from ATLL or CTCL patients in vitro and inhibit the tumour growth in vivo in mouse xenograft model. More importantly, these CART4+ T cells co-express the CAR with both tEGFR as detected by anti-EGFR antibodies and iC9 as determined by the CID drug-induced apoptosis of CART4+ T cells in vitro and in vivo. The expression of tEGFR could be used for either monitoring the CART4+ T cell proliferation or eliminating the CART4+ T cells with anti-EGFR antibodies in vivo.

In general, normal T cells consist of a highly diverse TCR repertoire to maintain cellular immunity against pathogen infections. The TCR consists of a heterodimer of the a and b chains containing N-terminal variable and C-terminal constant regions. The TCR-Vb regions (chains) are more polymorphic than the TCR-Va, and often used for analysing clonality of immune responses or T cell malignancies. Currently, there are 22 mAbs specific to TCR-Vb chain family covering 75% TCR repertoire. As most of T-cell lymphoma are derived from single T cell clone expressing the same TCR Vb chain, therefore a CART targeting TCR-Vb chain defined to a tumour clone while preserving the rest of normal T cell repertoire would be an ideal approach to minimise the opportunistic infections, and there would be no need to remove the CART cells after transfusion.

As proof-of-concept for anti-TCR-Vb based immunotherapy, the inventors engineered a CAR specifically targeting TCR-Vb 7.1 chain (CARTVb7.1) and showed that CARTVb7.1 transduced T cells were able to effectively eliminate the TCR-Vb 7.1 positive tumour cells isolated from a ATL patient, suggesting that anti-TCR-Vb CAR can provide an alternative immunotherapy for T-cell lymphoma.

Finally, the inventors investigated whether MAIT cells could be used as the effector cells for CAR-based therapy, because MAIT cells have several advantages over the conventional T cells, including:
(1) low allogenic reactivity (i.e. inducing graft vs host disease, GVHD) due to expressing an invariant TCR highly conserved during mammalian evolution;
(2) regulatory functions including the inhibition of GVHD in mice models;
(3) killing activities with activation inducing GrB, perforin, and GrA; and
(4) tissue homing such as distribution in gut mucosal, skin, and lung.

As described herein, the CAR-transduced MAIT cells (CAR-MAIT) showed at least comparable anti-tumour activity in vitro and in vivo as conventional CAR-T cells did. In conclusion, the inventors have developed a novel CAR-MAIT-based immunotherapy for effective treatment of T-cell malignancy by targeting either a pan-T cell marker CD4 with switchable CAR-T to reduce on-target/off-tumour toxicity and cytokine release syndrome or specific TCR-Vb chain, which is unique to the malignant T cells to avoid the global immunosuppression. More importantly, the CAR-MAIT cells may have the potential to develop an allogenic CAR-based therapy which is required for the treatment of T-cell lymphoma. If manufactured consistently, i.e. a massive ex vivo expansion, they could be used for off-the-shelf development. The inventors believe that the CAR-MAIT may provide a new approach for effective therapy not only for T-cell malignancies, but also for other non-immune cell type of tumours.

### CONCLUSIONS

Chimeric Antigen Receptor (CAR)-based T cell therapy has achieved great success in the treatment of B-cell malignancies by targeting pan-B cell specific antigens. However, a similar strategy for T-cell lymphoma has so far been unrealised, largely due to potential severe toxicities by global T cell depletion and dysfunction/low frequency of normal T cells in T lymphoma as compared with B-cell malignancies. To overcome these limitations, the inventors engineered a novel CAR construct specific to pan-T cell marker (CD4) or TCR-Vb isotype chain, incorporating two safety switches: truncated epidermal growth factor receptor (tEGFR) and inducible caspase-9 (iC9). The inventors investigated whether mucosal-associated invariant T (MAIT) cells which have low allogenic reactivity, would exhibit a similar anti-tumour killing activity of conventional T cells after transduced with the CAR construct.

Surprisingly, the CAR transduced T cells not only showed a specific killing of CD4+T lymphoma cells or the TCR-Vb specific T leukaemia clone isolated from the patients, but also were eliminated upon treatment with the inducing agent in vitro and in vivo. Furthermore, the inventors have shown for the first time that the CAR-MAIT cells are able to inhibit the tumour growth as efficiently as the conventional T cells in vitro and in vivo. This study provides a novel strategy for the treatment of T cell lymphoma.

Thus, the Mucosal-associated invariant T (MAIT) cells of the invention, a type of immune cells known for their involvement in a broad range of infectious and non-infectious diseases and their unusual specificity for microbial riboflavin-derivative antigens presented by the major histocompatibility complex (MHC) class I-like protein MR1, are developed into a novel form of immunotherapy to treat patients with cancer by genetically modified MAIT cells with a chimeric antigen receptor (CAR) that enables them to specifically recognize and attack T lymphoma.

### REFERENCES

d′Amore, F., Radford, J., Relander, T., Jerkeman, M., Tilly, H., Osterborg, A., Morschhauser, F., Gramatzki, M., Dreyling, M., Bang, B., & Hagberg, H. (2010). Phase II trial of zanolimumab (HuMax —CD4) in relapsed or refractory non-cutaneous peripheral T cell lymphoma. British Journal of Haematology, 150(5), 565-573. https://onlinelibrary.wiley.com/doi/full/10.1111/j.1365-2141.2010.08298.x
Hagberg, H., Pettersson, M., Bjerner, T., & Enblad, G. (2005). Treatment of a patient with a nodal peripheral T-cell lymphoma (Angioimmunoblastic T-cell lymphoma) with a human monoclonal antibody against the CD4 antigen (HuMax-CD4). Medical Oncology (Northwood, London, England), 22(2), 191-194. https://link.springer.com/article/10.1385/MO:22:2:191
Katsuya, H., Ishitsuka, K., Utsunomiya, A., Hanada, S., Eto, T., Moriuchi, Y., Saburi, Y., Miyahara, M., Sueoka, E., Uike, N., Yoshida, S., Yamashita, K., Tsukasaki, K., Suzushima, H., Ohno, Y., Matsuoka, H., Jo, T., Amano, M., Hino, R., . . . Project, A.-P. I. (2015). Treatment and survival among 1594 patients with ATL. Blood, 126(24), 2570-2577. https://ashpublications.org/blood/article/126/24/2570/34701/Treatment-and-survival-among-1594-patients-with
Kim, Y. H., Duvic, M., Obitz, E., Gniadecki, R., Iversen, L., Osterborg, A., Whittaker, S., Illidge, T. M., Schwarz, T., Kaufmann, R., Cooper, K., Knudsen, K. M., Lisby, S., Baadsgaard, O., & Knox, S. J. (2007). Clinical efficacy of zanolimumab (HuMax-CD4): two phase 2 studies in refractory cutaneous T-cell lymphoma. Blood, 109(11), 4655-4662. https://ashpublications.org/blood/article/109/11/4655/23083/Clinical-efficacy-of-zanolimumab-HuMaxCD4-two
Park, J. H., Rivière, I., Gonen, M., Wang, X., Sénéchal, B., Curran, K. J., Sauter, C., Wang, Y., Santomasso, B., Mead, E., Roshal, M., Maslak, P., Davila, M., Brentjens, R. J., & Sadelain, M. (2018). Long-Term Follow-up of CD19 CAR Therapy in Acute Lymphoblastic Leukemia. New England Journal of Medicine. https://doi.org/10.1056/nejmoa1709919
Rowan, A. G., Suemori, K., Fujiwara, H., Yasukawa, M., Tanaka, Y., Taylor, G. P., & Bangham, C. R. M. (2014). Cytotoxic T lymphocyte lysis of HTLV-1 infected cells is limited by weak HBZ protein expression, but non-specifically enhanced on induction of Tax expression. Retrovirology, 11(1), 112-116. https://retrovirology.biomedcentral.com/articles/10.1186/s12977-014-0116-6

### CLAUSES

1. A mucosal-associated invariant T (MAIT) cell expressing a chimeric antigen receptor (CAR).
2. A MAIT cell according to clause 1, wherein the CAR-MAIT cell expresses a CAR which targets a CD4 antigen on a T-cell.
3. A MAIT cell according to clause 2, wherein the CAR is specific for a CD4 antigen which comprises an amino acid substantially as set out in SEQ ID No:1, or a variant or fragment thereof.
4. A MAIT cell according to any preceding clause, wherein the CAR-MAIT cell expresses a CAR which targets a T-cell receptor (TCR) beta-chain variable region (Vbeta) on a T-cell, preferably any one of the Vbeta regions shown in Table 1.
5. A MAIT cell according to clause 4, wherein the CAR targets a plurality of T-cell receptor (TCR) beta-chain variable regions (Vbeta) on a T-cell, preferably wherein the plurality of Vbeta regions is selected from a group of Vbeta regions shown in Table 1, optionally wherein the plurality of TCR V beta regions are the same or different V beta regions.
6. A MAIT cell according to either clause 4 or clause 5, wherein the CAR targets one or more TCR Vbeta region on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20.
7. A MAIT cell according to any one of clauses 4-6, wherein the CAR is specific for a TCR Vbeta region which comprises an amino acid substantially as set out in SEQ ID No:2, or a variant or fragment thereof.
8. A MAIT cell according to any preceding clause, wherein the CAR-MAIT cell comprises one or more coding sequence, which allows for the CAR-MAIT cells to be controllably or inducibly eliminated.
9. A MAIT cell according to clause 8, wherein the one or more coding sequence encodes epidermal growth factor receptor (EGFR), or truncated epidermal growth factor receptor (tEGFR).
10. A MAIT cell according to either clause 8 or 9, wherein the one or more coding sequence encodes inducible caspase-9 (iC9).
11. A MAIT cell according to any preceding clause, wherein the MAIT cell is isolated from human peripheral blood monocyte cells (PBMCs) by magnetic activated cell sorting (MACS) and/or fluorescence activated cell sorting (FACS), more preferably both MACS and FACS.
12. A nucleic acid construct comprising a promoter operably linked to a first coding sequence, which encodes either an anti-CD4 chimeric antigen receptor (CAR) or an anti-T-cell receptor (TCR) V-beta CAR.
13. A construct according to clause 12, wherein the promoter is a PGK promoter, optionally the promoter comprises a nucleotide sequence substantially as set out in SEQ ID No: 3, or a fragment or variant thereof.
14. A construct according to either clause 12 or 13, wherein the first coding sequence encodes an anti-CD4 chimeric antigen receptor (CAR), optionally wherein the CAR is specific for a CD4 antigen which comprises an amino acid sequence substantially as set out in SEQ ID No:1, or a variant or fragment thereof.
15. A construct according to clause 14, wherein:
(i) the first coding sequence comprises a nucleotide sequence encoding an amino acid sequence substantially as set out in SEQ ID No:6, or a fragment or variant thereof;
(ii) the first coding sequence comprises a nucleotide sequence substantially as set out in SEQ ID No: 7, or a fragment or variant thereof;
(iii) the first coding sequence comprises a nucleotide sequence encoding an amino acid sequence substantially as set out in SEQ ID No:8, or a fragment or variant thereof; and/or
(iv) the first coding sequence comprises a nucleotide sequence substantially as set out in SEQ ID No: 9, or a fragment or variant thereof.
16. A construct according to either clause 12 or 13, wherein the first coding sequence encodes an anti-T-cell receptor (TCR) V-beta region CAR, optionally any of the Vbeta regions listed in Table 1.
17. A construct according to clause 16, wherein the first coding sequence encodes a plurality of T-cell receptor (TCR) beta-chain variable regions (Vbeta) CARs, preferably wherein the plurality of Vbeta regions are selected from a group of Vbeta regions shown in Table 1.
18. A construct according to either clause 16 or 17, wherein the construct comprises a coding sequence encoding at least one CAR which targets one or more TCR Vbeta region on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20, optionally wherein the construct comprises a coding sequence encoding at least one CAR which targets at least two or three TCR Vbeta regions on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20.
19. A construct according to any one of clauses 16-18, wherein the CAR is specific for a TCR Vbeta region (preferably, TCR-Vbeta 7.1 chain) which comprises an amino acid sequence substantially as set out in SEQ ID No:2, or a variant or fragment thereof.
20. A construct according to any one of clauses 16-19, wherein:
(i) the first coding sequence comprises a nucleotide sequence encoding an amino acid sequence substantially as set out in SEQ ID No: 12, or a fragment or variant thereof;
(ii) the first coding sequence comprises a nucleotide sequence substantially as set out in SEQ ID No: 13, or a fragment or variant thereof;
(iii) the first coding sequence comprises a nucleotide sequence encoding an amino acid sequence substantially as set out in SEQ ID No: 34, or a fragment or variant thereof; and/or
(iv) the first coding sequence comprises a nucleotide sequence substantially as set out in SEQ ID No: 35, or a fragment or variant thereof.
21. A construct according to any one of clauses 12-20, wherein the construct comprises a nucleotide sequence encoding a CD8a hinge and transmembrane (TM) structure domain, optionally wherein the construct comprises a nucleotide sequence encoding an amino acid sequence substantially as set out in SEQ ID No: 14, or a fragment or variant thereof and/or wherein the construct comprises a nucleotide sequence substantially as set out in SEQ ID No: 15, or a fragment or variant thereof.
22. A construct according to any one of clauses 12-21, wherein the construct comprises a nucleotide sequence encoding an intracellular domain, which comprises a signalling domain of CD28, a signalling domain of 4-1BB and/or a CD3ζ chain, and more preferably a signalling domain of CD28, a signalling domain of 4-1BB and a CD3ζ chain.
23. A construct according to clause 22, wherein:
   (i) the construct comprises a nucleotide sequence encoding an amino acid sequence substantially as set out in SEQ ID No: 16, or a fragment or variant thereof;
   (ii) the construct comprises a nucleotide sequence substantially as set out in SEQ ID No: 17, or a fragment or variant thereof;
   (iii) the construct comprises a nucleotide sequence encoding an amino acid sequence substantially as set out in SEQ ID No: 18, or a fragment or variant thereof;
   (iv) the construct comprises a nucleotide sequence substantially as set out in SEQ ID No: 19, or a fragment or variant thereof;
   (v) the construct comprises a nucleotide sequence encoding an amino acid sequence substantially as set out in SEQ ID No: 20, or a fragment or variant thereof; and/or
   (vi) the construct comprises a nucleotide sequence substantially as set out in SEQ ID No: 21, or a fragment or variant thereof.
24. A construct according to any one of clauses 12-23, wherein the nucleic acid construct comprises a second coding sequence, which encodes at least one suicide protein, and more preferably at least two suicide proteins.
25. A construct according to clause 24, wherein the second coding sequence encodes: (i) epidermal growth factor receptor (EGFR), or truncated epidermal growth factor receptor (tEGFR); and/or (ii) inducible caspase-9 (iC9).
26. A construct according to clause 25, wherein (i) the construct comprises a nucleotide sequence encoding an amino acid sequence substantially as set out in SEQ ID No: 22, or a fragment or variant thereof, optionally wherein the construct comprises a nucleotide sequence substantially as set out in SEQ ID No: 23, or a fragment or variant thereof; and/or (ii) the construct comprises a nucleotide sequence encoding an amino acid sequence substantially as set out in SEQ ID No: 24, or a fragment or variant thereof, optionally wherein the construct comprises a nucleotide sequence substantially as set out in SEQ ID No: 25, or a fragment or variant thereof.
27. A construct according to any one of clauses 12-26, wherein the construct comprises a nucleotide sequence encoding an amino acid sequence substantially as set out in SEQ ID No: 29, or a fragment or variant thereof, optionally wherein the construct comprises a nucleotide sequence substantially as set out in SEQ ID No: 30, or a fragment or variant thereof.
28. A construct according to any one of clauses 12-26, wherein the construct comprises a nucleotide sequence encoding an amino acid sequence substantially as set out in SEQ ID No: 31, or a fragment or variant thereof, optionally wherein the construct comprises a nucleotide sequence substantially as set out in SEQ ID No: 32, or a fragment or variant thereof.
29. An expression vector encoding the nucleic acid construct according to any one of clauses 12-28, optionally wherein the vector comprises a nucleic acid sequence substantially as set out in SEQ ID No: 33 or 36, or a fragment or variant thereof.
30. A method of isolating a MAIT cell, the method comprising:
   (i) providing peripheral blood monocyte cells (PBMCs); and
   (ii) subjecting the PBMCs to magnetic activated cell sorting (MACS) and/or fluorescence activated cell sorting (FACS) to isolate MAIT cells therefrom.
31. A method of producing a CAR-MAIT cell, the method comprising:
   (i) providing peripheral blood monocyte cells (PBMCs);
   (ii) subjecting the PBMCs to MACS and/or FACS to isolate MAIT cells therefrom;
   (iii) activating the isolated MAIT cells, optionally by contacting them with an anti-CD3 and/or anti-CD28 antibody; and
   (iv) transducing the activated MAIT cells with a nucleic acid encoding a CAR, to thereby produce a CAR-MAIT cell.
32. A method according to either clause 30 or 31, wherein the method comprises subjecting the PBMCs to both MACS and FACS to isolate the MAIT cells therefrom, optionally wherein the PBMCs are subjected to MACS followed by FACS.
33. A method according to any one of clauses 30-32, wherein the isolated MAIT cells are activated with an anti-CD3 antibody and an anti-CD28 antibody.
34. A method according to any one of clauses 31-33, wherein step (iv) comprises virally or retrovirally transducing the MAIT cells with a nucleic acid encoding a CAR, preferably wherein the nucleic acid encodes a CAR which targets: (i) a CD4 antigen or (ii) at least one or more TCR Vbeta region on a T-cell, preferably one or more TCR Vbeta region shown in Table 1, or one or more TCR Vbeta region on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20.
35. A method according to any one of clauses 30-43, wherein the MAIT cells are transduced with the nucleic acid construct according to any one of claims 12-28, or the expression vector according to claim 29.
36. A method according to any one of clauses 30-35, wherein the method comprises expanding the CAR-MAIT cells in a subsequent step after step (iv).
37. A CAR-MAIT cell obtained, or obtainable, by the method according to any one of clauses 30-36.
38. A pharmaceutical composition comprising a MAIT cell according to any one of clauses 1-11, or 37, and a pharmaceutically acceptable excipient.
39. The MAIT cell according to any one of clauses 1-11, or 37, or the pharmaceutical composition according to clause 38, for use in therapy.
40. The MAIT cell according to any one of clauses 1-11, or 37, or the pharmaceutical composition according to clause 38, for use in (i) immunotherapy; (ii) for treating, preventing or ameliorating cancer; (ii) for treating, preventing or ameliorating a microbial infection; or (iv) for treating, preventing or ameliorating an autoimmune disease.
41. The MAIT cell according to any one of clauses 1-11, or 37, or the pharmaceutical composition according to clause 38, for use according to either clause 39 or clause 40, for use in treating, preventing or ameliorating a T-cell malignancy, optionally a solid tumour or a liquid tumour.
42. The MAIT cell according to any one of clauses 1-11, or 37, or the pharmaceutical composition according to clause 38, for use according to clause 41, wherein the T-cell malignancy is a Peripheral T-cell lymphoma (PTCL) or a Cutaneous T-cell lymphoma (CTCL).
43. The MAIT cell according to any one of clauses 1-11, or 37, or the pharmaceutical composition according to clause 38, for use according to clause 41, wherein:
(i) the PTCL is a PTCL subtype selected from a group consisting of: Adult T-Cell Acute Lymphoblastic Lymphoma or Leukaemia (ATL); Enteropathy-Associated Lymphoma; Hepatosplenic Lymphoma; Subcutaneous Panniculitis-Like Lymphoma (SPTCL); Precursor T-Cell Acute Lymphoblastic Lymphoma or Leukaemia; and Angioimmunoblastic T-cell lymphoma (AITL);
(ii) the CTCL is a CTCL subtype selected from a group consisting of: Mycosis fungoides (MF); Sezary syndrome (SS); and CD4+small medium pleomorphic T-cell lymphoproliferative disorder.
44. The MAIT cell according to any one of clauses 1-11, or 37, or the pharmaceutical composition according to clause 38, for use according to any one of clauses 40-43, for treating, preventing or ameliorating a viral (e.g. HIV, HBV, HTLV, EBV, HPV), bacterial (e.g. TB), or fungal infection, or for treating, preventing or ameliorating an autoimmune disease, for example systemic lupus erythematosus, rheumatoid arthritis, or myasthenia gravis.
45. The MAIT cell according to any one of clauses 1-11, or 37, or the pharmaceutical composition according to clause 38, for use according to any one of clauses 40-44, wherein the use comprises triggering a sequence encoding a suicide protein, optionally wherein the method comprises administering, to the subject, an anti-EGFR antibody and/or a caspase-inducible drug (CID).
46. A process for making the pharmaceutical composition according to clause 38, the process comprising combining a therapeutically effective amount of the MAIT cell according to any one of clauses 1-1, or clause 37, and a pharmaceutically acceptable excipient.

1. A method of isolating a MAIT cell, the method comprising:
(i) providing peripheral blood monocyte cells (PBMCs); and
(ii) subjecting the PBMCs to magnetic activated cell sorting (MACS) and/or fluorescence activated cell sorting (FACS) to isolate MAIT cells therefrom.

2. A method of producing a CAR-MAIT cell, the method comprising:
(i) providing peripheral blood monocyte cells (PBMCs);
(ii) subjecting the PBMCs to MACS and/or FACS to isolate MAIT cells therefrom;
(iii) activating the isolated MAIT cells, optionally by contacting them with an anti-CD3 and/or anti-CD28 antibody; and
(iv) transducing the activated MAIT cells with a nucleic acid encoding a CAR, to thereby produce a CAR-MAIT cell.

3. A method according to any preceding claim, wherein the method comprises stimulating the PBMCs before they are subjected to MACS and/or FACS.

4. A method according to claim 3, wherein the stimulating step comprises contacting the PBMCs with (a) an antigen comprising either MR1/5-OP-RU or 5-OP-RU; and/or (b) a cytokine, preferably wherein the stimulating step comprises contacting the PBMCs with (a) an antigen comprising either MR1/5-OP-RU or 5-OP-RU; and (b) a cytokine.

5. A method according to claim 4, wherein the cytokine is an interleukin.

6. A method according to either claim 4 or 5, wherein the cytokine is one or more interleukin selected from a group consisting of IL-2, IL-7, IL-12, IL-15, IL-18 and IL-23, or any combination thereof.

7. A method according to claim 6, wherein the one or more interleukin comprises (i) IL-2; (ii) IL-12 and IL-18; (iii) IL-2, IL-12, and IL-18; (iv) IL-12, IL-18 and IL-23; (v) IL-2, IL-12, IL-18 and IL-23, or (vi) IL-7, IL-15, IL-12 and IL-18.

8. A method according to either claim 6 or 7, wherein the one or more interleukin comprises a combination of IL-12, IL-18 and IL-23.

9. A method according to any preceding claim, wherein the method comprises subjecting the PBMCs to both MACS and FACS to isolate the MAIT cells therefrom.

10. A method according to any preceding claim, wherein the PBMCs are subjected to MACS followed by FACS.

11. A method according to any preceding claim, wherein the isolated MAIT cells are activated with an anti-CD3 antibody.

12. A method according to any preceding claim, wherein the isolated MAIT cells are activated with an anti-CD28 antibody.

13. A method according to any one of claims 2-12, wherein step (iv) comprises transducing the MAIT cells with a nucleic acid encoding a chimeric antigen receptor (CAR), optionally wherein the transduction is carried out with a virus or retrovirus.

14. A method according to claim 13, wherein the nucleic acid encodes a CAR which targets a CD4 antigen on a T-cell.

15. A method according to claim 13, wherein the nucleic acid encodes a CAR which targets at least one or more TCR Vbeta region on a T-cell.

16. A method according to claim 15, wherein the one or more TCR Vbeta region is (i) shown in Table 1, and/or (ii) is selected from a group consisting of the following Vbeta regions: Vb 1, Vb 2, Vb 3, Vb 5.1, Vb 7.1, Vb 8, Vb 12, Vb 13.1, Vb 17, and Vb 20.

17. A method according to any preceding claim, wherein the method comprises expanding the CAR-MAIT cells in a subsequent step after step (iv).

18. A method according to claim 17, wherein the CAR-MAIT cell expansion step comprises harvesting the transduced CAR-MAIT cells one or two days after transduction, optionally wherein harvested cells are contacted with an interleukin, preferably IL-2, optionally wherein the interleukin is in R10 medium.

19. A CAR-MAIT cell obtained, or obtainable, by the method according to any preceding claim.

20. A mucosal-associated invariant T (MAIT) cell expressing a chimeric antigen receptor (CAR).

21. A MAIT cell according to either claim 19 or claim 20, wherein the CAR-MAIT cell expresses a CAR which targets a CD4 antigen on a T-cell, optionally wherein the CAR is specific for a CD4 antigen which comprises an amino acid substantially as set out in SEQ ID No:1, or a variant or fragment thereof.

22. A MAIT cell according to either claim 19 or claim 20, wherein the CAR-MAIT cell expresses a CAR which targets a T-cell receptor (TCR) beta-chain variable region (Vbeta) on a T-cell, optionally (i) any one of the Vbeta regions shown in Table 1, or (ii) a plurality of T-cell receptor (TCR) beta-chain variable regions (Vbeta) on a T-cell, optionally wherein the plurality of Vbeta regions is selected from a group of Vbeta regions shown in Table 1, optionally wherein the plurality of TCR V beta regions are the same or different V beta regions.

23. A MAIT cell according to claim 22, wherein the CAR targets one or more TCR Vbeta region on a T-cell selected from a group consisting of the following Vbeta regions: Vb 1, Vb 2, Vb 3, Vb 5.1, Vb 7.1, Vb 8, Vb 12, Vb 13.1, Vb 17, and Vb 20, optionally wherein the CAR is specific for a TCR Vbeta region which comprises an amino acid substantially as set out in SEQ ID No:2, or a variant or fragment thereof.

24. A MAIT cell according to any preceding claim, wherein the CAR-MAIT cell comprises one or more coding sequence, which allows for the CAR-MAIT cells to be controllably or inducibly eliminated.

25. A MAIT cell according to claim 24, wherein the one or more coding sequence encodes epidermal growth factor receptor (EGFR), or truncated epidermal growth factor receptor (tEGFR), optionally wherein the one or more coding sequence comprises a nucleotide sequence encoding an amino acid sequence substantially as set out in SEQ ID No: 22, or a fragment or variant thereof; and/or comprises a nucleotide sequence substantially as set out in SEQ ID No: 23, or a fragment or variant thereof.

26. A MAIT cell according to either claim 24 or 25, wherein the one or more coding sequence encodes inducible caspase-9 (iC9), optionally wherein the one or more coding sequence comprises a nucleotide sequence encoding an amino acid sequence substantially as set out in SEQ ID No: 24, or a fragment or variant thereof; and/or comprises a nucleotide sequence substantially as set out in SEQ ID No: 25, or a fragment or variant thereof.

27. A pharmaceutical composition comprising a MAIT cell according to any one of claims 19-26, and a pharmaceutically acceptable excipient.

28. The MAIT cell according to any one of claims 19-26, or the pharmaceutical composition according to claim 27, for use in therapy.

29. The MAIT cell according to any one of claims 19-26, or the pharmaceutical composition according to claim 27, for use in (i) immunotherapy; (ii) for treating, preventing or ameliorating cancer; (ii) for treating, preventing or ameliorating a microbial infection; or (iv) for treating, preventing or ameliorating an autoimmune disease.

30. The MAIT cell according to any one of claims 19-26, or the pharmaceutical composition according to claim 27, for use according to either claim 28 or claim 29, for use in treating, preventing or ameliorating a T-cell malignancy, optionally a solid tumour or a liquid tumour.

31. The MAIT cell according to any one of claims 19-26, or the pharmaceutical composition according to claim 27, for use according to claim 30, wherein the T-cell malignancy is a Peripheral T-cell lymphoma (PTCL) or a Cutaneous T-cell lymphoma (CTCL).

32. The MAIT cell according to any one of claims 19-26, or the pharmaceutical composition according to claim 27, for use according to claim 31, wherein:
(i) the PTCL is a PTCL subtype selected from a group consisting of: Adult T-Cell Acute Lymphoblastic Lymphoma or Leukaemia (ATL); Enteropathy-Associated Lymphoma; Hepatosplenic Lymphoma; Subcutaneous Panniculitis-Like Lymphoma (SPTCL); Precursor T-Cell Acute Lymphoblastic Lymphoma or Leukaemia; and Angioimmunoblastic T-cell lymphoma (AITL); and/or
(ii) the CTCL is a CTCL subtype selected from a group consisting of: Mycosis fungoides (MF); Sezary syndrome (SS); and CD4+small medium pleomorphic T-cell lymphoproliferative disorder.

33. The MAIT cell according to any one of claims 19-26, or the pharmaceutical composition according to claim 27, for use according to claim 29, for treating, preventing or ameliorating: (i) a viral infection, optionally HIV, HBV, HTLV, EBV, or HPV, (ii) a bacterial infection, optionally TB, or (iii) a fungal infection, or for treating, preventing or ameliorating an autoimmune disease, for example systemic lupus erythematosus, rheumatoid arthritis, or myasthenia gravis.

34. The MAIT cell according to any one of claims 19-26, or the pharmaceutical composition according to claim 27, for use according to any one of claims 28-33, wherein the use comprises triggering a sequence encoding a suicide protein, optionally wherein the method comprises administering, to the subject, an anti-EGFR antibody and/or a caspase-inducible drug (CID).

35. A process for making the pharmaceutical composition according to claim 27, the process comprising combining a therapeutically effective amount of the MAIT cell according to any one of claims 19-26, and a pharmaceutically acceptable excipient.

36. A method of stimulating MAIT cells in a culture of PBMCs, the method comprising contacting a culture of PMBCs with (a) an antigen comprising MR1/5-OP-RU or 5-OP-RU; and/or (b) one or more interleukin selected from a group consisting of IL-2, IL-7, IL-12, IL-15, IL-18 and IL-23, or any combination thereof.

37. A method according to claim 36, wherein the method comprises contacting the culture of PMBCs with (a) an antigen comprising MR1/5-OP-RU or 5-OP-RU; and (b) one or more interleukin selected from a group consisting of IL-2, IL-7, IL-12, IL-15, IL-18 and IL-23, or any combination thereof.

38. A method according to either claim 36 or 37, wherein the one or more interleukin is IL-12, IL-18 and/or IL-23.

39. A method according to claim 38, wherein the one or more interleukin is IL-12, IL-18 and IL-23.


SEQUENCE LISTING (36 sequences)

SEQ ID NO: 1 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 2 - protein, 114 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQ

SEQ ID NO: 3 - DNA, 501 bp, Artificial Sequence
  PGK promoter
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcattctccgggcctttcg

SEQ ID NO: 4 - protein, 21 aa, Artificial Sequence
  Signalling peptide
METDTLLLWVLLLWVPGSTGD

SEQ ID NO: 5 - DNA, 63 bp, Artificial Sequence
  Signalling peptide
atggagacagacacactcctgctatgggtgctgctgctctgggttccaggttccacaggtgac

SEQ ID NO: 6 - protein, 112 aa, Artificial Sequence
  First coding sequence (VL for CD4)
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIK

SEQ ID NO: 7 - DNA, 336 bp, Artificial Sequence
  First coding sequence (VL for CD4)
gacattgtgatgactcagagccccgacagcctggccgtctcactgggcgaaagggtgaccatgaattgtaaatcttctcagagcctgctgtacagtacaaaccagaaaaattacctggcctggtatcagcagaaacccggccagagccctaagctgctgatctattgggcaagtacccgagagtcaggagtgccagacagattctccgggtctggaagtggcacagacttcaccctgacaattagctccgtgcaggccgaggacgtggctgtctactattgccagcagtactatagctaccgaactttcggcgggggaaccaaactggaaatcaag

SEQ ID NO: 8 - protein, 122 aa, Artificial Sequence
  First coding sequence (VH for CD4)
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSS

SEQ ID NO: 9 - DNA, 366 bp, Artificial Sequence
  First coding sequence (VH for CD4)
caggtgcagctgcagcagtccggaccagaggtggtcaaacccggcgctagcgtcaaaatgtcctgtaaggcatctggctacactttcacctcttatgtgattcactgggtcagacagaagcctgggcagggactggactggatcgggtacattaacccatataatgatggaactgactacgatgaaaagtttaaaggcaaggccacactgacttccgacacctcaacaagcactgcttatatggagctgtctagtctgaggtctgaagacacagcagtgtactattgcgcccgcgagaaggataactacgccactggcgcttggtttgcatattggggccaggggaccctggtgacagtctcatcc

SEQ ID NO: 10 - protein, 15 aa, Artificial Sequence
  G4S linker sequence
GGGGSGGGGSGGGGS

SEQ ID NO: 11 - DNA, 44 bp, Artificial Sequence
  G4S linker sequence
gaggaggaggcagtggcggaggagggtcaggaggaggaggaagc

SEQ ID NO: 12 - protein, 119 aa, Artificial Sequence
  First coding sequence (VL for TCR V-beta)
QVQLQQPGAELVKPGASVKMSCKASGYTFTRYWITWVKQRPGQGLEWIGDIYPGSGFTKYNEKFKSKATLTVDTSSSTAYMQLSSLTSEDSAVYYCAREGGNYWYFDVWGTGTTVTVSS

SEQ ID NO: 13 - DNA, 357 bp, Artificial Sequence
  First coding sequence (VL for TCR V-beta)
caagttcagctgcaacagcctggcgccgagcttgtgaaacctggcgcctctgtgaagatgagctgcaaggcctccggctacaccttcaccagatactggatcacctgggtcaagcagaggcctggacagggactcgagtggatcggcgatatctatcctggctccggcttcaccaagtacaacgagaagttcaagagcaaggccacactgaccgtggacaccagcagcagcacagcctacatgcagctgtctagcctgaccagcgaggacagcgccgtgtactactgtgctagagaaggcggcaactactggtacttcgacgtgtggggcaccggcaccacagtgacagttagttct

SEQ ID NO: 14 - protein, 83 aa, Artificial Sequence
  CD8a hinge and transmembrane domain
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 15 - DNA, 249 bp, Artificial Sequence
  CD8a hinge and transmembrane domain
ttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaac

SEQ ID NO: 16 - protein, 41 aa, Artificial Sequence
  CD28 signalling domain
RSKRSRLLHSDYMNMTPRRPGPTRKHYQPYAPPRDFAAYRS

SEQ ID NO: 17 - DNA, 123 bp, Artificial Sequence
  CD28 signalling domain
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 18 - protein, 47 aa, Artificial Sequence
  4-1BB signalling domain
RFSVVKRGRKKLLYIFKQPFMRPVQTTQEEDGCSCRFPEEEEGGCEL

SEQ ID NO: 19 - DNA, 141 bp, Artificial Sequence
  4-1BB signalling domain
cgtttctctgttgttaaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 20 - protein, 112 aa, Artificial Sequence
  CD3zeta chain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 336 bp, Artificial Sequence
  CD3zeta chain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 357 aa, Artificial Sequence
  Truncated EGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 23 - DNA, 1071 bp, Artificial Sequence
  Truncated EGFR
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggaacccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 24 - protein, 413 aa, Artificial Sequence
  Inducible caspase-9 (iC9)
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDYPYDVPDYALD

SEQ ID NO: 25 - DNA, 1242 bp, Artificial Sequence
  Inducible caspase-9 (iC9)
atgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaa

SEQ ID NO: 26 - protein, 22 aa, Artificial Sequence
  P2A spacer sequence
GSGATNFSLLKQAGDVEENPGP

SEQ ID NO: 27 - DNA, 66 bp, Artificial Sequence
  P2A spacer sequence
ggatccggagccacgaacttctctctgttaaagcaagcaggagacgtggaagaaaaccccggtcct

SEQ ID NO: 28 - DNA, 592 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 29 - protein, 1370 aa, Artificial Sequence
  Nucleic acid construct (CART4)
METDTLLLWVLLLWVPGSTGDDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKGGGGSGGGGSGGGGSQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGATNFSLLKQAGDVEENPGPMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDYPYDVPDYALD

SEQ ID NO: 30 - DNA, 4113 bp, Artificial Sequence
  Nucleic acid construct (CART4)
atggagacagacacactcctgctatgggtgctgctgctctgggttccaggttccacaggtgacgacattgtgatgactcagagccccgacagcctggccgtctcactgggcgaaagggtgaccatgaattgtaaatcttctcagagcctgctgtacagtacaaaccagaaaaattacctggcctggtatcagcagaaacccggccagagccctaagctgctgatctattgggcaagtacccgagagtcaggagtgccagacagattctccgggtctggaagtggcacagacttcaccctgacaattagctccgtgcaggccgaggacgtggctgtctactattgccagcagtactatagctaccgaactttcggcgggggaaccaaactggaaatcaagggaggaggaggcagtggcggaggagggtcaggaggaggaggaagccaggtgcagctgcagcagtccggaccagaggtggtcaaacccggcgctagcgtcaaaatgtcctgtaaggcatctggctacactttcacctcttatgtgattcactgggtcagacagaagcctgggcagggactggactggatcgggtacattaacccatataatgatggaactgactacgatgaaaagtttaaaggcaaggccacactgacttccgacacctcaacaagcactgcttatatggagctgtctagtctgaggtctgaagacacagcagtgtactattgcgcccgcgagaaggataactacgccactggcgcttggtttgcatattggggccaggggaccctggtgacagtctcatccgcggccgcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatccggagccacgaacttctctctgttaaagcaagcaggagacgtggaagaaaaccccggtcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggaacccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggatctggagccacgaacttctctctgttaaagcaagcaggagacgtggaagaaaaccccggtcctatgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaa

SEQ ID NO: 31 - protein, 1366 aa, Artificial Sequence
  Nucleic acid construct (CARTVb7.1)
MALPVTALLLPLALLLHAARPDIQMTQSPSSLSASLGGKVTLTCKASQDINKYIAWYQHKPGKGPRLLIHYTSTLQPGIPSRFSGSGSGRDYSFSISNLEPEDVATYYCLQYDNLRTFGGGTKLEIKRTDGGGGSGGGGSGGGGSQVQLQQPGAELVKPGASVKMSCKASGYTFTRYWITWVKQRPGQGLEWIGDIYPGSGFTKYNEKFKSKATLTVDTSSSTAYMQLSSLTSEDSAVYYCAREGGNYWYFDVWGTGTTVTVSSAAAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGATNFSLLKQAGDVEENPGPMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDYPYDVPDYALD

SEQ ID NO: 32 - DNA, 4101 bp, Artificial Sequence
  Nucleic acid construct (CARTVb7.1)
atggctctgcctgttacagctctgctgctgcctctggctctgcttctgcatgccgccagacctgacatccagatgacacagagccctagcagcctgtctgcctctctcggcggaaaagtgaccctgacatgcaaggccagccaggacatcaacaagtatatcgcctggtatcagcacaagcccggcaagggacctagactgctgatccactacaccagcacactgcagcctggcatccccagcagattttctggcagcggctccggcagagactacagcttcagcatcagcaacctggaacctgaggacgtggccacctactactgcctgcagtacgacaacctgcggacctttggcggcggaacaaagctggaaatcaagcggacagatggcggaggcggatcaggcggcggaggaagcggtggcggaggatctcaagttcagctgcaacagcctggcgccgagcttgtgaaacctggcgcctctgtgaagatgagctgcaaggcctccggctacaccttcaccagatactggatcacctgggtcaagcagaggcctggacagggactcgagtggatcggcgatatctatcctggctccggcttcaccaagtacaacgagaagttcaagagcaaggccacactgaccgtggacaccagcagcagcacagcctacatgcagctgtctagcctgaccagcgaggacagcgccgtgtactactgtgctagagaaggcggcaactactggtacttcgacgtgtggggcaccggcaccacagtgacagttagttctgcggccgcggccgcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatccggagccacgaacttctctctgttaaagcaagcaggagacgtggaagaaaaccccggtcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggaacccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggatctggagccacgaacttctctctgttaaagcaagcaggagacgtggaagaaaaccccggtcctatgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaa

SEQ ID NO: 33 - DNA, 10348 bp, Artificial Sequence
  Vector (CART4 CAR4-tEGFR-iC9)
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctctcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcattctccgggcctttcgacctgcagcccaagccaccatggagacagacacactcctgctatgggtgctgctgctctgggttccaggttccacaggtgacgacattgtgatgactcagagccccgacagcctggccgtctcactgggcgaaagggtgaccatgaattgtaaatcttctcagagcctgctgtacagtacaaaccagaaaaattacctggcctggtatcagcagaaacccggccagagccctaagctgctgatctattgggcaagtacccgagagtcaggagtgccagacagattctccgggtctggaagtggcacagacttcaccctgacaattagctccgtgcaggccgaggacgtggctgtctactattgccagcagtactatagctaccgaactttcggcgggggaaccaaactggaaatcaagggaggaggaggcagtggcggaggagggtcaggaggaggaggaagccaggtgcagctgcagcagtccggaccagaggtggtcaaacccggcgctagcgtcaaaatgtcctgtaaggcatctggctacactttcacctcttatgtgattcactgggtcagacagaagcctgggcagggactggactggatcgggtacattaacccatataatgatggaactgactacgatgaaaagtttaaaggcaaggccacactgacttccgacacctcaacaagcactgcttatatggagctgtctagtctgaggtctgaagacacagcagtgtactattgcgcccgcgagaaggataactacgccactggcgcttggtttgcatattggggccaggggaccctggtgacagtctcatccgcggccgcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatccggagccacgaacttctctctgttaaagcaagcaggagacgtggaagaaaaccccggtcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggaacccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggatctggagccacgaacttctctctgttaaagcaagcaggagacgtggaagaaaaccccggtcctatgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 34 - protein, 109 aa, Artificial Sequence
  First coding sequence (VH for TCR V-beta)
DIQMTQSPSSLSASLGGKVTLTCKASQDINKYIAWYQHKPGKGPRLLIHYTSTLQPGIPSRFSGSGSGRDYSFSISNLEPEDVATYYCLQYDNLRTFGGGTKLEIKRTD

SEQ ID NO: 35 - DNA, 327 bp, Artificial Sequence
  First coding sequence (VH for TCR V-beta)
gacatccagatgacacagagccctagcagcctgtctgcctctctcggcggaaaagtgaccctgacatgcaaggccagccaggacatcaacaagtatatcgcctggtatcagcacaagcccggcaagggacctagactgctgatccactacaccagcacactgcagcctggcatccccagcagattttctggcagcggctccggcagagactacagcttcagcatcagcaacctggaacctgaggacgtggccacctactactgcctgcagtacgacaacctgcggacctttggcggcggaacaaagctggaaatcaagcggacagat

SEQ ID NO: 36 - DNA, 10336 bp, Artificial Sequence
  Vector (CARTVb7.1 CARTVb7.1-tEGFR-iC9)
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctctcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcattctccgggcctttcgacctgcagcccaagccaccatggctctgcctgttacagctctgctgctgcctctggctctgcttctgcatgccgccagacctgacatccagatgacacagagccctagcagcctgtctgcctctctcggcggaaaagtgaccctgacatgcaaggccagccaggacatcaacaagtatatcgcctggtatcagcacaagcccggcaagggacctagactgctgatccactacaccagcacactgcagcctggcatccccagcagattttctggcagcggctccggcagagactacagcttcagcatcagcaacctggaacctgaggacgtggccacctactactgcctgcagtacgacaacctgcggacctttggcggcggaacaaagctggaaatcaagcggacagatggcggaggcggatcaggcggcggaggaagcggtggcggaggatctcaagttcagctgcaacagcctggcgccgagcttgtgaaacctggcgcctctgtgaagatgagctgcaaggcctccggctacaccttcaccagatactggatcacctgggtcaagcagaggcctggacagggactcgagtggatcggcgatatctatcctggctccggcttcaccaagtacaacgagaagttcaagagcaaggccacactgaccgtggacaccagcagcagcacagcctacatgcagctgtctagcctgaccagcgaggacagcgccgtgtactactgtgctagagaaggcggcaactactggtacttcgacgtgtggggcaccggcaccacagtgacagttagttctgcggccgcggccgcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatccggagccacgaacttctctctgttaaagcaagcaggagacgtggaagaaaaccccggtcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggaacccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggatctggagccacgaacttctctctgttaaagcaagcaggagacgtggaagaaaaccccggtcctatgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa